(19) United States
(12) Patent Application Publication
(10) Pub. No.: US 2013/0102943 A1
(43) Pub. Date: Apr. 25, 2013

(54) RELOADABLE FINISHES FOR TEXTILES AND FORMULATIONS FOR LOADING SUCH FINISHES

(71) Applicants: Theo Gaupp, Untervaz (CH); Roland Lottenbach, Staad (CH); Hans-Jürgen Hübner, Brione s.M. (CH); Sabine Dabzer, Sevelen (CH)

(72) Inventors: Uwe Holzdörfer, Sevelen (CH); Theo Gaupp, Untervaz (CH); Roland Lottenbach, Staad (CH); Hans-Jürgen Hübner, Brione s.M. (CH)

(73) Assignee: SCHOELLER TEXTIL AG, Sevelen (CH)

(21) Appl. No.: 13/394,539

(22) PCT Filed: Aug. 25, 2010
(86) PCT No.: PCT/EP2010/062381

§ 371 Date: Jan. 3, 2013

(30) Foreign Application Priority Data
Sep. 8, 2009 (CH) 01391/09

(51) Int. Cl. A61K 9/107; D06M 15/29; A61F 13/00

(52) U.S. Cl. CPC A61K 9/107; A61F 13/00; D06M 15/29
USPC 602/42; 442/59; 523/105; 514/785; 424/400; 424/450; 427/2.31

(57) ABSTRACT

A polymer compound according to the invention comprises an acrylic acid copolymer composed of acrylic acid derivatives and/or methacrylic acid derivatives, containing: a) at least one acrylic acid derivative and/or methacrylic acid derivative substituted with a sulfonic acid group; b) at least one hydrophilically substituted acrylic acid derivative and/or methacrylic acid derivative; c) at least one lipophilically substituted acrylic acid derivative and/or methacrylic acid derivative; and d) at least one acrylic acid derivative and/or methacrylic acid derivative which acts as a crosslinking agent. In the method according to the invention for loading textile products with a low-molecular compound, a) a textile product is provided with a finishing layer whose accessible surface has a negative charge; and b) the textile product is brought together with an emulsion or active substance solution, for example by immersing the textile product in the emulsion/solution, or by spraying the emulsion/solution on the textile product. At least one low-molecular compound is contained in the dispersed phase of the emulsion, and the surface of the particles of the dispersed phase has a positive charge.

### TECHNICAL FIELD

The invention relates to polymer compounds and finishing formulations for finishing textile products, the corresponding finishing layers and textile products, emulsions for loading the finishing layer with active substances, and a method for loading finished textiles with low-molecular compounds.

### PRIOR ART

Numerous finishes of textile products for providing textiles with additional functional properties are known from the prior art. The terms “textiles” and “textile products” are understood in particular to mean fibers as well as manufactured textile products (for example, woven fabrics, knitted fabrics, nonwoven fabrics, etc.) which may be present, for example, as cloth or an already processed product (articles of clothing, for example). The textiles may be made of any given, known materials, in particular natural and/or synthetic materials, in particular cotton, linen, silk, hemp, jute, wool, sisal, viscose, polyamide, polyester, etc., and mixtures thereof.

Within the scope of the present invention, the term “textile products” is expressly intended to also include wound dressings, for example adhesive dressings (bandages), as well as bandage materials.

It is known that textiles may be loaded with substances and active substances. For example, textiles may be finished with cyclodextrins as active substance carrier, and low-molecular active substances may be incorporated into these cyclodextrins, from which they are subsequently released. Thus, active substances are able to migrate from a textile product onto the skin of the wearer, where they may have a certain desired effect. For example, cosmetic and/or medical active substances may be absorbed transdermally in this manner.

Likewise, textiles may be loaded with antibacterial or fungicidal substances, for example to prevent odor formation, or with UV-absorbent substances in order to increase the UV absorption of the fabric. Substances which repel insects are also conceivable.

For textiles loaded with low-molecular active substances, a portion of the active substances are unavoidably lost during each laundering operation. For such functionally modified textiles, it is therefore desirable to be able to reload the textiles with active substances in a simple manner.

Also known is the finishing of textiles with so-called microcapsules which contain low-molecular active substances, as well as the individual loading of textiles with such microcapsules. Microcapsules have the disadvantage that the active substances are abruptly released as the result of mechanical action and destruction of the microcapsules. Therefore, such finishes are not very suitable for controlled delivery over an extended period of time.

### OBJECT OF THE INVENTION

The object of the invention is to provide an advantageous finishing formulation which does not have the above-mentioned and other disadvantages of the prior art.

It is an object of the invention in particular to provide a finishing formulation by means of which textile products and/or wound dressings may be provided with a finish that is loadable with cationic microemulsions and/or cationic active substances in solution in an efficient and targeted manner. The loading may preferably be carried out multiple times. The droplets of the microemulsion may in particular contain active substances and other active ingredients.

It is the aim that the active substances of the finishing layer are released at a defined desorption rate.

A further object of the invention is to provide polymer compounds for such a finishing formulation, and to provide such a finish.

A further object of the invention is to provide a microemulsion by means of which a finishing layer according to the invention may be loaded with low-molecular compounds, in particular active substances. Such loading may preferably take place with a high dilution of the emulsion, for example during a laundering operation.

These and other objects are achieved by a polymer compound according to the invention, a finishing formulation containing such a polymer compound, a finishing layer containing such a polymer compound, a finished textile product or a coated wound dressing, an emulsion according to the invention for loading the finishing layer with active substances, and a method according to the invention according to the independent claims. Further preferred embodiments and variants are stated in the dependent claims.

### DESCRIPTION OF THE INVENTION

A polymer compound according to the invention comprises an acrylic acid copolymer composed of acrylic acid derivatives and/or methacrylic acid derivatives, containing: a) at least one acrylic acid derivative and/or methacrylic acid derivative substituted with a sulfonic acid group; b) at least one hydrophilically substituted acrylic acid derivative and/or methacrylic acid derivative; c) at least one lipophilically substituted acrylic acid derivative and/or methacrylic acid derivative; and d) at least one acrylic acid derivative and/or methacrylic acid derivative which acts as a crosslinking agent.

One possible example of an acrylic acid derivative monomer or methacrylic acid derivative monomer containing a sulfonic acid group is 2-acryloyl-2-methylpropanesulfonic acid. The sulfonic acid groups of the polymer compound according to the invention provide negative charge sites in the polymer matrix, similarly as for an ion exchange polymer. At the pH values that are customary during laundering, the charge sites are deprotonated due to the very low pK value of the sulfonic acid groups. The negative charge sites in the polymer matrix result in a negative surface charge, which is naturally balanced by cations. Without being limited to a specific explanation, the effect according to the invention of the polymer compound is that dispersed particles, charged with a positive surface charge, of a microemulsion or also positively charged active substances, for example hydrochlorides of heterocyclic compounds or other cationic compounds, may be incorporated into the matrix of the polymer compound according to the invention. On account of the negative charge sites, the polymer matrix has a negative surface charge. In this manner, the dispersed phase of a corresponding emulsion or the cationic compounds may be efficiently incorporated into a finishing layer according to the invention.

The dispersed phase of such an emulsion contains low-molecular compounds which are intended to have a certain effect. The mentioned compounds may be re-emitted from the finishing layer in a controlled manner, i.e., continuously over an extended period of time, or may remain in place. For example, after a textile is loaded, active substances may pass from the textile onto the skin of the wearer, where they are transdermally absorbed and develop their specific effect. The desorption behavior may be customized by controlling the hydrophilicity/lipophilicity ratio, i.e., by adjusting the amphiphilic properties of the donor layer. Thus, for example, it is possibly to easily adjust the desorption time to 16 h, which corresponds to a realistic time for wearing textiles on the body, or to selectively adjust the desorption time to any given desired period of time.

When the textile loaded with the active substance is worn directly on the body, the unloading process is triggered by heat, friction, and moisture. A finishing layer according to the invention, also referred to as a donor layer, also has the property that the desorption may be triggered by the salts in body perspiration. In particular, the sodium ions present in perspiration increase the active substance delivery from the donor layer to the skin. Locations on the body which are accordingly stressed, for example during athletic activity, may thus preferably be supplied with active substances.

A finishing layer according to the invention may be unloaded and reloaded as often as desired.

The loading of a textile product finished with a polymer compound according to the invention may also take place at a comparatively high dilution of the emulsion. Thus, for example, such an emulsion may be added, similarly to a fabric softener, to the rinse water in the last rinse cycle of a laundering program of a household washing machine. However, loading may be also be carried out by hand washing or by spraying the textiles, which may be advantageous, depending on the application. For wound dressings, the coating is preferably applied in the sterile environment of the manufacturing facility. For bandage materials, reloading similarly as for clothing is possible.

Analogously, it would be conceivable for the polymer matrix to have positive charge sites, for example in the form of quaternary ammonium groups, instead of the sulfonic acid groups, so that emulsion particles having a negative surface charge are adsorbed thereon. However, such a variant has the disadvantage that during laundering of an accordingly finished textile product, the customary anionic surfactants are able to accumulate at the positive charge sites, as the result of which these charge sites are shielded and no longer accessible.

To optimize the loading capacity of a finishing layer, the largest possible number of charge sites should be accessible to the particles of the emulsion. For this purpose, it is therefore advantageous when the finishing layer on the textile has a certain swelling, since the accessible surface of the polymer matrix, and therefore also the available surface charge, is thus increased.

To achieve the desired hydration and swelling capabilities of the finishing layer according to the invention, the polymer compound according to the invention contains hydrophilically substituted acrylic acid derivative monomers or methacrylic acid derivative monomers, for example ethyl triglycol methacrylate, 2-hydroxyethyl methacrylate (HEMA), and/or mPEG methacrylate, in particular mPEG 1000 methacrylate and mPEG 350 methacrylate. HEMA has the additional effect that it acts as a docking site for crosslinking monomers when the finishing layer is fixed on the textile, resulting in internal polymer crosslinking. As a whole, finishing layers containing HEMA are rather brittle when they are not hydrated, while finishing layers containing mPEG methacrylates remain fairly elastic in the dry state.

Lipophilically substituted acrylic acid derivative monomers or methacrylic acid derivative monomers, for example 2-ethylhexyl acrylate, ensure a certain lipophilicity of the matrix of a finishing layer according to the invention. The ratio of the fractions of hydrophilic and lipophilic monomers determines, among other things, the absorption and desorption properties of a finishing layer according to the invention. In addition, lipophilic compounds within the adsorbed emulsion particles are able to migrate into the lipophilic domains of the polymer matrix, thus increasing the loading capacity of the finishing layer.

To achieve sufficient permanence of the finishing layer on the textile product, the finishing layer must be fixed on the fibers. For this purpose, the polymer compound according to the invention is crosslinked with the textile fibers. Crosslinking agents for textile finishes are known from the prior art. One possible example of a crosslinking monomer for a polymer compound according to the invention is N-(butoxymethyl)acrylamide. For thermal and/or acid-catalyzed fixing, the corresponding monomer is covalently bonded to OH and NH₂ groups in the fibers.

The crosslinking monomers of the acrylic acid copolymer are advantageously selected from a group composed of N-(butoxymethyl)acrylamide, N-(methylol)acrylamide, glycidyl methacrylate, p-EMKO-TDI-o-HEMA, and EMKO-2-(N-(tert-butyl){[(3-isocyanato-1,5,5-trimethylcyclohexyl)methyl]amino}carbonylamino)ethyl methacrylate.

A polymer compound according to the invention may contain further polymer compounds, for example polyethersulfones, polyurethanes, polyester urethanes, polyether urethanes, polyamides, or mixtures thereof. A polymer compound according to the invention may be a blend of such various polymer compounds, which are advantageously crosslinkable.

To produce a finishing layer according to the invention on a textile product or on a wound dressing, a finishing formulation is applied to the textile product or the wound dressing, for example in an aqueous bath. The finishing formulation contains a polymer compound according to the invention in dissolved form and/or in the form of a microemulsion. After a first drying step, the polymer compound is thermally and/or acid catalytically fixed on the fiber substrate. The appropriate methods from the prior art for finishing textiles with polymer coatings are known to those skilled in the art.

In addition to polymer compounds according to the invention, a finishing formulation according to the invention may contain further polymers such as PES, PU, PUE, PA, crosslinking agent systems, etc. and/or mixtures thereof. Due to their amphiphilic structure, the polymer compounds according to the invention are mixable and/or crosslinkable with numerous polymers, or processable to form blends.

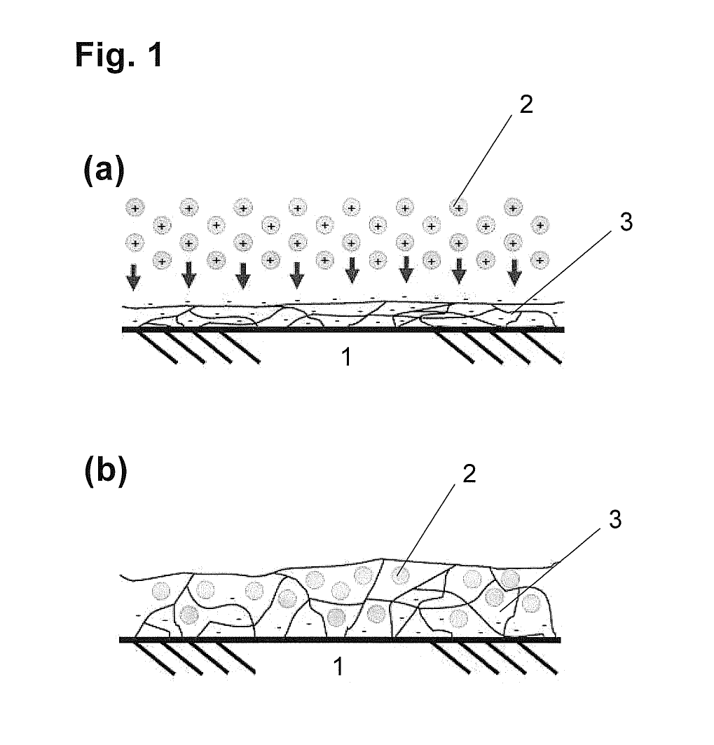

The adsorption of emulsion particles in a finishing layer according to the invention is schematically illustrated in FIG. 1. The finishing layer 3, which is fixed on the fiber substrate, 1 is hydrated and swelled by the absorption of water from the environment, for example atmospheric humidity or rinse water from the loading process. The negative charge sites in the polymer matrix are accessible. When the finishing layer 3 is now brought together with an emulsion, the emulsion particles 2 of which have a positive surface charge, these particles 2 are able to migrate into the pores and gaps in the polymer matrix (FIG. 1(a)), where they are incorporated into the polymer matrix (FIG. 1(b)).

In a method according to the invention for loading textile products with low-molecular compounds, a) a textile product is provided with a finishing layer whose accessible surface has a negative charge; b) the textile product is brought together with an emulsion, for example by immersing the textile product in the emulsion or by spraying the emulsion on the textile product. At least one low-molecular compound is contained in the dispersed phase of the emulsion. The boundary surface of the dispersed phase has a positive charge. Instead of an emulsion, a solution may, be used when the low-molecular compound dissolved therein is cationic.

Step b) is preferably carried out multiple times at any desired intervals. In this way, the finished materials may be repeatedly reloaded with the desired low-molecular compounds.

The emulsion containing the low-molecular compound is preferably an emulsion according to the invention, as described below.

An emulsion according to the invention contains at least one low-molecular compound with which the textiles are to be loaded in the dispersed phase of the emulsion. The surface of the particles of the dispersed phase has a positive charge. This positive charge is naturally balanced by negatively charged counterions. The surface charge of the particles of the dispersed phase of the emulsion is advantageously at least 15 mC/g emulsion, particularly advantageously at least 90 mC/g emulsion.

The positive surface charge of the emulsion particles is achieved using emulsifiers or surface-active compounds having a positive charge at their polar end. For an oil-in-water emulsion, these positive charges are located at the surface of the particles. Examples of suitable surface-active compounds are lecithin, in particular phosphatidylcholine lecithins, and/or quaternary ammonium compounds containing one or two long-chain lipophilic radicals, in particular behenyl trimethylammonium or ethyl-N-alpha-lauroyl-L-arginate HCl.

In order for the particles of the dispersed phase to be able to penetrate into the swelled polymer matrix of the finishing layer, the diameter of the particles should not exceed a certain size. The smaller the particles, the better and more rapidly they are able to penetrate into the pores of the polymer matrix and accumulate on the matrix. At least 90 vol-% of the particles of the dispersed phase of the emulsion preferably have a hydrodynamic diameter of less than 1000 nm, particularly preferably less than 700 nm.

The emulsion may be an oil-in-water emulsion, at least one low-molecular compound being present in the lipophilic dispersed phase. This variant is particularly suitable for lipophilic low-molecular compounds.

Water-in-oil-in-water emulsions, for example, are suitable for hydrophilic low-molecular compounds, at least one low-molecular compound in the aqueous dispersed phase being present within the lipophilic dispersed phase. Emulsions containing liposomes are also suitable, in this case the hydrophilic low-molecular compounds in the aqueous phase being present within the liposomes.

If lipophilic as well as hydrophilic compounds are to be loaded on the textile, the various types of emulsions may also be combined. Alternatively, different emulsions may be used in sequence.

The invention further relates to the use of an emulsion according to the invention for loading a textile product or a wound dressing with lipophilic and/or hydrophilic low-molecular compounds, the textile product or the wound dressing preferably having a finishing layer according to the invention, and/or having been finished with a finishing formulation according to the invention.

A finishing layer according to the invention may also be combined with the 3×DRY® technology of the present applicant disclosed in WO 2002/075038 A1. Thus, for example, the hydrophilic finishing layer according to the invention may be provided on the outer side with a hydrophobic coating. In this manner, a textile according to the invention not only may be made water-repellent on an outer surface while it is still hydrophilic on the interior, but at the same time the hydrophobic layer may also be used as an active substance barrier with respect to the outside. Such barrier layers are common for transdermal bandages, for example, in which active substances are intended to be desorbed only in a defined direction.

A finishing layer according to the invention also has the property of being able to bind cationic heavy metal ions such as cadmium or lead, or other toxic substances. This is particularly important, for example, in countries having high arsenic levels in drinking water, since in this manner drinking water may be made safe to drink. The textile finished with the polymer layer may be regenerated using salt, sea water, soap, or laundry detergent. In addition, a finishing layer according to the invention may bind organic impurities in water, for example diesel fuel or gasoline, since organic impurities are able to adsorb to the lipophilic structures of the amphiphilic polymer compounds according to the invention. As a result of this application, a textile according to the invention may be used for treating drinking water.

### Carrying Out the Invention

The following examples are used for explanation, but should not be construed as limiting the invention to the features disclosed herein.

### A. Polymers

### Polymer Compound P-002

Polymer compound P-002 is an acrylic acid copolymer, namely, poly(acrylic acid-stat-2-ethylhexyl acrylate-stat-N-(butoxymethyl)acrylamide). This compound contains the monomers acrylic acid, whose carboxylic acid group is used to provide negative charge sites in the polymer matrix. 2-ethylhexyl acrylate as a lipophilic group, and N-(butoxymethyl)acrylamide. The latter is used for crosslinking the polymer with OH and NH₂ groups in the textile fibers. The synthesis is carried out by radical emulsion polymerization.

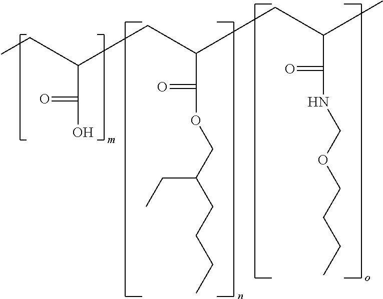

### Starting Product Solution:

| Weigh-in | | Product description |
|---|---|---|
| 30.0 | g | 99.5% acrylic acid, stabilized, Acros Organics, M = 72.06 |
| | | g/mol, d = 1.050 g/mL, BP = 139° C. |
| 115.5 | g | 99% 2-ethylhexyl acrylate, stabilized, Acros Organics, |
| | | d = 0.880 g/mol, BP = 215° C. |
| 4.5 | g | Cylink NBMA monomer: 81% N-(butoxymethyl)acrylamide, |
| | | Cytec Industries, d = 0.96 g/mL, BP = 125-128° C./0.03 |
| | | mm Hg |
| 4.5 | g | Disponil AFX 1080, Cognis Deutschland GmbH & Co. KG |
| 0.75 | g | Sodium dodecyl sulfate (SDS), p.a., Serva Feinbiochemika |
| 25.0 | g | Dipropylene glycol, d = 1.023 g/mL, BP = 90-95° C./ |
| | | 1 mm Hg |
| 275.0 | g | Deionized water |

Initiator solution (corresponds to 1 wt.-% V-501 relative to monomers and crosslinking agent):

| Weigh-in | Product description |
|---|---|
| 1.5 g | V-501: ≧98.0% 4,4′-azobis-(4-cyanovaleric acid), |
| | Fluka, M = 280.28 g/mol |
| 1.0 g | 50% sodium hydroxide |
| 50.0 g | Deionized water |

Apparatus:

1-L four-neck round bottom flask with stirrer, reflux condenser, septum, and temperature sensor. A discharge line with a stopcock on the reflux condenser was used for evacuation and flushing with nitrogen.

Prehomogenization:

The starting product solution was passed through a high-pressure homogenizer five times at 600 bar, resulting in an emulsion having a bluish sheen. 1 g homogenized starting product solution was diluted 1:40 with 39 g water. Viscosity: 1.01 mPa·s. The particle size distribution was determined by photon correlation spectroscopy (PCS) (Malvern Instruments Ltd., Malvern, Worcestershire, WR14 IXZ, UK; model: Zetasizer Nano-S ZEN 1600): peak 1: d(H)=1140 nm−98.1 vol-%; peak 2: d(H)=5290 nm−1.9 vol-%.

### Synthesis:

455 g homogenized starting product solution was placed in the apparatus and heated to 90° C. The apparatus was then evacuated and flushed with nitrogen three times each to remove atmospheric oxygen (inhibitor). The apparatus was back-flushed with nitrogen to ensure pressure compensation over the entire polymerization. 25 g initiator solution was then metered through the septum via a syringe, with good stirring. The reaction proceeded immediately. Due to the strongly exothermic reaction (5° C./min), cooling was provided immediately via water bath, and the reaction temperature was held constant at 90° C. (no subsequent exothermic conditions were detectable). After the initial exothermic conditions subsided, after 40 min the remaining 27 g initiator solution was added via syringe (no subsequent exothermic conditions were detectable).

### In-Process Control:

After a total reaction time of 4 h, 2 g reaction solution was withdrawn from the apparatus, placed on a tared aluminum dish, and dried in a drying oven at 180° C. for 30 min: Weigh-in =2.023 g of a low-viscosity white emulsion solution; weigh-out=0.594 g of a milky white polymer; Solids content: 29.36% (TS_{100% theoretical conversion}: 30.87%)=95.1% conversion. Swelling behavior: Slightly dried polymer was combined with THF in a test tube; slight swelling was visually detectable. pH=3.3.

Reaction Solution:

The low-viscosity reaction solution having a bluish-pink sheen was cooled to room temperature. 1 g reaction solution was diluted 1:40 with 39 g water. Viscosity: 1.05 mPa·s. Particle size distribution (PCS measurement): peak 1: d(H)=503 nm−92.6 vol-%; peak 2: d(H)=5220 nm−7.4 vol-%. 1 mL 25% ammonia solution was added to the 1:40 diluted reaction solution to determine the accessibility of the carboxyl groups, i.e., the swelling capability of the polymer. Viscosity: 2.36 mPa·s. Particle size distribution (PCS measurement): peak 1: d(H)=587 nm−93.3 vol-%; peak 2: d(H)=5340 nm−6.7 vol-%. Emulsion droplet volume (calculated): peak 1: V(H)=66.6E+6 nm³ without NH₃; peak 1: V(H)=105.9E+6 nm³ with NH₃; swelling emulsion droplets: peak 1: 59% increase in volume.

### Polymer Compound P-004

Polymer compound P-004, poly(2-acryloylamino-2-methylpropane sodium sulfonate-stat-2-ethylhexyl acrylate-stat-N-(butoxymethyl)acrylamide), contains the monomers 2-acryloylamino-2-methylpropane sodium sulfonate, 2-ethylhexyl acrylate, and N-(butoxymethyl)acrylamide. The sulfonate groups provide the negative charge sites in the polymer matrix.

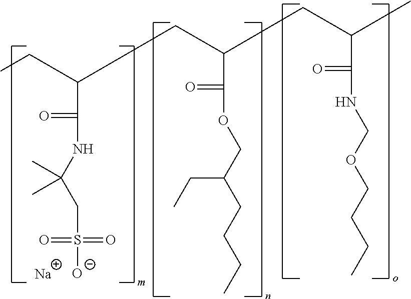

### Starting Product Solution:

| Weigh-in | | Product description |
|---|---|---|
| 30.0 | g | >98.0% 2-acryloylamino-2-methylpropanesulfonic acid, |
| | | Fluka |
| 115.5 | g | 99% 2-ethylhexyl acrylate, stabilized, Acros Organics, |
| | | d = 0.880 g/mL, BP = 215° C. |
| 4.5 | g | Cylink NBMA monomer: 81% N-(butoxymethyl)acrylamide, |
| | | Cytec Industries |
| 4.5 | g | Disponil AFX 1080, Cognis Deutschland GmbH & Co. KG |
| 0.75 | g | Sodium dodecyl sulfate (SDS), p.a., Serva Feinbiochemika |
| 25.0 | g | Dipropylene glycol, d = 1.023 g/mL, BP = 90-95° C./ |
| | | 1 mm Hg |
| 12.7 | g | 50% sodium hydroxide (water content taken into account) |
| 262.3 | g | Deionized water |

Prehomogenization:

The starting product solution (pH=6.5) was passed through the high-pressure homogenizer five times at 600 bar, resulting in an emulsion having a bluish sheen. 1 g homogenized starting product solution was diluted 1:40 with 39 g water. Viscosity: 0.95 mPa·s; Particle size distribution (PCS measurement): peak 1: d(H)=1370 nm−93.9 vol-%; peak 2: d(H)=4780 nm−6.1 vol-%.

### Synthesis:

Initiator solution was the same as for P-002, 1 wt.-% V-501 relative to monomers and crosslinking agent. Apparatus was the same as for P-002.455 g homogenized starting product solution was placed in the apparatus and heated to 90° C. When the temperature reached approximately 70° C., the apparatus was evacuated and flushed with nitrogen three times each to remove atmospheric oxygen (inhibitor). The apparatus was back-flushed with nitrogen to ensure pressure compensation over the entire polymerization. 25 g of initiator solution was then metered through a septum via a syringe, with good stirring. The reaction proceeded immediately. Due to the strongly exothermic reaction (5° C./min), cooling was provided immediately, and the reaction temperature was held constant at 90° C. After the initial exothermic conditions subsided, after 30 min the remaining 27 g initiator solution was added via syringe (no subsequent exothermic conditions were detectable). The reaction solution foamed over a period of approximately 30 min, but the foam subsequently disappeared.

### In-Process Control:

After a total reaction time of 4 h, 2 g reaction solution was withdrawn from the apparatus, placed on a tared aluminum dish, and dried in a drying oven at 180° C. for 30 min: Weigh-in=2.024 g of a low-viscosity white emulsion solution; weigh-out=0.609 g of a milky white, slightly tacky polymer. Solids content: 30.09% (TS_{100% theoretical conversion}: 31.50%)=95.5% conversion. Swelling behavior: Slightly dried polymer was combined with THF in a test tube; slight swelling was visually detectable. pH=8.5.

Reaction Solution:

The low-viscosity reaction solution having a bluish sheen was cooled to room temperature. 1 g reaction solution was diluted 1:40 with 39 g water. Viscosity: 1.33 mPa·s. Particle size distribution (PCS measurement): peak 1: d(H)=329 nm−100 vol-%; peak 2: No signal was present.

### Polymer Compound P-005

Polymer compound P-005, poly(2-acryloylamino-2-methylpropane sodium sulfonate-stat-2-ethylhexyl acrylate-stat-mPEG 1000 methacrylate-stat-N-(butoxymethyl)acrylamide), contains mPEG 1000 methacrylate as a further monomer in addition to 2-acryloylamino-2-methylpropane sodium sulfonate, 2-ethylhexyl acrylate, and N-(butoxymethyl)acrylamide. The mPEG 1000 methacrylate monomer is used to hydrophilize the polymer layer, thus enabling it to absorb water so that the negative charge sites in the matrix are more accessible.

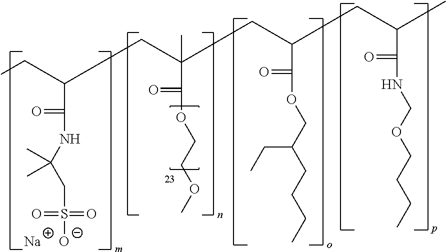

### Starting Product Solution:

| Weigh-in | | Product description |
|---|---|---|
| 30.0 | g | >98.0% 2-acryloylamino-2-methylpropanesulfonic acid, |
| | | Fluka |
| 120.0 | g | Plex 6969-O: mPEG 1000 methacrylate (contains 50% water |
| | | and 1-5% methacrylic acid), Evonik Industries, M = 1000 g/ |
| | | mol, d = 1.08 g/mL |
| 40.5 | g | 99% 2-ethylhexyl acrylate, stabilized, Acros Organics, |
| | | d = 0.880 g/mL, BP = 215° C. |
| 4.5 | g | Cylink NBMA monomer: 81% N-(butoxymethyl)acrylamide, |
| | | Cytec Industries |
| 4.5 | g | Disponil AFX 1080, Cognis Deutschland GmbH & Co. KG |
| 0.75 | g | Sodium dodecyl sulfate (SDS), p.a., Serva Feinbiochemika |
| 25.0 | g | Dipropylene glycol, d = 1.023 g/mL, BP = 90-95° C./ |
| | | 1 mm Hg |
| 21.0 | g | 50% sodium hydroxide (water content taken into account) |
| 194.0 | g | Deionized water |

### Synthesis:

Initiator solution was the same as for P-002, 1 wt.-% V-501 relative to monomers and crosslinking agent. Apparatus was the same as for P-002. Prehomogenization was the same as for P-002.455 g homogenized starting product solution was placed in the apparatus and heated to 90° C. When the temperature reached approximately 70° C., the apparatus was evacuated and flushed with nitrogen three times each to remove atmospheric oxygen (inhibitor). The apparatus was back-flushed with nitrogen to ensure pressure compensation over the entire polymerization. 25 g of initiator solution was then metered through a septum via a syringe, with good stirring. Only slightly exothermic conditions were detectable. The reaction temperature was held constant at 90° C. After 30 min the remaining 27 g initiator solution was added via syringe (no subsequent exothermic conditions were detectable), resulting in an emulsion having a bluish sheen. The reaction solution foamed over a period of approximately 30 min with an increase in viscosity, but the foam subsequently disappeared.

### In-Process Control:

After a total reaction time of 4 h, 2 g reaction solution was withdrawn from the apparatus, placed on a tared aluminum dish, and dried in a drying oven at 180° C. for 30 min: Weigh-in=2.185 g of a viscous white emulsion solution; weigh-out=0.746 g of a brownish, slightly tacky polymer; Solids content: 34.14% (TS_{100% theoretical conversion}: 31.8%)=107.3% (due to loss of solvent during the reaction, there was no conversion). Swelling behavior: Water was poured over slightly dried polymer, causing the polymer to swell to several times its original volume within minutes and become detached. Permanence appeared to be unsatisfactory. pH=6.5.

### Polymer Compound P-008

Polymer compound P-008, poly(2-acryloylamino-2-methylpropane sodium sulfonate-stat-2-ethylhexyl acrylate-stat-2-hydroxyethyl methacrylate-stat-N-(butoxymethyl)acrylamide), contains the monomers 2-acryloylamino-2-methylpropane sodium sulfonate, 2-hydroxyethyl methacrylate (HEMA), 2-ethylhexyl acrylate, and N-(butoxymethyl)acrylamide. HEMA is used to hydrophilize the polymer layer, thus enabling it to absorb water so that the negative charge sites in the matrix are more accessible.

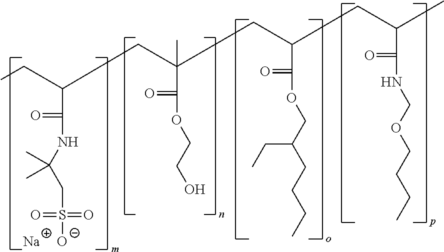

To avoid complete absorption of the solvent by the polymer having very high swelling capability, the starting product solution was diluted 1:4 with 2-propanol and water. The initiator solution was used undiluted in order to maintain the reaction rate without large losses.

### Starting Product Solution:

| Weigh-in | | Product description |
|---|---|---|
| 5.0 | g | >98.0% 2-acryloyl-2-methylpropanesulfonic acid, |
| | | Fluka |
| 6.7 | g | 95% 2-Hydroxyethyl methacrylate (HEMA), Fluka, |
| | | d = 1.07 g/mL, BP = 205-208° C. |
| 4.5 | g | 99% 2-ethylhexyl acrylate, stabilized, Acros Organics, |
| | | d = 0.880 g/mL, BP = 215° C. |
| 0.5 | g | Cylink NBMA monomer: 81% N-(butoxymethyl)acrylamide, |
| | | Cytec Industries |
| 10.0 | g | EP4 2-propanol, Schweizerhall, d = 0.785 g/mL, BP = 82° C. |
| 20.0 | g | Dipropylene glycol, d = 1.023 g/mL, BP = 90-95° C./ |
| | | 1 mm Hg |
| 2.0 | g | 50% sodium hydroxide (water content taken into account) |
| 12.0 | g | Deionized water |

| Weigh-in | Product description |
|---|---|
| 1.7 g | V-501: ≧98.0% 4,4′-azobis-(4-cyanovaleric acid), |
| | Fluka, M = 280.28 g/mol |
| 0.8 g | 50% sodium hydroxide |
| 67.5 g | Deionized water |

1/10 of the initiator solution was used, which corresponds to 1 wt.-% V-501 relative to monomers and crosslinking agent.

### Apparatus:

A 50-mL Schlenk tube with magnetic stirrer and septum was mounted on a magnetic stirrer heating plate having an aluminum heating block, and equipped with a temperature sensor. A discharge line with a stopcock on the Schlenk tube was used for evacuation and flushing with nitrogen.

### Synthesis:

10 g starting product solution (pH=5.5) was placed in the Schlenk tube and diluted 1:4 by adding 10 g 2-propanol and 20 g water. The Schlenk tube was then evacuated and flushed with nitrogen three times each to remove atmospheric oxygen (inhibitor). The Schlenk tube was back-flushed with nitrogen to ensure pressure compensation over the entire polymerization. The apparatus was then heated to 90° C., using a heating mantle. When the temperature reached approximately 70° C., 3.5 g of initiator solution was metered through the septum via a syringe, with good stirring. Only slightly exothermic conditions were detectable. The reaction temperature was held constant at 82° C. (boiling point of 2-propanol), resulting in a clear, whitish solution. After 30 min the remaining 3.5 g initiator solution was added via syringe (no subsequent exothermic conditions were detectable).

### In-Process Control:

After a total reaction time of 4 h, 8 g reaction solution was withdrawn from the apparatus, placed on a tared aluminum dish, and dried in a drying oven at 180° C. for 30 min: Weigh-in =8.033 g of a clear, bluish-white solution. Weigh-out=0.584 g of a clear, gold-brown, nontacky polymer. Solids content: 7.27% (TS_{100% theoretical conversion}: 6.40%)=113.6% (due to loss of solvent during the reaction, there was no conversion). Swelling behavior: Water was poured over slightly dried polymer, causing the polymer to swell slightly within minutes. According to visual observation, the polymer appeared to be only slightly crosslinked. pH=6.7.

### Polymer Compound P-009

Polymer compound P-009 was prepared analogously to P-008. The starting product solution was diluted only 1:2, using hexylene glycol (BP=197° C.) instead of 2-propanol, to increase the polymerization temperature to 90° C. The initiator solution was once again used undiluted in order to maintain the reaction rate without large losses.

### Synthesis:

Starting product solution, initiator solution, and apparatus were the same as for P-008.19 g starting product solution (pH=5.5) was placed in the Schlenk tube and diluted 1:2 by adding 19 g hexylene glycol. The Schlenk tube was then evacuated and flushed with nitrogen three times each to remove atmospheric oxygen (inhibitor). The Schlenk tube was back-flushed with nitrogen to ensure pressure compensation over the entire polymerization. The apparatus was then heated to 90° C., using a heating mantle. When the temperature reached approximately 70° C., 3.5 g of initiator solution was metered through the septum via a syringe, with good stirring. Only slightly exothermic conditions were detectable. The reaction temperature was held constant at 90° C. After 30 min the remaining 3.5 g initiator solution was added via syringe (no subsequent exothermic conditions were detectable), resulting in a clear, viscous solution.

### In-Process Control:

After a total reaction time of 4 h, 4 g reaction solution was withdrawn from the apparatus, placed on a tared aluminum dish, and dried in a drying oven at 180° C. for 30 min. Since the polymer was not yet dry due to the low-volatility hexylene glycol (BP=197° C.), the polymer was swelled with a small amount of water and dried in the drying oven at 180° C. for an additional 30 min. Weigh-in=4.093 g of a clear, bluish-white solution. Weigh-out=0.537 g of a clear, gold-brown, nontacky, brittle polymer. Solids content: 13.12% (TS_{100% theoretical conversion}: 12.36%)=106.1% (due to loss of solvent during the reaction, there was no conversion). Swelling behavior: Water was poured over slightly dried polymer, causing the polymer to immediately swell to several times its original volume and become detached. Permanence appeared to be unsatisfactory. pH=7.2.

### Polymer Compound P-010

Polymer compound P-010, poly(2-acryloylamino-2-methylpropane sodium sulfonate-stat-2-ethylhexyl acrylate-stat-mPEG 350 methacrylate-stat-N-(butoxymethyl)acrylamide), was prepared analogously to P-005, except that mPEG 350 methacrylate was used instead of mPEG 1000 methacrylate in order to investigate the effect of a shorter-chain mPEG monomer on permanence. 2-Propanol was added in addition to the emulsifiers in order to avoid phase separation of the starting product solution.

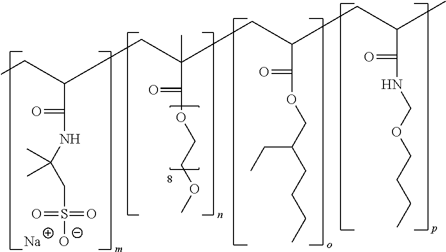

### Starting Product Solution:

| Weigh-in | | Product description |
|---|---|---|
| 2.48 | g | >98.0% 2-acryloylamino-2-methylpropanesulfonic acid, |
| | | Fluka |
| 3.30 | g | >95% mPEG 350 methacrylate, Evonik Industries, |
| | | M = 418 g/mol, d = 1.08 g/mL |
| 2.23 | g | 99% 2-ethylhexyl acrylate, stabilized, Acros Organics |
| 0.25 | g | Cylink NBMA monomer: 81% N-(butoxymethyl)acrylamide, |
| | | Cytec Industries |
| 0.25 | g | Disponil AFX 1080, Cognis Deutschland GmbH & Co. KG |
| 0.04 | g | Sodium dodecyl sulfate (SDS), p.a., Serva Feinbiochemika |
| 5.0 | g | 2-Propanol, Schweizerhall, M = 60.10 g/mol, d = 0.785 |
| | | g/mL, BP = 82° C. |
| 10.0 | g | Dipropylene glycol, d = 1.023 g/mL, BP = 90-95° C./ |
| | | 1 mm Hg |
| 1.0 | g | 50% sodium hydroxide (water content taken into account) |
| 6.0 | g | Deionized water |

### Prehomogenization:

The starting product solution was homogenized for two minutes in an ultrasonic bath.

### Synthesis:

Initiator solution and apparatus were the same as for P-008.30 g homogenized starting product solution was placed in the apparatus and heated to 80° C. When the temperature reached approximately 70° C., the apparatus was evacuated and flushed with nitrogen three times each to remove atmospheric oxygen (inhibitor). The apparatus was back-flushed with nitrogen to ensure pressure compensation over the entire polymerization. 1.8 g of initiator solution was then metered through the septum via a syringe, with good stirring. Only slightly exothermic conditions were detectable. The reaction temperature was held constant at 80° C. After 30 min the remaining approximately 1.7 g initiator solution was added via syringe (no subsequent exothermic conditions were detectable), resulting in an opaque, low-viscosity liquid.

### In-Process Control:

After a total reaction time of 4 h, 2 g reaction solution was withdrawn from the apparatus, placed on a tared aluminum dish, and dried in a drying oven at 180° C. for 30 min: Weigh-in =2.002 g of a viscous white emulsion solution; weigh-out=0.544 g of a milky white, nontacky polymer. Solids content: 27.17% (TS_{100 theoretical conversion}: 26.11%)=104.1% (due to loss of solvent during the reaction, there was no conversion). Swelling behavior: Water was poured over slightly dried polymer, causing the polymer to swell within minutes and become detached. Permanence appeared to be unsatisfactory. pH=8.1.

### Crosslinking Test Using Catalyst Stock Solution:

2 g reaction solution was added to a tared aluminum dish and diluted with 7 g water. 1 g catalyst stock solution (50 g/kg magnesium chloride×6H₂O+20 g/kg L-(+) tartaric acid) was added, and the mixture was dried in a drying oven at 180° C. for 30 min, resulting in a white, slightly brittle polymer. Swelling behavior: Water was poured over slightly dried polymer, causing the polymer to swell within minutes. The polymer was only moderately crosslinked.

### Summary:

The radical polymerization using mPEG 350 methacrylate instead of mPEG 1000 methacrylate (P-005) proceeded successfully. It was possible to synthesize a polymer having very good swelling capability within a very short time. However, the permanence properties of the polymer did not appear to be better than P-005.

### Polymer Compound P-011

Polymer compound P-011 was prepared analogously to P-010, but using 12% N-(butoxymethyl)acrylamide as crosslinking agent instead of 3% as before, to improve the permanence.

### Starting Product Solution:

| Weigh-in | | Product description |
|---|---|---|
| 2.48 | g | >98.0% 2-acryloylamino-2-methylpropanesulfonic acid, |
| | | Fluka |
| 3.30 | g | >95% mPEG 350 methacrylate, Evonik Industries |
| 1.49 | g | 99% 2-ethylhexyl acrylate, stabilized, Acros Organics |
| 0.99 | g | Cylink NBMA monomer: 81% N-(butoxymethyl)acrylamide, |
| | | Cytec Industries |
| 0.25 | g | Disponil AFX 1080, Cognis Deutschland GmbH & Co. KG |
| 0.04 | g | Sodium dodecyl sulfate (SDS), p.a., Serva Feinbiochemika |
| 5.0 | g | 2-Propanol, Schweizerhall, M = 60.10 g/mol, d = 0.785 |
| | | g/mL, BP = 82° C. |
| 10.0 | g | Dipropylene glycol, d = 1.023 g/mL, BP = 90-95° C./ |
| | | 1 mm Hg |
| 1.0 | g | 50% sodium hydroxide (water content taken into account) |
| 6.0 | g | Deionized water |

Initiator solution and apparatus were the same as for P-008. Prehomogenization, synthesis, and in-process control were the same as for P-010.

### Crosslinking Test Using Catalyst Stock Solution:

2 g reaction solution was added to a tared aluminum dish and diluted with 7 g water. 1 g catalyst stock solution (50 g/kg magnesium chloride×6H₂O+20 g/kg L-(+) tartaric acid) was added, and the mixture was dried in a drying oven at 180° C. for 30 min, resulting in a white, slightly brittle polymer. Swelling behavior: Water was poured over slightly dried polymer, causing the polymer to swell within minutes. According to visual observation, the polymer appeared to have fairly good crosslinking.

### Summary:

The radical polymerization using 12% N-(butoxymethyl)acrylamide as crosslinking agent instead of 3% (P-010) proceeded successfully. A crosslinking test using catalyst stock solution demonstrated that it was possible to synthesize a polymer having swelling capability within minutes, and fairly good crosslinking as determined visually.

### Polymer Compound P-012

P-012 is poly(2-acrylamido-2-methylpropane sodium sulfonate-stat-2-ethylhexyl acrylate-stat-2-hydroxyethyl methacrylate-stat-mPEG 350 methacrylate-stat-N-(butoxymethyl)acrylamide). The composition is analogous to P-010, except that it contains only 10% mPEG 350 methacrylate, and only 30% 2-hydroxyethyl methacrylate (HEMA). Since solution polymerization was carried out, no emulsifiers were used.

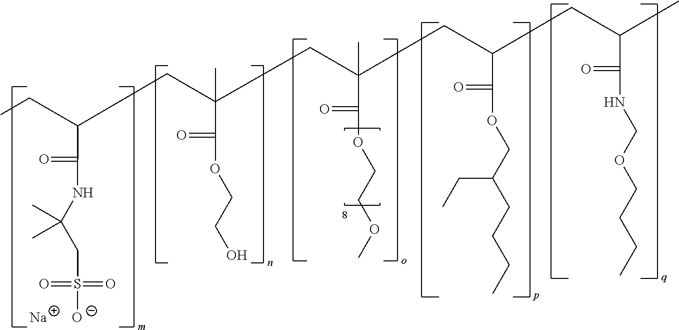

### Starting Product Solution:

| Weigh-in | | Product description |
|---|---|---|
| 2.48 | g | >98.0% 2-acryloylamino-2-methylpropanesulfonic acid, |
| | | Fluka |
| 0.83 | g | >95% mPEG 350 methacrylate, Evonik Industries |
| 2.48 | g | 97% 2-hydroxyethyl methacrylate (HEMA), stabilized, |
| | | Acros Organics, M = 130.15 g/mol, d = 1.07 g/mL, |
| | | BP = 205-208° C. |
| 2.23 | g | 99% 2-ethylhexyl acrylate, stabilized, Acros Organics |
| 0.25 | g | Cylink NBMA monomer: 81% N-(butoxymethyl)acrylamide, |
| | | Cytec Industries |
| 5.0 | g | 2-Propanol, Schweizerhall, M = 60.10 g/mol, d = 0.785 |
| | | g/mL, BP = 82° C. |
| 10.0 | g | Dipropylene glycol, d = 1.023 g/mL, BP = 90-95° C./ |
| | | 1 mm Hg |
| 1.0 | g | 50% sodium hydroxide (water content taken into account) |
| 16.0 | g | Deionized water |

Initiator solution and apparatus were the same as for P-008. Prehomogenization, synthesis, and in-process control were the same as for P-010. Swelling behavior: Water was poured over slightly dried polymer, causing the polymer to swell to several times its original volume and detach in clumps. The permanence appeared to be improved.

### Crosslinking Test Using Catalyst Stock Solution:

Same as for P-011, resulting in a white, slightly brittle polymer. Swelling behavior: Water was poured over slightly dried polymer, causing the polymer to swell within minutes. Due to its extreme brittleness as a result of the high HEMA fraction, the polymer did not have good permanence.

### Polymer Compound P-013

P-013, analogously to P-012, is poly(2-acrylamido-2-methylpropane sodium sulfonate-stat-2-ethylhexyl acrylate-stat-2-hydroxyethyl methacrylate-stat-mPEG 350 methacrylate-stat-N-(butoxymethyl)acrylamide). In addition, the crosslinking agent was increased to 12%. The swelling capability and crosslinking were once again investigated. Compared to P-012, the emulsifiers were once again used.

### Starting Product Solution:

| Weigh-in | | Product description |
|---|---|---|
| 2.48 | g | >98.0% 2-acryloylamino-2-methylpropanesulfonic |
| | | acid, Fluka |
| 0.83 | g | >95% mPEG 350 methacrylate, Evonik Industries, |
| | | M = 418 g/mol, d = 1.08 g/mL |
| 2.48 | g | 97% 2-hydroxyethyl methacrylate (HEMA), stabilized, |
| | | Acros Organics |
| 1.49 | g | 99% 2-ethylhexyl acrylate, stabilized, Acros Organics |
| 0.99 | g | Cylink NBMA monomer: 81% N-(butoxymethyl)acrylamide, |
| | | Cytec Industries |
| 0.25 | g | Disponil AFX 1080, Cognis Deutschland GmbH & Co. KG |
| 0.04 | g | Sodium dodecyl sulfate (SDS), p.a., Serva Feinbiochemika |
| 5.0 | g | 2-Propanol, Schweizerhall, M = 60.10 g/mol, d = 0.785 |
| | | g/mL, BP = 82° C. |
| 10.0 | g | Dipropylene glycol, d = 1.023 g/mL, BP = 90-95° C./ |
| | | 1 mm Hg |
| 1.0 | g | 50% sodium hydroxide (water content taken into account) |
| 6.0 | g | Deionized water |

Initiator solution and apparatus were the same as for P-008. Prehomogenization, synthesis, and in-process control were the same as for P-010. The reaction solution became increasingly more viscous over the course of the polymerization. The stirrer stopped, and a very tacky gel formed. The reaction was terminated.

### Summary:

Increasing the crosslinking agent content from 3% to 12% compared to P-012 increased the viscosity of the reaction solution in such a way that a very tacky, unstirrable gel formed. A test may be conducted to work without emulsifiers, i.e., to carry out a solution polymerization.

### Polymer Compound P-014

P-014 corresponds to P-013, except that it does not contain emulsifiers. Starting product solution: identical to P-013, but without emulsifiers (Disponil AFX 1080, sodium dodecyl sulfate). Initiator solution and apparatus were the same as for P-008. Prehomogenization, synthesis, and in-process control were the same as for P-010. The reaction solution became increasingly more viscous over the course of the polymerization. The stirrer stopped, and a very tacky gel formed. The reaction was terminated.

### Summary:

Despite omitting the emulsifiers compared to P-013, once again it was necessary to terminate the polymerization due to formation of a very tacky, unstirrable gel. Therefore, the polymerization had to be repeated, with dilution.

Polymer compound P-015

P-015 corresponds to P-014. The starting product solution was diluted 1:2 with water to prevent the polymer from forming a gel during the reaction. The initiator solution was used undiluted in order to maintain the reaction rate without large losses.

### Synthesis:

Starting product solution: Same as P-013, but without emulsifiers (Disponil AFX 1080, sodium dodecyl sulfate). Initiator solution and apparatus were the same as for P-008. Prehomogenization, synthesis, and in-process control were the same as for P-010.15 g homogenized starting product solution was placed in a Schlenk tube and diluted 1:2 by adding 15 g water. The 30 g of solution obtained was processed analogously to P-010. Swelling behavior: Water was poured over slightly dried polymer, causing the polymer to slowly swell and become detached. The permanence was poor, with the brittle polymer falling apart.

### Crosslinking Test Using Catalyst Stock Solution:

Same as P-011, resulting in a white, nontacky polymer. Swelling behavior: Water was poured over slightly dried polymer, causing the polymer to swell and become detached. The permanence was poor, with the granular (brittle) polymer falling apart.

### Summary:

Compared to P-013 and P-014, the polymer was easily prevented from forming a gel during the reaction by diluting the starting product solution 1:2 with water.

### Polymer Compound P-016

P-016 is based on P-015. To optimize the synthesis, the AMPS sodium salt was directly used instead of the sulfonic acid monomer. It was thus possible to dispense with the otherwise necessary neutralization of the starting product solution with sodium hydroxide.

### Starting Product Solution:

| Weigh-in | | Product description |
|---|---|---|
| 2.48 | g | AMPS Na 2403 monomer: 2-acrylamido-2-methylpropane |
| | | sodium sulfonate (contains approx. 50% water), Lubrizol, |
| | | M = 229.23 g/mol, d = 1.21 g/mL |
| 0.83 | g | >95% mPEG 350 methacrylate, Evonik Industries |
| 2.48 | g | 97% 2-hydroxyethyl methacrylate (HEMA), stabilized, |
| | | Acros Organics |
| 1.49 | g | 99% 2-ethylhexyl acrylate, stabilized, Acros Organics |
| 0.99 | g | Cylink NBMA monomer: 81% N-(butoxymethyl)acrylamide, |
| | | Cytec Industries |
| 5.0 | g | 2-Propanol, Schweizerhall, M = 60.10 g/mol, d = 0.785 g/mL, |
| | | BP = 82° C. |
| 10.0 | g | Dipropylene glycol, d = 1.023 g/mL, BP = 90-95° C./ |
| | | 1 mm Hg |
| 4.5 | g | Deionized water |

Initiator solution and apparatus were the same as for P-008. Prehomogenization, synthesis, and in-process control were the same as for P-010. Swelling behavior: Water was poured over slightly dried polymer, causing the polymer to slowly swell and become detached. The permanence was poor, with the brittle polymer falling apart.

### Crosslinking Test Using Catalyst Stock Solution:

Same as for P-011, resulting in a white, nontacky polymer. Swelling behavior: Water was poured over slightly dried polymer, causing the polymer to swell and become detached. The permanence was poor, with the granular (brittle) polymer falling apart.

### Summary:

The conversion and the properties of P-016 corresponded to those of P-015. Therefore, it was possible to dispense with the highly acidic AMPS (2-acrylamido-2-methylpropanesulfonic acid), which heretofore has always required neutralization with sodium hydroxide prior to the polymerization. Replacement with the AMPS sodium salt not only greatly reduces the level of effort, but also takes health protection of workers into account by dispensing with the highly corrosive, irritating AMPS.

### Polymer Compound P-017

P-017 is a repetition of P-016, except with V-50 as initiator and at a reaction temperature of 70° C.

### Initiator Solution:

| Weigh-in | Product description |
|---|---|
| 0.83 g | V-50, 2,2′-azobis-(2-amidinopropane) dihydrochloride, |
| | Wako, M = 271.19 g/mol, t_{1/2} = 35 min at 80° C. |
| 0.8 g | 50% sodium hydroxide |
| 34.2 g | Deionized water |

1/10 of the initiator solution was used, which corresponds to 1 wt.-% V-50 relative to monomers and crosslinking agent.

Starting product solution was the same as for P-016. Apparatus was the same as for P-008. Prehomogenization, synthesis, and in-process control were the same as for P-010. Swelling behavior: Water was poured over slightly dried polymer, causing the polymer to slowly swell and become detached. The permanence was poor, with the brittle polymer falling apart.

### Crosslinking Test Using Catalyst Stock Solution:

Same as P-011. A white, nontacky polymer was obtained. Swelling behavior: Water was poured over slightly dried polymer, causing the polymer to swell and become detached. The permanence was poor, with the granular (brittle) polymer falling apart.

### Summary:

The conversion and the properties of P-017 corresponded to those of P-015 and P-016. That is, the replacement of initiator V-501 (4,4′-azobis-(4-cyanovaleric acid) with V-50 (2,2-azobis-(2-amidinopropane)dihydrochloride) further optimized the polymerization conditions, since it was possible to dispense with the previous neutralization of V-501 with sodium hydroxide to increase the solubility in water. V-50 is not only readily soluble in water, but also has a lower half-life at the corresponding temperature. Therefore, at the same conversion, the polymerization temperature may be reduced from the initial 90° C. or 80° C. to approximately 70° C. The cationic character of V-50 apparently has no adverse effect on the polymerization.

P-018 through P-026: Screening with Different Fractions of mPEG 350 Methacrylate and HEMA, using 0% Crosslinking Agent

Different variants of poly(2-acrylamido-2-methylpropane sodium sulfonate-stat-2-ethylhexyl acrylate-stat-2-hydroxyethyl methacrylate-stat-mPEG 350 methacrylate-stat-N-(butoxymethyl)acrylamide) polymer compounds were tested, using 0% crosslinking agent.

In order for the anionic charge of the polymers to be accessible to the cationic emulsion during the loading process, the donor layer must have good swelling capability in an aqueous environment. Good short-term swelling capability is important, in particular for washing machine applications. The swelling capability, the same as the permanence, is influenced by the crosslinking rate. The composition of the polymers according to the invention was optimized using screening tests. Based on P-017, the polymers had the following base composition: 30 wt.-% AMPS sodium salt, 30 wt.-% 2-ethylhexyl acrylate, variable 0-40 wt.-% mPEG 350 methacrylate and 40-0 wt.-% 2-hydroxyethyl methacrylate (HEMA). The polymerizations were carried out at a reaction temperature of 70° C., using V-50 as initiator.

### Starting Product Solution:

| Weigh-in | | Product description |
|---|---|---|
| 24.8 | g | AMPS Na 2403 monomer: 2-acrylamido-2-methylpropane |
| | | sodium sulfonate |
| 0-16.5 | g | >95% mPEG 350 methacrylate, Evonik Industries |
| 16.5-0 | g | 97% 2-hydroxyethyl methacrylate (HEMA), stabilized, |
| | | Acros Organics |
| 12.4 | g | 99% 2-ethylhexyl acrylate, stabilized, Acros Organics |
| 0 | g | Cylink NBMA monomer: 81% N-(butoxymethyl)acryl- |
| | | amide, Cytec Industries |
| 25.0 | g | 2-Propanol, Schweizerhall, M = 60.10 g/mol, d = 0.785 |
| | | g/mL, BP = 82° C. |
| 50.0 | g | Dipropylene glycol, d = 1.023 g/mL, BP = 90-95° C./ |
| | | 1 mm Hg |
| 22.5 | g | Deionized water |

| | | | Swelling rate |
|---|---|---|---|
| | | | (wet weight |
| Polymer | mPEG 350 | | divided by |
| compound | methacrylate | HEMA | dry weight) |
| P-018 | 0 wt.-%; 0 g | 40 wt.-%; 1.65 g | 78% |
| P-019 | 5 wt.-%; 0.21 g | 35 wt.-%; 1.44 g | 72% |
| P-020 | 10 wt.-%; 0.41 g | 30 wt.-%; 1.24 g | 250% |
| P-021 | 15 wt.-%; 0.62 g | 25 wt.-%; 1.03 g | 772% |
| P-022 | 20 wt.-%; 0.83 g | 20 wt.-%; 0.83 g | 864% |
| P-023 | 25 wt.-%; 1.03 g | 15 wt.-%; 0.62 g | 811% |
| P-024 | 30 wt.-%; 1.24 g | 10 wt.-%; 0.41 g | 614% |
| P-025 | 35 wt.-%; 1.44 g | 5 wt.-%; 0.21 g | 332% |
| P-026 | 40 wt.-%; 1.65 g | 0 wt-.%; 0 g | 176% |

Apparatus was the same as for P-008. Prehomogenization was the same as for P-010. Initiator solution, synthesis, and in-process control were the same as for P-017. The solids content was between 12.3% and 16.5% for all polymers. The polymers varied from milky white (P-018) to gold-brown (P-026). The brittleness of polymers P-018 through P-026 did not vary. None of the polymers were friable. The tackiness of the polymers varied from slightly tacky (P-018) to tacky (P-026). No clear tendency regarding swelling capability of the polymers was visually apparent. The higher the HEMA fraction (P-018), the greater the swelling of the polymers over the entire surface, whereas for the polymers containing higher mPEG 350 methacrylate fractions (P-026), the swelling began from the side of the polymer surface. The permanence was very poor for all polymers (0% crosslinking agent). However, some of the polymers appeared to hold together somewhat better. This could be due to an mPEG 350 dimethacrylate impurity, which is always present as a by-product in the mPEG 350 methacrylate monomers.

### Swelling Behavior:

The dried polymers in the aluminum dishes (in-process control) were covered with water that was not deionized, and stored at room temperature for 24 h. The swelled polymers were then centrifuged out at 1000 rpm via a tared filter syringe (plastic syringe having a plastic mesh used as a filter) for 2 min, thus separating the supernatant water. The wet weight of the polymers was determined by reweighing the filter syringe.

### Summary:

The radical polymerizations proceeded successfully. Due to solvent losses during the reaction, the solids contents (in-process control) in each case were higher than the theoretically possible conversion. For this reason, each reaction solution was subsequently diluted with water to a polymer concentration of 12.0%. It was thus possible to synthesize polymers having very good swelling capability within a very short time. The permanence properties of screening series P-018 through P-026 were very poor, as expected, since the polymers contained 0% NBMA crosslinking agent. Thus, determination of the swelling rate by centrifuging in the P-0,8-screening series was not particularly meaningful, since the polymers containing 0% NBMA were not crosslinkable, or were crosslinkable only in an undefined manner, in the drying oven (in-process control), and therefore the suitability of the filter syringe varied. Nevertheless, the determination of the swelling rates showed a swelling capability of approximately 800% and higher for some polymers.

P-027 through P-035: Screening with Different Fractions of mPEG 350 Methacrylate and HEMA, using 5% Crosslinking Agent

Different variants of poly(2-acrylamido-2-methylpropane sodium sulfonate-stat-2-ethylhexyl acrylate-stat-2-hydroxyethyl methacrylate-stat-mPEG 350 methacrylate-stat-N-(butoxymethyl)acrylamide) polymer compounds were tested, using 5% crosslinking agent.

Based on P-017, the polymers had the following base composition: 30 wt.-% AMPS sodium salt, 5 wt.-% NBMA crosslinking agent, 25 wt.-% 2-ethylhexyl acrylate, variable 0 to 40 wt.-% mPEG 350 methacrylate and 40 to 0 wt.-% 2-hydroxyethyl methacrylate (HEMA). The polymerizations were carried out at a reaction temperature of 70° C., using V-50 as initiator.

### Starting Product Solution:

| Weigh-in | | Product description |
|---|---|---|
| 24.8 | g | AMPS Na 2403 monomer: 2-acrylamido-2-methylpropane |
| | | sodium sulfonate |
| 0-16.5 | g | >95% mPEG 350 methacrylate, Evonik Industries |
| 16.5-0 | g | 97% 2-hydroxyethyl methacrylate (HEMA), stabilized, |
| | | Acros Organics |
| 10.3 | g | 99% 2-ethylhexyl acrylate, stabilized, Acros Organics |
| 2.1 | g | Cylink NBMA monomer: 81% N-(butoxymethyl)acryl- |
| | | amide, Cytec Industries |
| 25.0 | g | 2-Proganol, Schweizerhall, M = 60.10 g/mol, d = 0.785 |
| | | g/mL, BP = 82° C. |
| 50.0 | g | Dipropylene glycol, d = 1.023 g/mL, BP = 90-95° C./ |
| | | 1 mm Hg |
| 22.5 | g | Deionized water |

| | | | Swelling rate |
|---|---|---|---|
| | | | (wet weight |
| | mPEG 350 | | divided by |
| Polymer | methacrylate | HEMA | dry weight) |
| P-027 | 0 wt.-%; 0 g | 40 wt.-%; 1.65 g | 406% |
| P-028 | 5 wt.-%; 0.21 g | 35 wt.-%; 1.44 g | 400% |
| P-029 | 10 wt.-%; 0.41 g | 30 wt.-%; 1.24 g | 381% |
| P-030 | 15 wt.-%; 0.62 g | 25 wt.-%; 1.03 g | 375% |
| P-031 | 20 wt.-%; 0.83 g | 20 wt.-%; 0.83 g | 361% |
| P-032 | 25 wt.-%; 1.03 g | 15 wt.-%; 0.62 g | 336% |
| P-033 | 30 wt.-%; 1.24 g | 10 wt.-%; 0.41 g | 343% |
| P-034 | 35 wt.-%; 1.44 g | 5 wt.-%; 0.21 g | 345% |
| P-035 | 40 wt.-%; 1.65 g | 0 wt.-%; 0 g | 363% |

The apparatus, test procedure. synthesis, swelling tests, etc. were analogous to P-018 through P-026. The solids content was between 12.5% and 16.5% for all polymers. The polymers varied from light gold-brown (P-027) to gold-brown (P-035). The brittleness of the polymers varied from very slightly brittle (P-027) to non-brittle (P-035). The tackiness of the polymers varied from very slightly tacky (P-027) to slightly tacky (P-035). No clear tendency regarding swelling capability of the polymers was visually apparent. The higher the HEMA fraction (P-027), the greater the swelling of the polymers over the entire surface, whereas for the polymers containing higher mPEG 350 methacrylate fractions (P-035), the swelling began from the side of the polymer surface.

### Summary:

The radical polymerizations proceeded successfully. After 24 h at room temperature, the swelling rates of screening series P-027 through P-035, using 5% NBMA crosslinking agent, were between 336% and 406%.

P-036 through P-044: Screening with Different Fractions of mPEG 350 Methacrylate and HEMA, using 10% Crosslinking Agent

Different variants of poly(2-acrylamido-2-methylpropane sodium sulfonate-stat-2-ethylhexyl acrylate-stat-2-hydroxyethyl methacrylate-stat-mPEG 350 methacrylate-stat-N-(butoxymethyl)acrylamide) polymer compounds were tested, using 10% crosslinking agent.

Based on P-017, the polymers had the following base composition: 30 wt.-% AMPS sodium salt, 10 wt.-% NBMA crosslinking agent, 20 wt.-% 2-ethylhexyl acrylate, variable 0 to 40 wt.-% mPEG 350 methacrylate and 40 to 0 wt.-% 2-hydroxyethyl methacrylate (HEMA). The polymerizations were carried out at a reaction temperature of 70° C., using V-50 as initiator.

### Starting Product Solution:

| Weigh-in | | Product description |
|---|---|---|
| 24.8 | g | AMPS Na 2403 monomer: 2-acrylamido-2-methylpropane |
| | | sodium sulfonate |
| 0-16.5 | g | >95% mPEG 350 methacrylate, Evonik Industries |
| 16.5-0 | g | 97% 2-hydroxyethyl methacrylate (HEMA), stabilized, |
| | | Acros Organics |
| 8.3 | g | 99% 2-ethylhexyl acrylate, stabilized, Acros Organics |
| 4.1 | g | Cylink NBMA monomer: 81% N-(butoxymethyl)acryl- |
| | | amide, Cytec Industries |
| 25.0 | g | 2-Propanol, Schweizerhall, M = 60.10 g/mol, d = 0.785 |
| | | g/mL, BP = 82° C. |
| 50.0 | g | Dipropylene glycol, d = 1.023 g/mL, BP = 90-95° C./ |
| | | 1 mm Hg |
| 22.5 | g | Deionized water |

| | | | Swelling rate |
|---|---|---|---|
| | | | (wet weight |
| | mPEG 350 | | divided by |
| Polymer | methacrylate | HEMA | dry weight) |
| P-036 | 0 wt.-%; 0 g | 40 wt.-%; 1.65 g | 261% |
| P-037 | 5 wt.-%; 0.21 g | 35 wt.-%; 1.44 g | 253% |
| P-038 | 10 wt.-%; 0.41 g | 30 wt.-%; 1.24 g | 242% |
| P-039 | 15 wt.-%; 0.62 g | 25 wt.-%; 1.03 g | 224% |
| P-040 | 20 wt.-%; 0.83 g | 20 wt.-%; 0.83 g | 238% |
| P-041 | 25 wt.-%; 1.03 g | 15 wt.-%; 0.62 g | 237% |
| P-042 | 30 wt.-%; 1.24 g | 10 wt.-%; 0.41 g | 242% |
| P-043 | 35 wt.-%; 1.44 g | 5 wt.-%; 0.21 g | 265% |
| P-044 | 40 wt.-%; 1 .65 g | 0 wt.-%; 0 g | 278% |

The apparatus, test procedure, synthesis, swelling tests, etc. were analogous to P-018 through P-026. The solids content was between 12.2% and 14.3% for all polymers. The polymers varied from light gold-brown (P-036) to gold-brown (P-044). The brittleness of the polymers varied from slightly brittle (P-036) to non-brittle (P-044). The tackiness of the polymers did not vary from P-036 to P-044. None of the polymers was tacky. No clear tendency regarding swelling capability of the polymers was visually apparent. The higher the HEMA fraction (P-036), the greater the swelling of the polymers over the entire surface, whereas for the polymers containing higher mPEG 350 methacrylate fractions (P-044), the swelling began from the side of the polymer surface.

### Summary:

The radical polymerizations proceeded successfully. After 24 h at room temperature, the swelling rates of screening series P-036 through P-044, using 10% NBMA crosslinking agent, were between 224% and 278%.

P-045 through P-053: Screening with Different Fractions of mPEG 350 Methacrylate and HEMA, using 15% Crosslinking Agent

Different variants of poly(2-acrylamido-2-methylpropane sodium sulfonate-stat-2-ethylhexyl acrylate-stat-2-hydroxyethyl methacrylate-stat-mPEG 350 methacrylate-stat-N-(butoxymethyl)acrylamide) polymer compounds were tested, using 15% crosslinking agent.

Based on P-017, the polymers had the following base composition: 30 wt.-% AMPS sodium salt, 15 wt.-% NBMA crosslinking agent, 15 wt.-% 2-ethylhexyl acrylate, variable 0 to 40 wt.-% mPEG 350 methacrylate and 40 to 0 wt.-% 2-hydroxyethyl methacrylate (HEMA). The polymerizations were carried out at a reaction temperature of 70° C., using V-50 as initiator.

### Starting Product Solution:

| Weigh-in | | Product description |
|---|---|---|
| 24.8 | g | AMPS Na 2403 monomer: 2-acrylamido-2-methylpropane |
| | | sodium sulfonate |
| 0-16.5 | g | >95% mPEG 350 methacrylate, Evonik Industries |
| 16.5-0 | g | 97% 2-hydroxyethyl methacrylate (HEMA), stabilized, |
| | | Acros Organics |
| 6.2 | g | 99% 2-ethylhexyl acrylate, stabilized, Acros Organics |
| 6.2 | g | Cylink NBMA monomer: 81% N-(butoxymethyl)acryl- |
| | | amide, Cytec Industries |
| 25.0 | g | 2-Propanol, Schweizerhall, M = 60.10 g/mol, d = 0.785 |
| | | g/mL, BP = 82° C. |
| 50.0 | g | Dipropylene glycol, d = 1.023 g/mL, BP = 90-95° C./ |
| | | 1 mm Hg |
| 22.5 | g | Deionized water |

| | | | Swelling rate |
|---|---|---|---|
| | | | (wet weight |
| | mPEG 350 | | divided by |
| Polymer | methacrylate | HEMA | dry weight) |
| P-045 | 0 wt.-%; 0 g | 40 wt.-%; 1.65 g | 219% |
| P-046 | 5 wt.-%; 0.21 g | 35 wt.-%; 1.44 g | 216% |
| P-047 | 10 wt.-%; 0.41 g | 30 wt.-%; 1.24 g | 205% |
| P-048 | 15 wt.-%; 0.62 g | 25 wt.-%; 1.03 g | 201% |
| P-049 | 20 wt.-%; 0.83 g | 20 wt.-%; 0.83 g | 206% |
| P-050 | 25 wt.-%; 1.03 g | 15 wt.-%; 0.62 g | 224% |
| P-051 | 30 wt.-%; 1.24 g | 10 wt.-%; 0.41 g | 226% |
| P-052 | 35 wt.-%; 1.44 g | 5 wt.-%; 0.21 g | 239% |
| P-053 | 40 wt.-%; 1.65 g | 0 wt.-%; 0 g | 269% |

The apparatus, test procedure, synthesis, swelling tests, etc. were analogous to P-018 through P-026. The solids content was between 12.6% and 17.0% for all polymers. The polymers varied from light gold-brown (P-045) to gold-brown (P-053). The brittleness of the polymers varied from brittle (P-045) to slightly brittle (P-053). The tackiness of the polymers did not vary from P-045 to P-053. None of the polymers was tacky. No clear tendency regarding swelling capability of the polymers was visually apparent. The higher the HEMA fraction (P-045), the greater the swelling of the polymers over the entire surface, whereas for the polymers containing higher mPEG 350 methacrylate fractions (P-053), the swelling began from the side of the polymer surface.

### Summary:

The radical polymerizations proceeded successfully. After 24 h at room temperature, the swelling rates of screening series P-045 through P-053, using 15% NBMA crosslinking agent, were between 201% and 269%.

### Polymer Compound P-054

Screening tests P-018 through P-053 were carried out with variable contents of mPEG 350 methacrylate and 2-hydroxyethyl methacrylate (HEMA), and different contents of NBMA crosslinking agent, resulting in good swelling capability for all polymers. Although the wash permanence of the PA fabric impregnated with the polymers was rather unsatisfactory, a loss in permanence of approximately 80% was constant over the entire screening. This was independent of the NBMA crosslinking agent contents of 5%, 10%, and 15%, with the exception of 0% NBMA crosslinking agent (very poor permanence).

Surprisingly, however, it turned out that the polymers containing 30% and 35% mPEG 350 methacrylate, and 10% and 5% 2-hydroxyethyl methacrylate (HEMA), respectively, had the best accessibility to the anionic charge, up to a crosslinking agent concentration of 10%. This is due to the fact that at higher HEMA contents the network density increases, since the crosslinking agent molecules are able to crosslink with HEMA.

Based on the composition of P-042, the polymerization was repeated using only 1% NBMA crosslinking agent. The purpose was to investigate whether in particular the wash permanence, contrary to expectations, was better than in the screening tests using 5%, 10%, and 15% NBMA crosslinking agent. Preliminary tests showed that at the 1:2 dilution of the starting product solution with water, a phase separation occurred which could not be eliminated even by the use of 3.0% Disponil AFX 1080 and 0.5% sodium dodecyl sulfate (SDS). Therefore, the polymerization was carried out undiluted.

### Starting Product Solution:

| Weigh-in | Product description |
|---|---|
| 4.96 g | AMPS Na 2403 monomer: 2-acrylamido-2-methylpropane |
| | sodium sulfonate |
| 2.48 g | >95% mPEG 350 methacrylate, Evonik Industries |
| 0.83 g | 97% 2-hydroxyethyl methacrylate (HEMA), stabilized, |
| | Acros Organics |
| 2.4 g | 99% 2-ethylhexyl acrylate, stabilized, Acros Organics |
| 0.08 g | Cylink NBMA monomer: 81% N-(butoxymethyl)acryl- |
| | amide, Cytec Industries |
| 5.0 g | 2-Propanol, Schweizerhall, M = 60.10 g/mol, d = 0.785 |
| | g/mL, BP = 82° C. |
| 10.0 g | Dipropylene glycol, d = 1.023 g/mL, BP = 90-95° C./ |
| | 1 mm Hg |
| 4.5 g | Deionized water |

The apparatus, test procedure, synthesis, swelling tests, etc. were analogous to P-018 through P-026. Initiator solution was the same as for P-017.

### Summary:

The radical polymerization using only 1% NBMA crosslinking agent proceeded successfully. After a reaction time of 4 h, the conversion was 99.0%. To simplify the subsequent finishing tests, the reaction solution was diluted with water to a polymer concentration of 12.0%. It was possible to synthesize a polymer having very good swelling capability within a very short time. Although the polymer fell apart in rough pieces, according to visual observation the permanence appeared better.

### Polymer Compound P-055

Based on the composition of P-042, NMA crosslinking monomer (N-(methylol)acrylamide) was used as crosslinker for the test instead of N-(butoxymethyl)acrylamide (NBMA) in order to improve the elasticity of the donor layer during swelling, with the aim of increasing the layer permanence due to fewer stress fractures. The polymerization was carried out in a Schlenk tube at a reaction temperature of 70° C., using V-50 as initiator. To allow the actual permanence to be assessed, NMA was used in equimolar quantities relative to NBMA (P-042). Preliminary tests showed that at the 1:2 dilution of the starting product solution with water, a phase separation occurred which could not be eliminated even by the use of 3.0% Disponil AFX 1080 and 0.5% sodium dodecyl sulfate (SDS). Therefore, the polymerization was carried out undiluted.

### Starting Product Solution:

| Weigh-in | Product description |
|---|---|
| 4.96 g | AMPS Na 2403 monomer: 2-acrylamido-2-methylpropane |
| | sodium sulfonate |
| 2.48 g | >95% mPEG 350 methacrylate, Evonik Industries |
| 0.83 g | 97% 2-hydroxyethyl methacrylate (HEMA), stabilized, |
| | Acros Organics |
| 1.65 g | 99% 2-ethylhexyl acrylate, stabilized, Acros Organics |
| 0.91 g, | Cylink NMA monomer: 48% N-(methylol)acrylamide in |
| 4.33 mmol | water, Cytec Industries, M = 101.10 g/mol, |
| | d = 1.074 g/mL, BP = 100° C. (water) |
| 5.0 g | 2-Propanol, Schweizerhall, M = 60.10 g/mol, d = 0.785 |
| | g/mL, BP = 82° C. |
| 10.0 g | Dipropylene glycol, d = 1.023 g/mL, BP = 90-95° C./ |
| | 1 mm Hg |
| 4.42 g | Deionized water |

The apparatus, test procedure, synthesis, swelling tests, etc. were analogous to P-054. Initiator solution was the same as for P-017.

### Summary:

The radical polymerization using the NMA crosslinking monomer (N-(methylol)acrylamide) instead of N-(butoxymethyl)acrylamide (NBMA) proceeded successfully. To simplify the subsequent finishing tests, the reaction solution was diluted with water to a polymer concentration of 12.0%. It was possible to synthesize a polymer having very good swelling capability within a very short time.

### Polymer Compound P-056

Based on the composition of P-042, glycidyl methacrylate (GMA) crosslinking monomer was used as crosslinker for the test instead of N-(butoxymethyl)acrylamide (NBMA) in order to improve the elasticity of the donor layer during swelling, with the aim of increasing the layer permanence due to fewer stress fractures. To allow assessment of the actual permanence, the crosslinking monomer was used in equimolar quantities relative to NBMA (P-042).

### Starting Product Solution:

| Weigh-in | Product description |
|---|---|
| 4.96 g | AMPS Na 2403 monomer: 2-acrylamido-2-methylpropane |
| | sodium sulfonate |
| 2.48 g | >95% mPEG 350 methacrylate, Evonik Industries |
| 0.83 g | 97% 2-hydroxyethyl methacrylate (HEMA), stabilized, |
| | Acros Orqanics |
| 0.62 g | 99% 2-ethylhexyl acrylate, stabilized, Acros Organics, |
| | d = 0.880 g/mL, BP = 215° C. |
| 0.91 g, | ≧97% glycidyl methacrylate (GMA), Fluka, M = 142.15 |
| 4.33 mmol | g/mol, d = 1.075 g/mL, BP = 192-197° C. |
| 5.0 g | 2-Propanol, Schweizerhall, M = 60.10 g/mol, d = 0.785 g/mL, |
| | BP = 82° C. |
| 10.0 g | Dipropylene glycol, d = 1.023 g/mL, BP = 90-95° C./ |
| | 1 mm Hg |
| 4.71 g | Deionized water |

The apparatus, test procedure, synthesis, swelling tests, etc. were analogous to P-054. Initiator solution was the same as for P-017.

### Summary:

The radical polymerization using GMA proceeded successfully. To simplify the subsequent finishing tests, the reaction solution was diluted with water to a polymer concentration of 12.0%. It was possible to synthesize a polymer having very good swelling capability within a very short time.

### Polymer Compound P-057

Based on the composition of P-042, p-EMKO-TDI-o-HEMA crosslinking monomer was used for the test instead of N-(butoxymethyl)acrylamide (NBMA). p-EMKO-TDI-o-HEMA has a longer chain than the crosslinking agents NBMA, NMA, and GMA. The elasticity of the donor layer during swelling is increased due to the resulting greater distance between the polymer chains after crosslinking. The aim was to thus decrease the stress fractures in the polymer during swelling, thereby increasing the layer permanence. To allow assessment of the actual permanence, the crosslinking monomer was used in equimolar quantities relative to NBMA (P-042). Preliminary tests showed that gel formation occurred for the undiluted polymerization. To avoid this while still obtaining a monophase starting product solution, the starting product solution was diluted 1:2 with 2-propanol instead of water. The initiator solution was used undiluted in order to maintain the reaction rate without large losses.

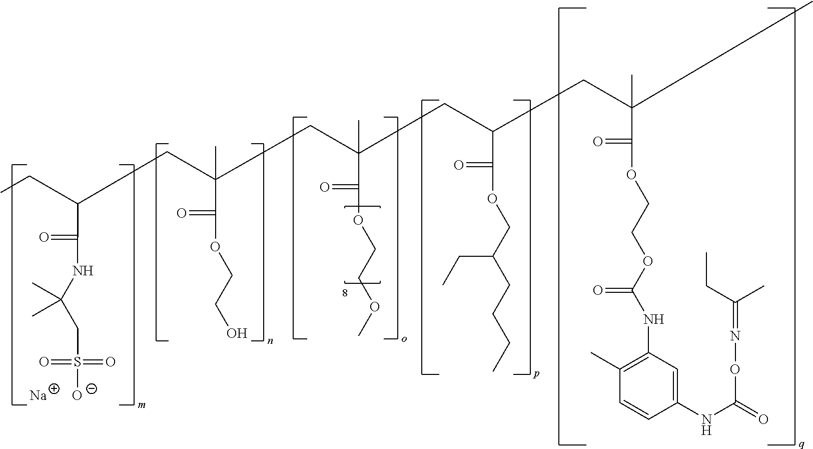

### Starting Product Solution:

| Weigh-in | Product description |
|---|---|
| 4.96 g | AMPS Na 2403 monomer: 2-acrylamido-2-methylpropane |
| | sodium sulfonate |
| 2.48 g | >95% mPEG 350 methacrylate, Evonik Industries |
| 0.83 g | 97% 2-hydroxyethyl methacrylate (HEMA), stabilized, |
| | Acros Organics |
| 1.65 g | 99% 2-ethylhexyl acrylate, stabilized, Acros Organics |
| 1.69 g, | p-EMKO-TDI-o-HEMA, pure according to FT-IR, M = |
| 4.33 mmol | 391.42 g/mol |
| 5.0 g | 2-Propanol, Schweizerhall, M = 60.10 g/mol, d = 0.785 |
| | g/mL, BP = 82° C. |
| 10.0 g | Dipropylene glycol, d = 1.023 g/mL, BP = 90-95° C./ |
| | 1 mm Hg |
| 3.64 g | Deionized water |

The apparatus, test procedure, synthesis, swelling tests, etc. were analogous to P-054. Initiator solution was the same as for P-017.

### Synthesis of the Crosslinking Monomer:

The difference in reactivities of the two isocyanate groups of 2,4-toluylene diisocyanate provides a pathway for a crosslinking monomer, in that in a first reaction step the isocyanate group in the p-position is selectively blocked, using an EMKO protective group, and then in a second reaction step the remaining isocyanate group is reacted with the radically polymerizable 2-hydroxyethyl methacrylate unit. It is absolutely necessary to use dry heptane as solvent so that the p-EMKO-TDI adduct from step 1 does not “oil out,” but instead, crystalline needles precipitate. The monoadduct is thus removed from the reaction so that the second NCO group of 2,4-TDI is also not able to react with EMKO (selectivity >93% according to NMR). It is necessary to use DABCO as catalyst in order to increase the reactivity of the OH groups of HEMA (OH groups are approximately 4000 times less nucleophilic than amines). Literature reference: Duschek. G. K., Partially fluorinated and reactive polymers for the oil-repellent surface modification of cotton and cellulose, Dissertation, University of Ulm, 1997, 64-69/178.

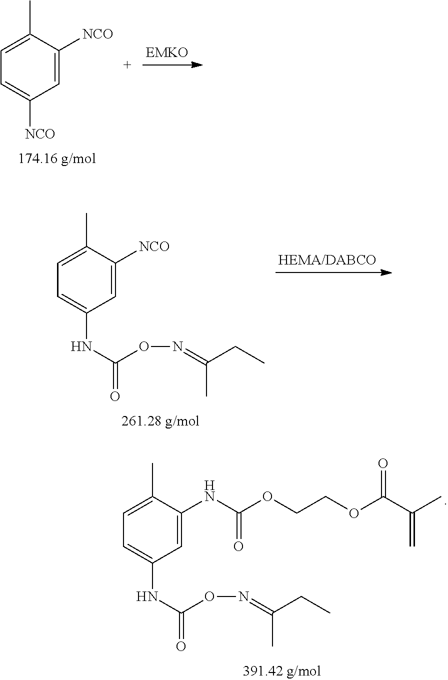

### Summary:

The radical polymerization using p-EMKO-TDI-o-HEMA crosslinking monomer proceeded successfully. It was possible to synthesize a polymer having very good swelling capability within a very short time.

### Polymer Compound P-058

Based on the composition of P-042, EMKO-2-(N-(tert-butyl){[(3-isocyanato-1,5,5-trimethylcyclohexyl)methyl]amino}-carbonylamino)ethyl methacrylate was used as crosslinking monomer instead of NBMA. EMKO-2-(N-(tert-butyl){[(3-isocyanato-1,5,5-trimethylcyclohexyl)methyl]amino}carbonylamino)ethyl methacrylate has a longer chain than the crosslinking agents NBMA, NMA, and GMA. The elasticity of the donor layer during swelling is increased due to the resulting greater distance between the polymer chains after crosslinking. The aim was to thus decrease the stress fractures in the polymer during swelling, thereby increasing the layer permanence. To allow assessment of the actual permanence, the crosslinking monomer was used in equimolar quantities relative to NBMA (P-042). Preliminary tests showed that gel formation occurred for the undiluted polymerization. To avoid this while still obtaining a monophase starting product solution, the starting product solution was diluted 1:2 with 2-propanol instead of water. The initiator solution was used undiluted in order to maintain the reaction rate without large losses.

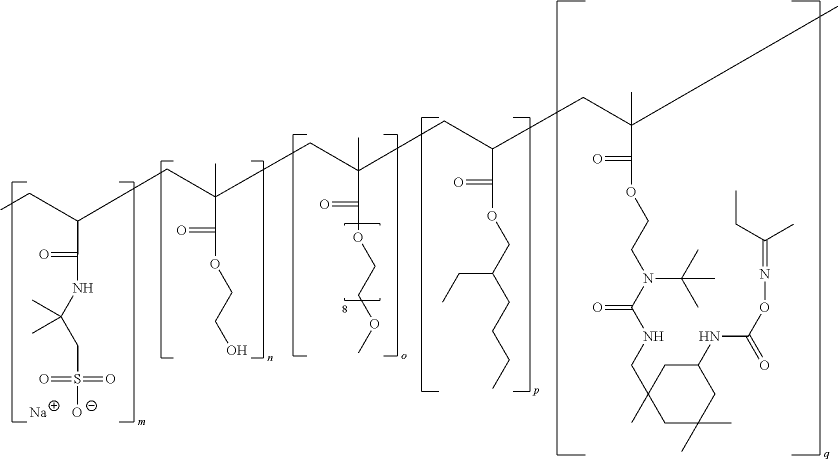

### Starting Product Solution:

| Weigh-in | Product description |
|---|---|
| 4.96 g | AMPS Na 2403 monomer: 2-acrylamido-2-methylpropane |
| | sodium sulfonate |
| 2.48 g | >95% mPEG 350 methacrylate, Evonik Industries |
| 0.83 g | 97% 2-hydroxyethyl methacrylate (HEMA), stabilized, |
| | Acros Organics |
| 1.65 g | 99% 2-ethylhexyl acrylate, stabilized, Acros Organics |
| 2.14 g, | EMKO alkenyl isocyanate: EMKO-2-(N-(tert- |
| 4.33 mmol | butyl){[(3-isocyanato-1,5,5-trimethyl- |
| | cyclohexyl)methyl]amino}carbonylamino)ethyl |
| | methacrylate, pure according to FT-IR, M = 494.67 g/mol |
| 5.0 g | 2-Propanol, Schweizerhall, M = 60.10 g/mol, |
| | d = 0.785 g/mL, BP = 82° C. |
| 10.0 g | Dipropylene glycol, d = 1.023 g/mL, BP = 90-95° C./ |
| | 1 mm Hg |
| 3.19 g | Deionized water |

The apparatus, test procedure, synthesis, swelling tests, etc. were analogous to P-054. Initiator solution was the same as for P-017.

### Synthesis of the Crosslinking Monomer:

The difference in reactivities of the two isocyanate groups of isophorone diisocyanate was utilized in the first reaction step by reacting with N-(tert-butylamino)ethyl methacrylate. The maximum reaction temperature was held at 30° C. to maintain selectivity (at higher temperatures, the reactivity of the other NCO group increases). In a second reaction step, the remaining isocyanate group was blocked using ethyl methyl ketoxime (EMKO) in order to avoid premature reaction with the aqueous bath during drying while crosslinking was being carried out in the clamping frame. Thus, only during condensation is the protected isocyanate unblocked and released for the crosslinking reaction. Use of a catalyst may be dispensed with in this synthesis, since amines (N-(tert-butylamino)ethyl methacrylate) are approximately 4000 times more nucleophilic than OH groups (see step 2 in the p-EMKO-TDI-o-HEMA synthesis). This generally increases the storage stability of the subsequent aqueous polymer dispersion, since trace amounts of catalyst may be avoided. The reaction also takes place in the substance, i.e., in the absence of solvent, which additionally simplifies the overall synthesis and workup. Literature reference: Degussa AG—Coatings & Colorants, VESTANAT IPDI—Properties & Handling, product information sheet 43.01.062d/02.06/500/jd/g3, 2009, 1-16; Knebel, J., Breiner, C., Schmitt, B., “Novel polymerizable isocyanate and polymers containing said isocyanate”, WO 2009/024493 A2.

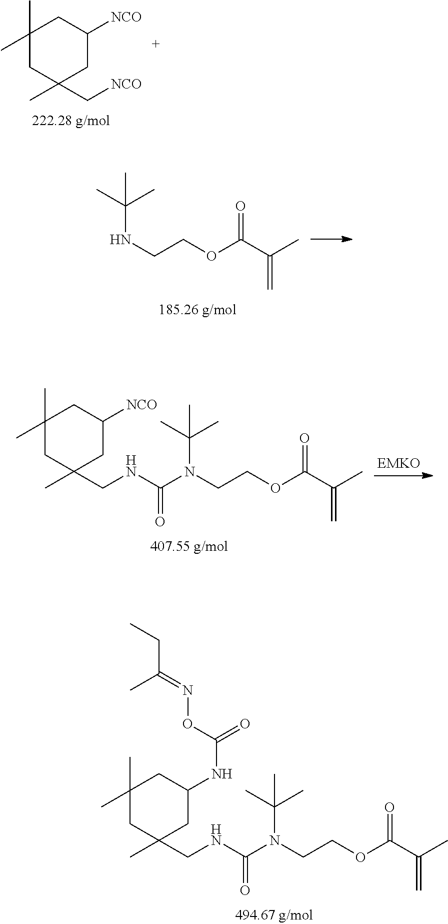

### Summary:

The radical polymerization for the crosslinking agent screening, using the EMKO alkenyl isocyanate crosslinking monomer, proceeded successfully. It was possible to synthesize a polymer having very good swelling capability within a very short time.

### Polymer Compound P-059

mPEG methacrylates have an optimized balance between water absorption and elongation at break for n=3 ethylene oxide units. Compound P-059 was prepared analogously to P-042, using ethyl triglycol methacrylate (ETMA) instead of mPEG 350 methacrylate (n=8) as hydrophilic monomer.

Ethyl triglycol methacrylate (ETMA) has the advantage over methyl triglycol methacrylate that it is commercially available in large quantities. Literature reference: Kumakura, M., Kaetsu, I., Physical characterization and molecular structure of hydrophilic polymers obtained by radiation cast-polymerization of methoxypolyethylene glycol methacrylate monomers for biomedical applications, Journal of Materials Science (18), 1983, 2430-2436.

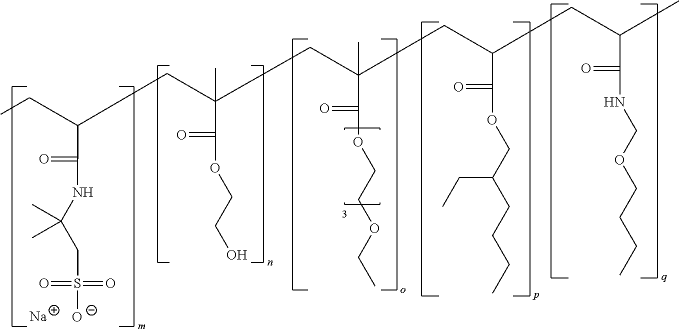

### Starting Product Solution:

| Weigh-in | Product description |
|---|---|
| 4.96 g | AMPS Na 2403 monomer: 2-acrylamido-2-methylpropane |
| | sodium sulfonate |
| 2.48 g | >97% Visiomer ETMA: ethyl triglycol methacrylate (ETMA), |
| | Evonik Industries, M = 246.30 g/mol, d = 1.02 |
| | g/mL, BP = 292° C. |
| 0.83 g | 97% 2-hydroxyethyl methacrylate (HEMA), stabilized, |
| | Acros Organics |
| 1.65 g | 99% 2-ethylhexyl acrylate, stabilized, Acros Organics |
| 0.83 g | Cylink NBMA monomer: >82% N-(butoxymethyl)acrylamide, |
| | Cytec Industries |
| 5.0 g | 2-Propanol, Schweizerhall, M = 60.10 g/mol, d = 0.785 |
| | g/mL, BP = 82° C. |
| 10.0 g | Dipropylene glycol, d = 1.023 g/mL, BP = 90-95° C./ |
| | 1 mm Hg |
| 4.5 g | Deionized water |

The apparatus, test procedure, synthesis, swelling tests, etc. were analogous to P-054. Initiator solution was the same as for P-017. Swelling behavior: Water was poured over slightly dried polymer, causing the polymer to swell within minutes and become detached. The permanence was poor, with the granular (brittle) polymer falling apart.

### Summary:

The radical polymerization using ethyl triglycol methacrylate (ETMA) instead of mPEG 350 methacrylate as hydrophilic monomer proceeded successfully. To simplify the subsequent finishing tests, the reaction solution was diluted with water to a polymer concentration of 12.0%. It was possible to synthesize a polymer having very good swelling capability within a very short time.

### Polymer Compound P-060

Based on the composition of P-042, a test was conducted to replace the sodium counterions of AMPS with lithium ions. This was based on the presumption that the sodium counterions increase the crystallinity and the brittleness of the polymers, thus making the polymers less elastic. For this purpose, the AMPS sodium salt was replaced with pure AMPS, which was neutralized with lithium hydroxide. The aim was to investigate whether the brittleness of the polymers during swelling with water decreased as the result of the smaller and more mobile lithium counterions, with the aim of positively influencing the wash permanence. The polymerization was carried out undiluted.

When carrying out the AMPS neutralizations, in general it is important to note that the neutralizations must take place with cooling (<10° C.), since AMPS tends to undergo autopolymerization at elevated temperatures, and bases are able to initiate Michael addition at the acrylate monomers. For the same reasons, a pH range of 7.0-7.5 must also be maintained, since at pH<7.0 the autopolymerization is favored, and at pH>7.5 the Michael addition is favored.

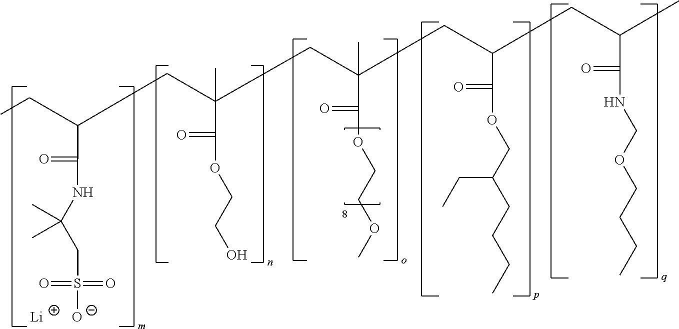

### AMPS Lithium Solution:

| Weigh-in | Product description |
|---|---|
| 4.52 g, | >99.0% AMPS 2401 monomer: 2-acrylamido-2-methyl- |
| 21.81 mmol | propanesulfonic acid, Lubrizol, M = 207.25 g/mol |
| 0.92 g | ≧99% lithium hydroxide monohydrate, Fluka |
| 4.56 g | Deionized water |

3 g water was provided. The AMPS was then added, with stirring, and neutralized with the lithium hydroxide monohydrate. To avoid autopolymerization and Michael addition, the strongly exothermic neutralization reaction was held at a maximum reaction temperature of 10° C., using a cooling bath. Lastly, the solution was diluted with the remaining water, resulting in 10 g of solution, pH=7.

### Starting Product Solution:

| Weigh-in | Product description |
|---|---|
| 4.96 g | AMPS lithium solution (equimolar quantity relative to |
| | AMPS from P-042) |
| 2.48 g | >95% mPEG 350 methacrylate, Evonik Industries |
| 0.83 g | 97% 2-hydroxyethyl methacrylate (HEMA), stabilized, |
| | Acros Organics |
| 1.65 g | 99% 2-ethylhexyl acrylate, stabilized, Acros Organics |
| 0.83 g | Cylink NBMA monomer: >82% N-(butoxymethyl)acrylamide, |
| | Cytec Industries |
| 5.0 g | 2-Propanol, Schweizerhall, M = 60.10 g/mol, d = 0.785 g/mL, |
| | BP = 82° C. |
| 10.0 g | Dipropylene glycol, d = 1.023 g/mL, BP = 90-95° C./ |
| | 1 mm Hg |
| 4.5 g | Deionized water |

The apparatus, test procedure, synthesis, swelling tests, etc. were analogous to P-054. Initiator solution was the same as for P-017. Swelling behavior: Water was poured over slightly dried polymer, causing the polymer to swell within minutes and become detached. The polymer disintegrated into small pieces, and the permanence appeared to be poor.

### Summary:

The radical polymerization using AMPS lithium instead of AMPS sodium proceeded successfully. To simplify the subsequent finishing tests, the reaction solution was diluted with water to a polymer concentration of 12.0%. It was possible to synthesize a polymer having very good swelling capability within a very short time.

### Polymer Compound P-061

Based on the composition of P-042, a test was conducted to replace the sodium counterions of AMPS with ammonium ions. For this purpose, pure AMPS was neutralized with ammonium hydroxide in order to produce ammonium counterions.

### AMPS Ammonium Solution:

| Weigh-in | Product description |
|---|---|
| 4.52 g, | AMPS 2401 monomer: >99.0% 2-acrylamido-2-methyl- |
| 21.81 mmol | propanesulfonic acid, Lubrizol, M = 207.25 g/mol |
| 1.49 g | 25% ammonium hydroxide solution, NH₃ in water, Fluka |
| 3.99 g | Deionized water |

3 g water was provided. The AMPS was then added, with stirring, and neutralized with the lithium ammonium solution. To avoid autopolymerization and Michael addition, the strongly exothermic neutralization reaction was held at a maximum reaction temperature of 10° C., using a cooling bath. Lastly, the solution was diluted with the remaining water, resulting in 10 g of solution, pH=7.

The starting product solution was the same as for P-059, using 4.96 g AMPS ammonium solution instead of the AMPS lithium solution. The apparatus, test procedure, synthesis, swelling tests, etc. were analogous to P-054. Initiator solution was the same as for P-017. Swelling behavior: Water was poured over slightly dried polymer, causing the polymer to swell within minutes and become detached. Although the polymer fell apart into rough pieces, the permanence appeared to be poor.

### Summary:

The radical polymerization using AMPS ammonium instead of AMPS sodium proceeded successfully. After a reaction time of 4 h, the conversion was 99.5%. To simplify the subsequent finishing tests, the reaction solution was diluted with water to a polymer concentration of 12.0%. It was possible to synthesize a polymer having very good swelling capability within a very short time.

### Polymer Compound P-062

Based on the composition of P-042, a test was conducted to replace the sodium counterions of AMPS with triethylammonium ions. For this purpose, pure AMPS was neutralized with triethylamine in order to investigate the influence of the relatively voluminous ethyl groups of triethylammonium compared to the lithium, sodium, and ammonium counterions.

### AMPS Triethylammonium Solution:

| Weigh-in | Product description |
|---|---|
| 4.52 g, | AMPS 2401 monomer: >99.0% 2-acrylamido-2-methyl- |
| 21.81 mmol | propanesulfonic acid, Lubrizol, M = 207.25 g/mol |
| 2.21 g | ≧98% triethylamine, Fluka |
| 3.27 g | Deionized water |

3 g water was provided. The AMPS was then added, with stirring, and neutralized with triethylamine. To avoid autopolymerization and Michael addition, the strongly exothermic neutralization reaction was held at a maximum reaction temperature of 70° C., using a cooling bath. Lastly, the solution was diluted with the remaining water, resulting in 10 g of solution, pH=7.

The starting product solution was the same as for P-059, using 4.96 g AMPS triethylammonium solution instead of the AMPS lithium solution. The apparatus, test procedure, synthesis, swelling tests, etc. were analogous to P-054. Initiator solution was the same as for P-017. Swelling behavior: Water was poured over slightly dried polymer, causing the polymer to swell within minutes and become detached. Although the polymer fell apart into rough pieces, the permanence appeared to be poor.

### Summary:

The radical polymerization using AMPS triethylammonium instead of AMPS sodium proceeded successfully. After a reaction time of 4 h, the conversion was 93.0%. To simplify the subsequent finishing tests, the reaction solution was diluted with water to a polymer concentration of 12.0%. It was possible to synthesize a polymer having very good swelling capability within a very short time.

### Polymer Compound P-063

Based on the composition of P-042, a test was conducted to replace the sodium counterions of AMPS with 1-methylimidazolium ions. For this purpose, pure AMPS was neutralized with 1-methylimidazole in order to produce 1-methylimidazolium counterions.

### AMPS 1-Methylimidazolium Solution:

| Weigh-in | Product description |
|---|---|
| 4.52 g, | AMPS 2401 monomer: >99.0% 2-acrylamido-2-methyl- |
| 21.81 mmol | propanesulfonic acid, Lubrizol, M = 207.25 g/mol |
| 1.79 g | ≧99% 1-methylimidazole, Fluka |
| 3.69 g | Deionized water |

3 g water was provided. The AMPS was then added. with stirring, and neutralized with 1-methylimidazolium. To avoid autopolymerization and Michael addition, the strongly exothermic neutralization reaction was held at a maximum reaction temperature of 10° C., using a cooling bath. Lastly, the solution was diluted with the remaining water, resulting in 10 g of solution, pH=7.

The starting product solution was the same as for P-059, using 4.96 g AMPS 1-methylimidazolium solution instead of the AMPS lithium solution. The apparatus, test procedure, synthesis, swelling tests, etc. were analogous to P-054. Initiator solution was the same as for P-017. Swelling behavior: Water was poured over slightly dried polymer, causing the polymer to swell within minutes and become detached. Although the polymer fell apart into rough pieces, the permanence appeared to be poor.

### Summary:

The radical polymerization using AMPS 1-methylimidazolium instead of AMPS sodium proceeded successfully. After a reaction time of 4 h, the conversion was 93.2%. To simplify the subsequent finishing tests, the reaction solution was diluted with water to a polymer concentration of 12.0%. It was possible to synthesize a polymer having very good swelling capability within a very short time.

### Polymer Compound P-064

Based on the composition of P-042, a test was conducted to replace the Na⁺ counterions of AMPS with 4-methylmorpholinium ions. For this purpose, pure AMPS was neutralized with 4-methylmorpholine in order to produce 1-methylimidazolium counterions.

### AMPS 4-Methylmorpholinium Solution:

| Weigh-in | Product description |
|---|---|
| 4.52 g, | AMPS 2401 monomer: >99.0% 2-acrylamido-2-methyl- |
| 21.81 mmol | propanesulfonic acid, Lubrizol, M = 207.25 g/mol |
| 1.79 g | 99% 4-methylmorpholine, Acros Organics |
| 3.27 g | Deionized water |

3 g water was provided. The AMPS was then added, with stirring, and neutralized with 4-methylmorpholine. To avoid autopolymerization and Michael addition, the strongly exothermic neutralization reaction was held at a maximum reaction temperature of 10° C., using a cooling bath. Lastly, the solution was diluted with the remaining water, resulting in 10 g of solution, pH=7.

The starting product solution was the same as for P-059, using 4.96 g AMPS 4-methylmorpholine solution instead of the AMPS lithium solution. The apparatus, test procedure, synthesis, swelling tests, etc. were analogous to P-054. Initiator solution was the same as for P-017. Swelling behavior: Water was poured over slightly dried polymer, causing the polymer to swell within minutes and become detached. Although the polymer fell apart into rough pieces, the permanence appeared to be poor.

### Summary:

The radical polymerization using AMPS 4-methylimidazolium instead of AMPS sodium proceeded successfully. After a reaction time of 4 h. the conversion was 92.7%. To simplify the subsequent finishing tests, the reaction solution was diluted with water to a polymer concentration of 12.0%. It was possible to synthesize a polymer having very good swelling capability within a very short time.

### Polymer Compound P-065

Based on the composition of P-054, the polymerization was repeated using 1% N,N′-methylene-bis-acrylamide (MBAm) instead of the NBMA crosslinking agent. The aim was to investigate whether the brittleness of the polymers decreased when swelled with water, in order to positively influence the wash permanence. The polymerization was carried out undiluted.

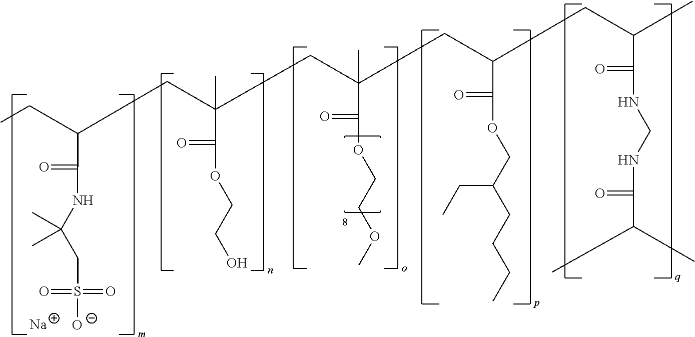

### Starting Product Solution:

| Weigh-in | | Product description |
|---|---|---|
| 4.96 | g | AMPS Na 2403 monomer: 2-acrylamido-2-methylpropane |
| | | sodium sulfonate |
| 2.48 | g | >95% mPEG 350 methacrylate, Evonik Industries |
| 0.83 | g | 97% 2-hydroxyethyl methacrylate (HEMA), stabilized, |
| | | Acros Organics |
| 2.4 | g | 99% 2-ethylhexyl acrylate, stabilized, Acros Organics |
| 0.08 | g | ≧99% N,N′-methylene-bis-acrylamide (MBAm), Fluka, |
| | | M = 154.17 g/mol |
| 5.0 | g | 2-Propanol, Schweizerhall, M = 60.10 g/mol, d = 0.785 |
| | | g/mL, BP = 82° C. |
| 10.0 | g | Dipropylene glycol, d = 1.023 g/mL, BP = 90-95° C./ |
| | | 1 mm Hg |
| 4.5 | g | Deionized water |

The apparatus, test procedure, synthesis, swelling tests, etc. were analogous to P-054. Swelling behavior: Water was poured over slightly dried polymer, causing the polymer to swell with film formation within minutes, with hardly any detachment. The polymer did not fall apart into rough pieces, and according to visual observation appeared to have fairly good crosslinking.

### Summary:

The radical polymerization using 1% N,N′-methylene-bis-acrylamide (MBAm) proceeded successfully. However, due to the biofunctionalized N,N′-methylene-bis-acrylamide, the viscosity of the reaction solution increased markedly on account of the network formation during the polymerization process. To simplify the subsequent finishing tests, the reaction solution was diluted with water to a polymer concentration of 12.0%. It was possible to synthesize a polymer having very good swelling capability within a very short time.

### Polymer Compound P-066

Based on the composition of P-042, the polymerization was repeated using 9% NBMA crosslinking agent and 1% N,N′-methylene-bis-acrylamide (MBAm). The aim was to investigate whether the brittleness of the polymers decreased when swelled with water, in order to positively influence the wash permanence. The polymerization was carried out undiluted.

### Starting Product Solution:

| Weigh-in | | Product description |
|---|---|---|
| 4.96 | g | AMPS Na 2403 monomer: 2-acrylamido-2-methylpropane |
| | | sodium sulfonate |
| 2.48 | g | >95% mPEG 350 methacrylate, Evonik Industries |
| 0.83 | g | 97% 2-hydroxyethyl methacrylate (HEMA), stabilized, |
| | | Acros Organics |
| 1.65 | g | 99% 2-ethylhexyl acrylate, stabilized, Acros Organics |
| 0.74 | g | Cylink NBMA monomer: >82% N-(butoxymethyl)acrylamide, |
| | | Cytec Industries |
| 0.08 | g | ≧99% N,N′-methylene-bis-acrylamide (MBAm), Fluka, |
| | | M = 154.17 g/mol |
| 5.0 | g | 2-Propanol, Schweizerhall, M = 60.10 g/mol, d = 0.785 |
| | | g/mL, BP = 82° C. |
| 10.0 | g | Dipropylene glycol, d = 1.023 g/mL, BP = 90-95° C./ |
| | | 1 mm Hg |
| 4.5 | g | Deionized water |

The apparatus, test procedure, synthesis, swelling tests, etc. were analogous to P-054. Swelling behavior: Water was poured over slightly dried polymer, causing the polymer to swell within minutes and become detached. The polymer disintegrated into fine pieces, and the permanence appeared to be very poor.

### Summary:

The radical polymerization using 9% N-(butoxymethyl)acrylamide and 1% N,N′-methylene-bis-acrylamide (MBAm) as crosslinking monomers proceeded successfully. However, due to the biofunctionalized N,N′-methylene-bis-acrylamide, the viscosity of the reaction solution increased Markedly on account of the network formation during the polymerization process, resulting in a gel-like solution. To simplify the subsequent finishing tests, the reaction solution was diluted with water to a polymer concentration of 12.0%. It was possible to synthesize a polymer having very good swelling capability within a very short time.

### P-042 Scale-Up No. 1

The polymerization was carried out as 30% radical emulsion polymerization, not as 12% solution polymerization as in test P-042, in a 1-L glass apparatus at a reaction temperature of 70° C., using V-50 as initiator. In addition, Disponil AFX and sodium dodecylbenzenesulfonate were added as further emulsifiers. The aim was to investigate whether P-042 could be easily prepared as a 0.5 kg scale-up by emulsion polymerization.

### Starting Product Solution:

| Weigh-in | | Product description |
|---|---|---|
| 90.0 | g | AMPS Na 2403 monomer: 2-acrylamido-2-methylpropane |
| | | sodium sulfonate |
| 45.0 | g | >95% mPEG 350 methacrylate, Evonik Industries |
| 15.0 | g | 97% 2-hydroxyethyl methacrylate (HEMA), stabilized, |
| | | Acros Organics |
| 30.0 | g | 99% 2-ethylhexyl acrylate, stabilized, Acros Organics |
| 15.0 | g | Cylink NBMA monomer: 81% N-(butoxymethyl)acrylamide, |
| | | Cytec Industries |
| 4.5 | g | 80% Disponil AFX 1080, Cognis |
| 3.0 | g | Sodium dodecyl sulfate (SDS), p.a., Serva Feinbiochemika, |
| | | M = 288.38 g/mol |
| 5.0 | g | 30% sodium dodecylbenzenesulfonate solution in water, |
| | | M = 348.48 g/mol |
| 25.0 | g | Dipropylene glycol, d = 1.023 g/mL, BP = 90-95° C./ |
| | | 1 mm Hg |
| 222.8 | g | Deionized water |

Initiator solution was the same as for P-017. Prehomogenization: The starting product solution (pH=5.7) was passed through a high-pressure homogenizer five times at 600 bar, resulting in an emulsion. Apparatus: 1-L four-neck round bottom flask with stirrer, reflux condenser, septum. and temperature sensor. A discharge line with a stopcock on the reflux condenser was used for evacuation and flushing with nitrogen.

Synthesis: 455 g homogenized starting product solution was placed in the apparatus and heated to 70° C., using a heating mantle. When the temperature reached approximately 60° C., the apparatus was evacuated and flushed with nitrogen three times each to remove atmospheric oxygen (inhibitor). The apparatus was back-flushed with nitrogen to ensure pressure compensation over the entire polymerization. 25 g of initiator solution was then metered through the septum via a syringe, with good stirring. Only slightly exothermic conditions were detectable. The reaction temperature was held constant at 70° C. After approximately 10 min, the reaction mixture became highly viscous and then abruptly polymerized to completion. All of the water was absorbed into the batch of polymer that formed, leaving a solid, gel-like polymer block in the reactor. The 30% radical emulsion polymerization was too concentrated.

### P-042 Scale-Up No. 2

The synthesis was carried out analogously to P-042 scale-up No. 1, but as 15% radical emulsion polymerization. The initiator solution was used at twice the concentration (the same quantity of initiator solution with half the quantity of monomers) in order to maintain the reaction rate without large losses.

### Starting Product Solution:

| Weigh-in | | Product description |
|---|---|---|
| 45.0 | g | AMPS Na 2403 monomer: 2-acrylamido-2-methylpropane |
| | | sodium sulfonate |
| 22.5 | g | >95% mPEG 350 methacrylate, Evonik Industries |
| 7.5 | g | 97% 2-hydroxyethyl methacrylate (HEMA), stabilized, |
| | | Acros Organics |
| 15.0 | g | 99% 2-ethylhexyl acrylate, stabilized, Acros Organics |
| 7.5 | g | Cylink NBMA monomer: 81% N-(butoxymethyl)acrylamide, |
| | | Cytec Industries |
| 4.5 | g | 80% Disponil AFX 1080, Cognis |
| 3.0 | g | Sodium dodecyl sulfate (SDS), p.a., Serva Feinbiochemika, |
| | | M = 288.38 g/mol |
| 5.0 | g | 30% sodium dodecylbenzenesulfonate solution in water, |
| | | M = 348.48 g/mol |
| 25.0 | g | Dipropylene glycol, d = 1.023 g/mL, BP = 90-95° C./ |
| | | 1 mm Hg |
| 222.8 | g | Deionized water |

Initiator solution was the same as for P-017. Prehomogenization and apparatus were the same as for P-042 scale-up No. 1.

Synthesis: 455 g homogenized starting product solution was placed in the apparatus and heated to 70° C., using a heating mantle. When the temperature reached approximately 60° C., the apparatus was evacuated and flushed with nitrogen three times each to remove atmospheric oxygen (inhibitor). The apparatus was back-flushed with nitrogen to ensure pressure compensation over the entire polymerization. 25 g of initiator solution was then metered through the septum via a syringe, with good stirring. Only slightly exothermic conditions were detectable. The reaction temperature was held constant at 70° C. After 30 min the remaining 27 g initiator solution was added via syringe (no subsequent exothermic conditions were detectable). An emulsion with a slight reddish sheen was obtained, on which a foam layer approximately 1-2 cm thick was present during the entire reaction. Swelling behavior: Water was poured over slightly dried polymer, causing the polymer to swell to several times its original volume within minutes and become detached. Permanence appeared to be poor.

### Summary:

The 15% radical emulsion polymerization as a 0.5 kg scale-up proceeded successfully, although the conversion was only 91.7% after a reaction time of 4 h. The lack of coagulate formation on the reaction flask was probably due to loss of starting product solution during homogenization (dead volume). Determination of the particle size distribution by photon correlation spectroscopy (PCS) showed a multidisperse emulsion droplet distribution having hydrodynamic droplet diameters of 100 nm (3.9 vol-%), 1094 nm (92.2 vol-%), and 4602 nm (3.9 vol-%). To simplify the subsequent finishing tests, the reaction solution was diluted with water to a polymer concentration of 12.0%. It was possible to synthesize a polymer having very good swelling capability within a very short time.

### P-044 Scale-Up No. 1

Scale-up of P-044 to 0.5 kg for preparing poly(2-acrylamido-2-methylpropane sodium sulfonate-stat-2-ethylhexyl acrylate-stat-mPEG 350 methacrylate-stat-N-(butoxymethyl)acrylamide).

Because the previous emulsion system is not particularly stable, alternatives were sought. The starting product solution was a versatile monomer system having hydrophilic and hydrophobic monomers as well as nonionic and ionic monomers, which previously has resulted in phase separation after approximately 10 minutes in the unstirred system. This may cause problems for polymerization in a large-scale reactor, since the monomer phase floating on top could undergo bulk polymerization. For this reason, preliminary tests were conducted using the following nonionic emulsifiers: Marlipal 013/30, Marlipal 013/50, Mulsifan RTI10, Hostapur OS Liquid, Marlosol OL7, and Marlowet R 40. The most stable emulsion was obtained using 3-5% Marlowet R 40. It was shown that addition of sodium dodecyl sulfate (SDS) and sodium dodecylbenzenesulfonate once again resulted in phase separation. Therefore, Marlowet R 40 was used as the sole emulsifier. However, this material forms a stable emulsion only when the monomers are placed in the apparatus first, Marlowet R 40 is. added, and only then water is added at the end. In contrast. an unstable emulsion results when the water is added first, and then the emulsifier.

The polymerization was based on P-042 scale-up No. 2, except that only 1% V-50 was used as initiator (instead of 2% V-50, as for P-042 scale-up No. 2), and the polymerization was carried out at a reaction temperature of 70° C. Monomer AMPS Na 2405 was used for the production instead of AMPS Na 2403. AMPS Na 2405 is authorized for foods as well as for application to the skin, since the acrylamide and acrylonitrile content is less than 0.05%. In addition, the polymerization was carried out without prehomogenization, and the formulation of P-044 (without HEMA) was used as the basis since it has shown the best results in the finishing tests. The aim was to investigate whether P-044 could be easily prepared as a 0.5 kg scale-up by emulsion polymerization.

### Starting Product Solution:

| Weigh-in | Product description |
|---|---|
| 45.0 g | AMPS Na 2405 monomer: 2-acrylamido-2-methylpropane |
| | sodium sulfonate (contains approx. 50% water), |
| | Lubrizol, M = 229.23 g/mol, d = 1.21 g/mL |
| 30.0 g | >95% mPEG 350 methacrylate, Evonik Industries |
| 15.0 g | 99% 2-ethylhexyl acrylate, stabilized, Acros Organics |
| 7.5 g | Cylink NBMA monomer: 81% N-(butoxymethyl)acrylamide, |
| | Cytec Industries |
| 2.3 g | Marlowet R 40: 83% PEG-40 castor oil, Sasol, |
| | M = 2695 g/mol, d = 1.06 g/mL, MP = 17° C. |
| 15.0 g | Dipropylene glycol, d = 1.023 g/mL, BP = 90-95° C./ |
| | 1 mm Hg |
| 375.2 g | Deionized water |

Initiator solution was the same as for P-017. Prehomogenization and apparatus were the same as for P-042 scale-up No. 1.

Synthesis: 97.5 g monomers, 2.3 g Marlowet R40, and 15.0 g dipropylene glycol were placed in the apparatus. 375.2 g water was then added slowly dropwise, with intensive stirring, thus emulsifying the system (white emulsion). The monomer emulsion was heated to 70° C. using a heating mantle, with stirring. When the temperature reached approximately 60° C., the apparatus was evacuated and flushed with nitrogen three times each to remove atmospheric oxygen (inhibitor). The apparatus was back-flushed with nitrogen to ensure pressure compensation over the entire polymerization. 5 g initiator solution was then metered through the septum via a syringe, with good stirring. Only slightly exothermic conditions were detectable. The reaction temperature was held constant at 70° C. After 30 min the remaining 5 g initiator solution was added via syringe (no subsequent exothermic conditions were detectable), resulting in a white, viscous emulsion. Swelling behavior: Water was poured over slightly dried polymer, causing the polymer to swell to several times its original volume within minutes and become detached. The permanence appeared to be poor.

Summary: The 15% radical emulsion polymerization of P-044 as a 0.5 kg scale-up proceeded successfully. The conversion was 98.0% after a reaction time of 4 h, using 1% V-50 as initiator. Despite a new emulsifier system and the lack of prehomogenization, once again no coagulate formed on the reaction flask. Determination of the particle size distribution by photon correlation spectroscopy (PCS) showed a monodisperse emulsion droplet size having a hydrodynamic diameter of 1265 nm. It was possible to synthesize a polymer having very good swelling capability within a very short time.

### P-067 Scale-Up

Based on the scale-up of P-044, the monomer fractions used were reversed, with 40% mPEG 350 methacrylate and 20% 2-ethylhexyl acrylate. Polymer P-067 thus had the following base composition: 30 wt.-% AMPS sodium salt, 20 wt.-% mPEG 350 methacrylate, 40 wt.-% 2-ethylhexyl acrylate, 10 wt.-% NBMA crosslinking agent.

Thus, in desorption tests it was possible to investigate the desorption rate of the active substances as a function of the amphiphilic character of the polymer donor layer. If the active substances are delivered to the skin too quickly, the donor layer must have a more lipophilic design, and conversely, if the active substance desorption is too low the donor layer must have a more hydrophilic design.

### Starting Product Solution:

| Weigh-in | | Product description |
|---|---|---|
| 45.0 | g | AMPS Na 2405 monomer: 2-acrylamido-2-methylpropane |
| | | sodium sulfonate |
| 15.0 | g | >95% mPEG 350 methacrylate, Evonik Industries |
| 30.0 | g | 99% 2-ethylhexyl acrylate, stabilized, Acros Organics |
| 7.5 | g | Cylink NBMA monomer: 81% N-(butoxymethyl)acrylamide, |
| | | Cytec Industries |
| 2.3 | g | Marlowet R 40: 83% PEG-40 castor oil, Sasol |
| 15.0 | g | Dipropylene glycol, d = 1.023 g/mL, BP = 90-95° C./ |
| | | 1 mm Hg |
| 375.2 | g | Deionized water |

Initiator solution, prehomogenization, apparatus, and synthesis were the same as for P-044 scale-up No. 1. Swelling behavior: Water was poured over slightly dried polymer, causing the polymer to swell to several times its original volume within minutes and become only slightly detached. The permanence appeared to be good.

### P-044 Scale-Up No. 2

Scale-up of P-044 to 4.5 kg for preparing poly(2-acrylamido-2-methylpropane sodium sulfonate-stat-2-ethylhexyl acrylate-stat-mPEG 350 methacrylate-stat-N-(butoxymethyl)acrylamide).

The polymerization was carried out in a 6-L four-neck round bottom flask at a reaction temperature of 70° C.; using V-50 as initiator. Since coarse particles were visually detectable in the diluted sample in the emulsion polymerization of the P-067 scale-up without prehomogenization, the present polymerization was prehomogenized. To save time, however, the starting product solution was passed through the high-pressure homogenizer only three times at 600 bar. The aim was to investigate whether P-044 could be easily prepared as a 4.5 kg scale-up by emulsion polymerization.

### Starting Product Solution:

| Weigh-in | Product description |
|---|---|
| 405.0 g | AMPS Na 2405 monomer: 2-acrylamido-2-methylpropane |
| | sodium sulfonate |
| 270.0 g | >95% mPEG 350 methacrylate, Evonik Industries |
| 135.0 g | 99% 2-ethylhexyl acrylate, stabilized, Acros Organics |
| 67.5 g | Cylink NBMA monomer: 81% N-(butoxymethyl)acrylamide, |
| | Cytec Industries |
| 20.5 g | Marlowet R 40: 83% PEG-40 castor oil, Sasol |
| 135.0 g | Dipropylene glycol, d = 1.023 g/mL, BP = 90-95° C./ |
| | 1 mm Hg |
| 3367.0 g | Deionized water |

Initiator solution was the same as for P-017. Apparatus: 6-L four-neck round bottom flask with stirrer, reflux condenser, 250-mL feed funnel with pressure compensation, and temperature sensor. A discharge line with a stopcock on the reflux condenser was used for evacuation and flushing with nitrogen. Prehomogenization: The starting product solution (pH=6.1) was passed through the high-pressure homogenizer three times at 600 bar, resulting in an emulsion. 1 g homogenized starting product solution was diluted 1:20 with 19 g water. Viscosity: 0.98 mPa·s. Particle size distribution (PCS measurement): peak 1: d(H)=1224 nm−77.0 vol-%; peak 2: d(H)=95 nm−23.0 vol-%; peak 3: d(H): No signal was present.

Synthesis: 4400 g homogenized starting product solution was placed in the apparatus and heated to 70° C., using a heating mantle. 100 g initiator solution was then filled into the feed funnel on the apparatus. When the temperature reached approximately 60° C., the apparatus was evacuated and flushed with nitrogen three times each to remove atmospheric oxygen (inhibitor). The apparatus was back-flushed with nitrogen to ensure pressure compensation over the entire polymerization. 50 g initiator solution was then metered through the feed funnel, with good stirring. Exothermic conditions were detectable which caused the reaction solution to heat up to 80° C. For this reason, the temperature of the heating mantle was temporarily lowered to ensure better cooling. The reaction temperature was held constant at 70° C. After 30 min the remaining 50 g initiator solution was added (no subsequent exothermic conditions were detectable), resulting in a white, viscous emulsion on which a foam layer was present. No coagulate was formed. Swelling behavior: Water was poured over slightly dried polymer, causing the polymer to swell to several times its original volume within minutes and become detached. The permanence appeared to be poor.

Summary: The 15% radical emulsion polymerization of P-044 as a 4.5 kg scale-up proceeded successfully. The conversion was 97.6% after a reaction time of 4 h. Once again, no coagulate formed on the reaction flask. Determination of the particle size distribution by photon correlation spectroscopy (PCS) showed a multidisperse emulsion droplet distribution having hydrodynamic droplet diameters of 1126 nm (92.3 vol-%), 202 nm (4.9 vol-%), and 5472 nm (2.8 vol-%), although this measuring result was not very accurate. The reason could be that the starting product solution was passed only three times, instead of five times as previously, through the high-pressure homogenizer at 600 bar. It was possible to synthesize a polymer having very good swelling capability within a very short time.

The examples of polymer compounds according to the invention discussed in this section in conjunction with a suitable binder form wash-permanent finishes on textiles. Corresponding methods are described in the next section. It has been found that P-044 and P-067 are able to form textile finishes that withstand 100 washes at 60° C. for 50-55 min without significant impairment of the donor layer.

### B. Finishing of Textile Surfaces

The permanence of the finishing of fabrics using the polymers according to the invention and the effective available surface charge of the finishing layers were investigated.

### Test Series 1

Finishing of the Fabric:

A fabric made of 100% polyamide (PA) was used for each of the finishing tests (prefixed charmeuse, weight per unit area 135 g/m²; Fussenegger Textilveredelung GmbH, AT-6850 Dornbirn; Tricot manufacturer: Huber Tricot GmbH, AT-6841 Mäder; Prod. No. 11065). The bath batches were 200 g polymer/kg aqueous baths, or optionally, a 100 g crosslinking catalyst stock solution/kg bath. The mentioned catalyst stock solution was composed of 50 g MgCl₂×6 H₂O/kg stock solution and 20 g L-(+) tartaric acid/kg stock solution. The finishing was carried out by padding the PA fabric with the polymer dispersion bath (roller pressure 15 bar, fabric speed 2 m/min), subsequently drying (circulation air temperature 100° C., 3 min), and condensing/fixing the finish (circulation air temperature 150° C., 5 min).

Permanence of the Finish:

The permanence of the finish was determined by Soxhlet extraction. For this purpose, in each case two samples of finished textile fabric, 12.5 g each, were extracted with methanol over a period of 3 h. The results of the different finishing variants tested are as follows:

TABLE
Permanence of the finish

| | | pH of the | | Coating quantity of | Remaining coating |
|---|---|---|---|---|---|
| | | polymer | Crosslinking | finish (wt.-% | quantity of finish |
| Finish | Polymer | dispersion | catalyst used | fabric sample) | after extraction |
| A-001 | P-002 | 3.3 | No | 3.35% | 89% |
| A-002 | P-002 | 3.3 | Yes | 3.46% | 90% |
| A-003 | P-004 | 8.5 | No | 3.73% | 68% |
| A-004 | P-004 | 8.5 | Yes | 3.76% | 77% |
| A-007 | P-005 | approx. 7 | Yes | 4.44% | 7% |
| A-008 | P-008 | approx. 7 | Yes | 1.00% | 0% |
| A-009 | P-009 | approx. 7 | Yes | 1.76% | 24% |

### Surface Charge of the Finishing Layer

The surface charge of the finished fabric samples was determined by charge titration, using a charge analyzing system (CAS) (AFG Analytic GmbH, Leipzig, DE; Model No. B390/B422/B490). Sample preparation: 0.5 g of fabric sample was ground, using a 20-mm tungsten carbide grinding ball in a screw-on 25-mL grinding cup made of hardened specialty steel, at −196° C. (liquid nitrogen) for 2×2 min at 30 Hz (Retsch MM400 ball mill).

To determine the surface charge, 4.8 g water was placed in the PTFE measuring cell of the CAS, and 0.2 g of the ground fabric sample was added and mixed. The PTFE measuring cell was suspended in an ultrasonic bath for 2 min. The measuring flask was then inserted into the measuring cell, and the charge titration was carried out with cationic polyelectrolyte solution, 0.001N poly(diallyldimethylammonium chloride) (polyDADMAC).

TABLE
Results of charge titration

| | | | Surface charge [μmol | | |
|---|---|---|---|---|---|
| | | Titration | negative charge/g sample] **) | | |
| | Polymer, catalyst, | solution | | Due to | |
| Finish No. | storage | consumed [mL] | Total | finishing layer *) | Theoretical |
| A-001 | P-002 without catalyst | 0.30 | 1.50 | 0.87 | 86.02 |
| A-001 | P-002 without catalyst, | 0.59 | 2.95 | 2.32 | 86.02 |
| | 4 weeks storage | | | | |
| A-002 | P-002 with catalyst | 0.71 | 3.55 | 2.92 | 88.93 |
| A-002 | P-002 with catalyst, | 0.65 | 3.25 | 2.62 | 88.93 |
| | 4 weeks storage | | | | |
| A-003 | P-004 without catalyst, | 6.95 | 34.75 | 34.12 | 31.03 |
| | 4 weeks storage | | | | |
| A-004 | P-004 with catalyst | 0.87 | 4.35 | 3.72 | 33.06 |
| A-004 | P-004 with catalyst, | 5.13 | 25.65 | 25.02 | 33.06 |
| | 1 week storage | | | | |
| A-004 | P-004 with catalyst, | 6.97 | 34.85 | 34.22 | 33.06 |
| | 4 weeks storage | | | | |
| A-007 | P-005 with catalyst | 14.81 | 74.05 | 73.42 | 75.43 |
| A-008 | P-008 with catalyst | 1.36 | 6.80 | 6.17 | 13.06 |
| A-009 | P-009 with catalyst | 2.8 | 14.00 | 13.37 | 23.54 |

*) A surface charge of 0.63 μmol/g sample was determined for the untreated fabric.
**) The surface charge in mol may be converted to coulombs by multiplying by the Faraday constant: C = 96485.309 C/mol.

### A-001, A-002:

The permanence of the finishing layers using P-002 was good. However, the available negative surface charge was unsatisfactory. The finishing layer did not absorb moisture, so that the effective available surface charge was not improved.

### A-003, A-004:

Immediately after finishing the fabric samples, the analytically detectable surface charge was only 3.7 μmol/g sample, compared to the theoretically possible 33 μmol/g sample. However, after a storage period of one week, the surface charge increased to 25 μmol/g sample, and after four weeks, with a surface charge of 34 μmol/g sample, was completely accessible. Compared to A-001 and A-002, the negative sulfonate groups of the P-004 polymer promote the swelling capability of the finishing layer. However, this layer should have more rapid swelling. The permanence of the finishing layer was satisfactory; as expected, use of the catalyst for the acid-catalyzed fixing improved the permanence.

A-007:

As the result of its rapid swelling capability, the polymer ensures immediate accessibility of the charge carriers after finishing. However, the permanence of the finish was unsatisfactory, which may possibly be due to the voluminous mPEG 1000 methacrylate monomers. In this case, the permanence may be improved by suitable optimization with regard to the crosslinking agent and the fixing parameters.

A-008, A-009:

The surface charges were not completely accessible after finishing the textile product; however, the permanence of A-009 was better than A-007.

By optimizing the fractions of hydrophilic monomers, a compromise may be found between the permanence of a finishing layer according to the invention and the rate at which the finishing layer is able to absorb water after the finishing, thus making the surface charges accessible.

### Test Series 2

Finishing solutions containing various polymer compounds of screening series P-018 through P-053 were tested for the finishing of fabric. For this purpose, the PA fabric was immersed in the impregnation batch, pressed in the Foulard process, dried in a laboratory dryer, and condensed. p-Toluenesulfonic acid was used as catalyst for the crosslinking. The wash permanence of the polymers was then investigated by charge titration (CAS).

### Fabric:

A fabric made of 100% polyamide (PA) was used in each finishing test (prefixed charmeuse, weight per unit area 135 g/m²; Fussenegger Textilveredelung GmbH, AT-6850 Dornbirn; Tricot manufacturer: Huber Tricot GmbH, AT-6841 Mäder; Prod. No. 11065).

### Finishing Bath:

The aqueous bath in each case was composed of 13.2 g polymer solution and 10.8 g catalyst solution. The polymer solution corresponded to the reaction solutions in the polymerization tests, diluted with water to a polymer concentration of 12.0% (see Section A). The catalyst solution was composed of 2.44 g p-toluenesulfonic acid monohydrate (98.5%, Fluka, M=190.22 g/mol, MP=103-105° C.) and 997.56 g water.

Finishing:

The reaction solution (diluted to 12.0% polymer fraction) was diluted with catalyst solution in a glass dish, with stirring. In the resulting bath, for each impregnation batch six fabric samples (punched out to a 11×11 cm size) were dipped by hand into the particular impregnation solution for several minutes, and pressed in the Foulard process one time. The liquor pickup relative to the dry weight was between 57% and 64 wt.-%, which corresponds to a coating quantity of 4 wt.-% polymer on average. This was followed by drying (circulation air temperature 100° C., 3 min) and condensing/fixing of the finish (circulation air temperature 150° C., 5 min).

Permanence of the Finish:

To test the washing machine permanence of the finish, in each case a finished PA fabric sample was cut in half and subjected to a machine wash (MW). A commercially available European household washing machine was loaded with a laundry bag containing the finished fabric samples in addition to polyester and polyamide samples (total quantity 2 kg). Approximately 15 g laundry detergent (Perwoll wool and fine fabric detergent) was used for the main wash cycle. After 1 machine wash at 60° C. (for undergarments) for 50-55 min, the laundered fabric samples were air-dried at room temperature and stored for at least 24 h in a standardized climate (20±2° C., 65±5% relative humidity).

Surface Charge of the Finishing Layer:

Sample preparation and charge titration using the charge analyzing system (CAS) were the same as in test series 1. The remaining coating quantity was determined based on the consumption of titration solution after subtracting the blank value for untreated fabric.

TABLE
Relative charge permanence after 1 machine wash

| | | | |
|---|---|---|---|
| | | | Relative charge |
| | | Finish coating, | permanence after |
| | | calculated from bath | 1 MW, standardized |
| | | content before | to 4 wt.-% coating |
| Finish | Polymer | drying/wt.-% | prior to MW |

Screening series 2.1—polymer
with 0% crosslinking agent

| | | | |
|---|---|---|---|
| A-018 | P-018 | 4.19 | 2 |
| A-019 | P-019 | 4.25 | 3 |
| A-020 | P-020 | 4.19 | 2 |
| A-021 | P-021 | 4.09 | 2 |
| A-022 | P-022 | 4.05 | 1 |
| A-023 | P-023 | 4.18 | 1 |
| A-024 | P-024 | 3.78 | 1 |
| A-025 | P-025 | 4.07 | 2 |
| A-026 | P-026 | 3.85 | 1 |

Screening series 2.2—polymer
with 5% crosslinking agent

| | | | |
|---|---|---|---|
| A-027 | P-027 | 3.95 | 35 |
| A-028 | P-028 | 3.92 | 35 |
| A-029 | P-029 | 4.07 | 29 |
| A-030 | P-030 | 4.09 | 32 |
| A-031 | P-031 | 3.94 | 16 |
| A-032 | P-032 | 3.91 | 19 |
| A-033 | P-033 | 3.89 | 23 |
| A-034 | P-034 | 3.73 | 14 |
| A-035 | P-035 | 3.79 | 15 |

Screening series 2.3—polymer
with 10% crosslinking agent

| | | | |
|---|---|---|---|
| A-036 | P-036 | 4.18 | 21 |
| A-037 | P-037 | 4.10 | 38 |
| A-038 | P-038 | 4.02 | 37 |
| A-039 | P-039 | 3.94 | 36 |
| A-040 | P-040 | 3.91 | 36 |
| A-041 | P-041 | 3.85 | 35 |
| A-042 | P-042 | 3.84 | 27 |
| A-043 | P-043 | 3.85 | 27 |
| A-044 | P-044 | 3.85 | 29 |

Screening series 2.4—polymer
with 15% crosslinking agent

| | | | |
|---|---|---|---|
| A-045 | P-045 | 4.09 | 41 |
| A-046 | P-046 | 4.13 | 42 |
| A-047 | P-047 | 4.12 | 32 |
| A-048 | P-048 | 3.87 | 36 |
| A-049 | P-049 | 3.82 | 39 |
| A-050 | P-050 | 3.83 | 28 |
| A-051 | P-051 | 3.85 | 35 |
| A-052 | P-052 | 3.85 | 35 |
| A-053 | P-053 | 3.94 | 14 |

Test Series 3

Using the same test procedure as in test series 2, various finishing solutions were tested for wash permanence, using the polymer solutions of crosslinking agent screenings P-054 through P-059.

TABLE
Relative charge permanence after 1 machine wash

| | | | Relative charge |
|---|---|---|---|
| | | Finish coating, | permanence after |
| | | calculated from bath | 1 MW, standardized |
| | | content before | to 4 wt.-% coating |
| Finish | Polymer | drying/wt.-% | prior to MW |
| A-054 | P-054 | 3.84 | 7 |
| | (1% NMBA) | | |
| A-055 | P-055 | 4.18 | 13 |
| | (equimolar NMA) | | |
| A-056 | P-056 | 4.05 | 8 |
| | (equimolar GMA) | | |
| A-057 | P-057 | 3.96 | 18 |
| | (equimolar | | |
| | p-EMKO . . .) | | |
| A-058* | P-058 | 3.98, 4.07 | 7, 9 |
| | (equimolar EMKO | | |
| | alkenyl . . .) | | |
| A-059 | P-059 | 3.62 | 19 |
| | (NBMA, ETMA) | | |

*Multiple test results

Summary:

The polyelectrolyte consumption in screening series A-054 through A-059, using different crosslinking agents (crosslinking agent contents in each case based on an equimolar quantity of 1% NBMA used), showed different charge accessibilities in the unlaundered state. However, the polyDADMAC consumption for the laundered fabric samples (1 MW at 60° C. for 50-55 min) was in the range of 1 mL 0.001N polyDADMAC for all layers. It was not possible to achieve any improvement in the washing machine permanence by using the different crosslinking agents, or by replacing mPEG 350 methacrylate with ethyl triglycol methacrylate (A-059).

### Test Series 4

Using the same test procedure as in test series 2, various finishing solutions were tested for wash permanence, using the polymer solutions of counterion screenings P-060 through P-064. The permanence after 1 and 5 machine washes was tested.

TABLE
Relative charge permanence after 1 and 5 machine washes

| | | | Relative charge | |
|---|---|---|---|---|
| | | | permanence, | |
| | | Finish coating, | standardized to 4 | |
| | | calculated from bath | wt.-% coating | |
| | | content before | prior to MW | |
| Finish | Polymer | drying/wt.-% | 1 MW | 5 MW |
| A-060 | P-060 | 4.22, 4.13 | 21 | 14 |
| | (lithium) | | | |
| A-061 | P-061 | 4.15, 3.97 | 99 | 91 |
| | (ammonium) | | | |
| A-062 | P-062 | 3.97, 4.11 | 56 | 86 |
| | (triethylammonium) | | | |
| A-063 | P-063 | 4.00, 4.04 | 51 | 60 |
| | (1-methyl- | | | |
| | imidazolium) | | | |
| A-064 | P-064 | 3.91, 4.07 | 73 | 136 |
| | (4-methyl- | | | |
| | morpholinium) | | | |

### Summary:

The polyelectrolyte consumption in screening series A-060 through A-064, using different AMPS counterions, was between 2 mL and 4 mL 0.001 N polyDADMAC, with the exception of A-060 with AMPS lithium, for which the consumption in the unlaundered state was approximately 8.5 mL 0.001 N polyDADMAC. The values for the relative charge permanence increased due to the lower swelling capability of the donor layers. The polyDADMAC consumption for the laundered fabric samples (1 and 5 MW at 60° C. for 50-55 min) was in the same range as for screening series A-027 through A-053, so that it was likewise not possible to determine any significant improvement regarding the charge accessibility.

### Test Series 5

Using the same test procedure as in test series 2, various finishing solutions were tested for wash permanence after 1 and 5 machine washes.

For the tests, the finishing formulations according to the invention, together with a polyurethane dispersion (Lamethan NKS-AF) in combination with an alkyl-modified melamine/formaldehyde derivative (Knittex CHN) as a binder system, were tested. On the one hand it was thus possible to further increase the layer permanence of the donor layer, and on the other hand it could thus be demonstrated that the polymer compounds according to the invention are mixable and crosslinkable with other polymers.

Finishing baths A-065, A-066: Same as in test series 2. Finishing bath A-067: The aqueous bath was composed of 13.2 g polymer solution, 2.6 g Dicrylan PGS (7753) (60%, Erba AG, d=1.10 g/mL), 0.2 g Knittex CHN (Erba AG, d=1.18 g/mL), and 8.0 g catalyst solution. Finishing bath A-068: The aqueous bath was composed of 13.2 g polymer solution, 3.3 g Lamethan NKS-AF (48%, CHT R. Beitlich GmbH, d=1.0 g/mL), 0.2 g Knittex CHN, 7.2 g catalyst solution, and 0.1 g p-toluenesulfonic acid monohydrate. The polymer solution corresponded to the reaction solutions in the polymerization tests, diluted with water to a polymer concentration of 12.0% (see Section A). The catalyst solution was the same as in test series 2.

TABLE
Relative charge permanence after 1 and 5 machine washes

| | | | Relative charge | |
|---|---|---|---|---|
| | | | permanence after | |
| | | Finish coating, | 1 MW, standardized | |
| | | calculated from bath | to 4 wt.-% coating | |
| | | content before | prior to MW | |
| Finish | Polymer | drying/wt.-% | 1 MW | 5 MW |
| A-065 | P-065 | 3.91 | 3 | |
| A-066 | P-066 | 3.93, 3.93 | 69 | 27 |
| A-067 | P-042, | 4.24, 4.09 | 51 | 66 |
| | Dicrylan PGS, | | | |
| | Knittex CHN | | | |
| A-068 | P-042, Lamethan | 3.97, 3.84 | 68 | 77 |
| | NKS-AF, | | | |
| | Knittex CHN | | | |

Summary:

The polyelectrolyte consumption in tests A-065 through A-068 in the unlaundered state was approximately 5 mL 0.001 N polyDADMAC. The polyDADMAC consumption for laundered fabric sample A-065 (1 MW at 60° C. for 50-55 min), using N,N′-methylene-bis-acrylamide (MBAm) as crosslinking agent, was approximately 0.4 mL 0.001 N polyDADMAC, which was in the range of screening series A-018 through A-026. However, when the previous NBMA crosslinking agent was combined with MBAm (A-066), for the first time the polyDADMAC consumption for the laundered textile increased to just under 3.5 mL 0.001N polyDADMAC. The relative charge permanence of 69% was also good. However, this value dropped to 27% after 5 MW. The relative charge permanence could be maintained, even after five launderings, by combining the polymer with the Dicrylan PGS/Knittex CHN (A-067) and Lamethan NKS-AF/Knittex CHN (A-068) binder systems.

### Test Series 6

Using the same test procedure as in test series 5, various finishing solutions were tested for wash permanence after 1 and 5 machine washes. An oxime-blocked polyisocyanate crosslinking agent (Phobol XAN) was used instead of the formaldehyde-containing Knittex CHN crosslinking agent in order to avoid formaldehyde in the donor layer.

Finishing bath A-069: The aqueous bath was composed of 13.2 g polymer solution, 2.6 g Dicrylan PGS (7753) (60%, Erba AG, d=1.10 g/mL), 0.2 g Phobol XAN (Erba AG, d=1.03-1.08 g/mL), and 8.0 g catalyst solution. Finishing bath A-070: The aqueous bath was composed of 13.2 g polymer solution, 3.3 g Lamethan NKS-AF (48%, CHT R. Beitlich GmbH, d=1.0 g/mL), 0.2 g Phobol XAN, 7.2 g catalyst solution, and 0.1 g p-toluenesulfonic acid monohydrate.

TABLE
Relative charge permanence after 1 and 5 machine washes

| | | | Relative charge | |
|---|---|---|---|---|
| | | | permanence after | |
| | | Finish coating, | 1 MW, standardized | |
| | | calculated from bath | to 4 wt.-% coating | |
| | | content before | prior to MW | |
| Finish | Polymer | drying/wt.-% | 1 MW | 5 MW |
| A-069 | P-042, Dicrylan | 4.05, 3.93 | 37 | 43 |
| | PGS, Phobol XAN | | | |
| A-070 | P-042, Lamethan | 4.10, 4.09 | 83 | 96 |
| | NKS-AF, | | | |
| | Phobol XAN | | | |

### Summary:

Tests A-069 and A-070 showed that the Knittex CHN crosslinking agent could be replaced with the formaldehyde-free polyisocyanate crosslinking agent Phobol XAN without any problems.

### Test Series 7

Using the same test procedure as in test series 5, various finishing solutions were tested for wash permanence. To increase the swelling capability and therefore the charge accessibility of the layer, the fractions of additional polymer were reduced.

Finishing bath A-071: The aqueous bath was composed of 79.2 g polymer solution, 7.8 g Dicrylan PGS (7753), 1.2 g Phobol XAN, and 55.8 g catalyst solution. A-072: The aqueous bath was composed of 79.2 g polymer solution, 9.9 g Lamethan NKS-AF, 1.2 g Phobol XAN, 53.3 g catalyst solution, and 0.4 g p-toluenesulfonic acid monohydrate; A-071: The aqueous bath was composed of 13.2 g polymer solution, 0.7 g Dicrylan PGS (7753), 0.2 g Phobol XAN, and 9.9 g catalyst solution. A-074: The aqueous bath was composed of 13.2 g polymer solution, 0.8 g Lamethan NKS-AF, 0.2 g Phobol XAN, 9.7 g catalyst solution, and 0.1 g p-toluenesulfonic acid monohydrate. A-075: The aqueous bath was composed of 13.2 g polymer solution, 0.2 g Phobol XAN, and 10.6 g catalyst solution.

TABLE
Relative charge permanence

| | | | |
|---|---|---|---|
| | | | Relative charge |
| | | Finish coating, | permanence in %, |
| | | calculated from bath | standardized to 4 |
| | Number | content before | wt.-% coating |
| Finish | of MWs | drying/wt.-% | prior to MW |

Polymer: P-042 scale-up No. 2,
Dicrylan PGS (one-half of A-069), Phobol XAN

| | | | |
|---|---|---|---|
| A-071 | 1 | 4.14, 3.93 | 55, 53 |
| | 5 | 4.09 | 51 |
| | 25 | 4.06 | 26 |
| | 50 | 3.85 | 18 |

Polymer: P-042 scale-up No. 2,
Lamethan NKS-AF (one-half of A-070), Phobol XAN

| | | | |
|---|---|---|---|
| A-072 | 1 | 4.07 | 71 |
| | 5 | 4.05 | 78 |
| | 25 | 3.97 | 64 |
| | 50 | 3.64 | 30 |

Polymer: P-042 scale-up No. 2,
Dicrylan PGS (one-fourth of A-069), Phobol XAN

| | | | |
|---|---|---|---|
| A-073 | 1 | 4.05 | 60 |
| | 5 | 4.05 | 61 |
| | 25 | 4.05 | 45 |
| | 50 | 4.13 | 11 |

Polymer P-042 scale-up No. 2,
Lamethan NKS-AF (one-fourth of A-070), Phobol XAN

| | | | |
|---|---|---|---|
| A-074 | 1 | 4.32 | 70 |
| | 5 | 4.11 | 62 |
| | 25 | 4.12 | 58 |
| | 50 | 3.80 | 22 |

Polymer: P-042 scale-up No. 2, Phobol XAN

| | | | |
|---|---|---|---|
| A-075 | 1 | 4.16 | 64 |
| | 5 | 4.20 | 63 |
| | 25 | 4.00 | 29 |
| | 50 | 4.17 | 15 |

### Summary:

For A-071, the content of Dicrylan PGS was reduced to one-half that of A-069 in order to increase the swelling capability and therefore the charge accessibility of the layer. As a result, the polyelectrolyte consumption for the unlaundered fabric samples increased to 9 mL 0.001 N polyDADMAC. It was likewise possible to increase the polyDADMAC consumption for the laundered fabric samples (1 and 5 MW at 60° C. for 50-55 min) to almost 5 mL 0.001 N polyDADMAC, resulting in a relative charge permanence of 53%. A decline in the relative charge permanence to 26% and 18% occurred only after 25 and 50 MW, respectively. The content of Lamethan NKS-AF was similarly reduced for A-072, resulting in permanence comparable to A-071. For A-073, reducing Dicrylan PGS to one-fourth the quantity originally used resulted in no significant improvement in the determined layer parameters. For A-074, the content of Lamethan NKS-AF was likewise reduced to one-fourth the quantity originally used, resulting in permanence comparable to A-072. Interestingly, for A-075 it was determined that, without any use of Dicrylan PGS or Lamethan NKS-AF, but using the NBMA crosslinking agent incorporated into the polymer according to the invention together with 1% Phobol XAN, it was possible to achieve permanence comparable to that in tests A-071 through A-074.

### Test Series 8

Using the same test procedure as in test series 5, various finishing solutions were tested for wash permanence.

To further optimize the composition of the finishing formulation according to the invention, based on A-072 the further tests were carried out using Lamethan NKS-AF instead of Dicrylan PGS. Although Lamethan NKS-AF and Dicrylan PGS showed comparable wash permanence in the previous tests, Lamethan NKS-AF is lower in cost, is more elastic, and has a better, i.e., more pleasant, touch (feel). On the other hand, Dicrylan PGS is known for its high hydrolysis resistance.

Finishing bath: The aqueous bath in each case was composed of 13.2 g polymer solution, 1.7 g Lamethan NKS-AF, 0.2 g Phobol XAN, 8.8 g catalyst solution, and 0.1 g p-toluenesulfonic acid monohydrate.

TABLE
Relative charge permanence

| | | | |
|---|---|---|---|
| | | | Relative charge |
| | | Finish coating, | permanence in %, |
| | | calculated from bath | standardized to 4 |
| | Number | content before | wt.-% coating |
| Finish | of MWs | drying/wt.-% | prior to MW |

P-036, Lamethan NKS-AF, Phobol XAN

| | | | |
|---|---|---|---|
| A-076 | 1 | 4.23 | 113 |
| | 5 | 4.44 | 125 |

P-044, Lamethan NKS-AF, Phobol XAN

| | | | |
|---|---|---|---|
| A-077 | 1 | 4.07 | 75 |
| | 5 | 4.10 | 71 |
| | 25 | 4.01 | 86 |
| | 50 | 3.95 | 55 |

P-033, Lamethan NKS-AF, Phobol XAN

| | | | |
|---|---|---|---|
| A-078 | 1 | 3.94 | 55 |
| | 5 | 3.89 | 61 |
| | 25 | 4.51 | 35 |
| | 50 | 3.93 | 13 |

P-024, Lamethan NKS-AF, Phobol XAN

| | | | |
|---|---|---|---|
| A-079 | 1 | 4.03 | 27 |
| | 5 | 4.02 | 24 |

Based on test A-072, mPEG 350 methacrylate was dispensed with for A-076, but 40% HEMA was used. In contrast, 40% mPEG 350 methacrylate and 0% HEMA were used for A-077. Good relative charge permanence was achieved for both finishes, although with a polyelectrolyte consumption in the range of 2.5 mL 0.001 N polyDADMAC, the swelling capability of the layer was greatly reduced for A-076. For A-077, on the other hand, it was possible to increase the polyDADMAC consumption to greater than 6 mL 0.001 N polyDADMAC for the laundered fabric samples (1, 5, and 25 MW at 60° C. for 50-55 min). Even after 50 MW, the consumption was still 5 mL 0.001 N polyDADMAC. A-077 was therefore the best finishing formulation thus far, with a high swelling capability as well as high relative charge permanence. To increase the swelling capability and therefore the charge accessibility of the layer, for A-078, likewise based on A-072, the content of NBMA crosslinking agent incorporated into the polymer was decreased to 5%, although this reduced the permanence compared to A-072, in particular after 25 and 50 MW. In A-079, polymer P-024 contained no NBMA, but, the same as P-042, contained 10% HEMA, as the result of which the oxime-blocked polyisocyanate Phobol XAN was likewise able to crosslink, thus ensuring that the permanence was maintained.

### Test Series 9

Using the same test procedure as in test series 5, various finishing solutions were tested for wash permanence. The formulations corresponded to A-072, containing various fractions of Phobol XAN.

Finishing bath: The aqueous bath in each case was composed of 13.2 g polymer solution; 1.7 g Lamethan NKS-AF; 0.2 g (A-080), 0.1 g (A-081), 0.0 g (A-082), or 0.7 g (A-083) of Phobol XAN; 8.7 g (A-080), 8.9 g (A-081), 9.0 g (A-082), or 8.3 g (A-083) of catalyst solution; and 0.1 g p-toluenesulfonic acid monohydrate.

TABLE
Relative charge permanence

| | | | |
|---|---|---|---|
| | | | Relative charge |
| | | Finish coating, | permanence in %, |
| | | calculated from bath | standardized to 4 |
| | Number | content before | wt.-% coating |
| Finish | of MWs | drying/wt.-% | prior to MW |

P-042 scale-up No. 2, Lamethan NKS-AF, 1.5% Phobol XAN

| | | | |
|---|---|---|---|
| A-080 | 1 | 4.05 | 71 |
| | 5 | 4.08 | 72 |
| | 25 | 3.89 | 81 |
| | 50 | 4.02 | 39 |

P-042 scale-up No. 2, Lamethan NKS-AF, 0.5% Phobol XAN

| | | | |
|---|---|---|---|
| A-081 | 1 | 4.21 | 99 |
| | 5 | 4.05 | 95 |
| | 25 | 4.09 | 93 |
| | 50 | 4.01 | 34 |

P-042 scale-up No. 2, Lamethan NKS-AF, no Phobol XAN

| | | | |
|---|---|---|---|
| A-082 | 1 | 4.13 | 72 |
| | 5 | 4.06 | 69 |
| | 25 | 4.02 | 45 |
| | 50 | 3.83 | 21 |

P-026, Lamethan NKS-AF, 3% Phobol XAN

| | | | |
|---|---|---|---|
| A-083 | 1 | 4.11 | 21 |
| | 5 | 4.20 | 16 |

Based on test A-072, the content of the NBMA crosslinking agent incorporated into the polymer was reduced to 0% for A-079. As expected, this adversely affected the permanence. For A-080 and A-081, likewise based on A-072, the crosslinking agent content of Phobol XAN was changed to 1.5% and 0.5% Phobol XAN, respectively. As a result, hardly any difference from A-072 was detectable. Based on test A-072, the crosslinking agent content was reduced to 0% Phobol XAN for A-082, resulting in the interesting finding (see test A-075) that either Phobol XAN, Dicrylan PGS binder, or Lamethan NKS-AF binder could be dispensed with. In A-083, P-026 was used with 3% Phobol XAN, and without HEMA and without NBMA crosslinking agent. The test showed no improvement over the previous results.

### Test Series 10

Production test based on A-077 and A-080 through A-082 for anionic fabric impregnation, using a 15% polymer dispersion of P-044 (scale-up No. 2). According to the permanence tests for A-080 through A-082, the use of Phobol XAN as oxime-blocked polyisocyanate crosslinking agent was dispensed with, since it showed no significant influence on the permanence. The fabrics were padded, and dried and condensed directly in the laboratory clamping frame. Magnesium chloride and tartaric acid were used as catalysts to assist in the crosslinking. The wash permanence of the polymers was then investigated by charge titration (CAS).

Fabric:

PA: 100% PA the same as in test series 1 and 2. PES: 100% PES, Christian Eschler AG, CH-9055 Baler, eyelet (circular knitted fabric), 130-145 g/m², color 100020 washed out. PES/PUE: 83% PES/17% PUE (Elastan), Christian Eschler AG, CH-9055 Bühler, charmeuse (warp knit fabric), 130-140 g/m², color 100202, continuously washed out. CO: 100% CO, knitted fabric, Greuter-Jersey AG, CH-8583 Sulgen, 167 g/m².

Catalyst stock solution: ≧98.0% 125 g magnesium chloride×6H₂O, Fluka; ≧99.5% 50 g L-(+) tartaric acid, Fluka; 2325 g water (deionized).

### Fabric Impregnation Batch:

1320 g of P-044 scale-up No. 2 reaction solution; 210 g Lamethan NKS-AF, CHT R. Beitlich GmbH. 1170 g water (not deionized); 300 g catalyst stock solution. The catalyst was not added until immediately before the padding.

### Finishing:

After preparation, the finishing bath was thoroughly stirred and filled into the Foulard immersion trough. In each case, 6 linear meters were then padded under production conditions (4.5 bar roller pressure, 4 m/min), and subsequently dried/condensed directly in the laboratory clamping frame (170° C., 2 min). To improve the touch of the material, after the finishing the finished fabric was treated in a clothes dryer (tumbler) for 60 min. In each case three fabric samples (cut to 37×50 cm size) were then padded, and the average liquor pickup was determined relative to the dry weight. Washing machine permanence: Same as in test series 2. Charge titration: Sample preparation and charge titration using the charge analyzing system (CAS) were the same as in test series 1 and 2.

TABLE
Relative charge permanence

| | | | | Relative charge |
|---|---|---|---|---|
| | | | Finish coating, | permanence in %, |
| | | | calculated from bath | standardized to 4 |
| | | Number | content before | wt.-% coating |
| Finish | Fabric | of MWs | drying/wt.-% | prior to MW |
| A-084-I | 100% PA | 1 | 3.69 | 80 |
| A-084-II | | 5 | 3.69 | 106 |
| A-084-III | | 25 | 3.69 | 106 |
| A-084-IV | | 50 | 3.69 | 104 |
| A-084-V | | 100 | 3.69 | 106 |
| A-085-I | 100% PES | 1 | 4.82 | 48 |
| A-085-II | | 5 | 4.82 | 55 |
| A-085-III | | 25 | 4.82 | 45 |
| A-085-IV | | 50 | 4.82 | 40 |
| A-085-V | | 100 | 4.82 | 21 |
| A-086-I | 83% PES/ | 1 | 3.48 | 61 |
| A-086-II | 17% PUE | 5 | 3.48 | 56 |
| A-086-III | | 25 | 3.48 | 63 |
| A-086-IV | | 50 | 3.48 | 73 |
| A-086-V | | 100 | 3.48 | 106 |
| A-087-I | 100% CO | 1 | 5.56 | 103 |
| A-087-II | | 5 | 5.56 | 109 |
| A-087-III | | 25 | 5.56 | 139 |
| A-087-IV | | 50 | 5.56 | 164 |
| A-087-V | | 100 | 5.56 | 174 |

### Summary:

The average liquor pickup of the fabric samples was 55% for PA (A-084), 72% for PES (A-085), 52% for PES/PUE (A-086), and 83% for CO (A-087). Different coatings were thus achieved, namely, approximately 3.7% polymer coating on the PA fabrics, approximately 4.8% polymer coating on the PES fabrics, approximately 3.5% polymer coating on the PES/PUE fabrics, and approximately 5.6% polymer coating on the CO fabrics.

### Comparison of Production Tests,

A-084 through A-087: Production test A-084 showed excellent charge accessibility as well as relative charge permanence, which allowed a high active substance loading capacity. The polyelectrolyte consumption for the unlaundered fabric samples was 7 mL 0.001 N polyDADMAC. The polyDADMAC consumption for the laundered fabric sample (1 MW at 60° C. for 50-55 min) was 5.5 mL 0.001 N polyDADMAC. The consumption after 5, 25, 50, and 100 MW increased to almost 8 mL 0.001 N polyDADMAC, as occasionally caused by washing out of uncrosslinked polymer fractions. As a result, the relative charge permanence increased to greater than 100%. Production test A-085 was likewise resistant for more than 100 washes. However, due to the lack of anchor groups in the polyester, much less donor layer can be applied, as evidenced by polyelectrolyte consumption in the range of 2 mL 0.001 N polyDADMAC for the laundered fabric samples. The relative charge permanence was approximately 45%. Production test A-086 was likewise resistant for more than 100 washes. The polyelectrolyte consumption for the unlaundered fabric samples (1, 5, and 25 MW at 60° C. for 50-55 min) was 4 mL 0.001 N polyDADMAC. After 50 and 100 MW, the polyDADMAC consumption increased to 5 mL and 7 mL, respectively. Apparently, the elasticity of the donor layer increases over time, thus also increasing the analytically detectable surface charge. Production test A-087 was also resistant for more than 100 washes. In this case, the same as for A-086, the charge accessibility increased with an increasing number of swelling cycles. After 100 MW, at 11.4 mL the polyDADMAC consumption reached the maximum theoretically possible consumption of 11.6 mL 0.001 N polyDADMAC. Therefore, the relative charge permanence of 174% after 100 MW for CO corresponds to the absolute layer permanence, since none of the donor layer is lost during laundering.

### Test Series 11

Production test based on A-084 through A-087. Impregnation with a 15% polymer dispersion of P-067 (scale-up). Compared to P-044, which contained 40% mPEG 350 methacrylate and 20% 2-ethylhexyl acrylate, P-067 contained only 20% mPEG 350 methacrylate, but also 40% 2-ethylhexyl acrylate, and was therefore much more lipophilic.

The test procedure was analogous to test series 3. Fabric was analogous to test series 1 and 2, and catalyst stock solution was analogous to test series 3.

### Fabric Impregnation Batch:

330 g P-067 scale-up reaction solution; 53 g Lamethan NKS-AF, CHT R. Beitlich GmbH, 292 g water (not deionized); 75 g catalyst stock solution. The catalyst was not added until immediately before the padding.

Washing machine permanence: Same as in test series 2. Charge titration: Sample preparation and charge titration using the charge analyzing system (CAS) were the same as for test series 1 and 2.

TABLE
Relative charge permanence

| | | | | Relative charge |
|---|---|---|---|---|
| | | | Finish coating, | permanence in %, |
| | | | calculated from bath | standardized to 4 |
| | | Number | content before | wt.-% coating |
| Finish | Fabric | of MWs | drying/wt.-% | prior to MW |
| A-088-I | 100% PA | 1 | 3.36 | 54 |
| A-088-II | | 5 | 3.36 | 71 |
| A-088-III | | 25 | 3.36 | 73 |
| A-088-IV | | 50 | 3.36 | 74 |
| A-088-V | | 100 | 3.36 | 59 |

### Summary:

The average liquor pickup of the fabric samples was in the range of 49%, thus achieving a coating of approximately 3.4% polymer on the PA fabrics. Production test A-088, the same as A-084, had excellent charge accessibility. Due to the lipophilic character of the amphiphilic donor layer, the polyelectrolyte consumption of the laundered fabric samples (1, 5, 25, 50, and 100 MW at 60° C. for 50-55 min), at approximately 6 mL 0.001 N polyDADMAC, was somewhat less than A-084. The relative charge permanence was in the range of 70%.

### C. Cytotoxicity Test

To test the tolerance of finished textiles according to the invention, a fabric sample was subjected to a cytotoxicity test according to DIN EN ISO 10993-5 (“Biological Evaluation of Medical Devices, Part 5: Testing of in vitro cytotoxicity”). The fabric sample was 100% PA fabric which had been finished with finishing formulation No. A-084. The fabric sample was laundered beforehand one time (60° C. for 50-55 min). The fabric was not loaded with emulsion or active substances.

The cytotoxicity testing according to DIN EN ISO 10993-5 is a recognized requirement for all medical devices. By use of cell cultures, it is possible to detect toxic substances which may leach out of the tested products. The release of toxic substances from a textile product with skin contact is a prerequisite for development of skin irritation. Cytotoxicity testing allows assessment of a hazard potential for skin irritation, which is detected as a sum parameter.

### Method:

Cell line: L 929 cells (ATCC No. CCL1, NCTC clone 929 L (DSMZ)), number of passages: 31. Culture medium: DMEM with 10% FCS. Extraction method: Incubation of the test specimen with acidic perspiration solution according to DIN EN ISO 105-E04 for 24 h at 37° C., with light shaking; the perspiration solution was adjusted to pH 7.3-7.4 and sterile-filtered. Incubation of the cell culture: 68-72 h with perspiration solution in dilution steps of 33, 3% to 9, and 9%. Cytotoxicity testing: After the cells were incubated, the protein content was compared to that of the controls as a measure of the cellular growth (Biol. Toxicol. 1984, 1(1), 55-65). Positive and negative controls were also included in the experiment in order to verify the validity of the test system. Cells exhibit altered proliferation and division rates in the presence of cytotoxic substances (growth inhibition test). Test material: Concentrations of the test material in the culture medium: 9.9%, 14.8%, 22.2%, and 33.3%.

### Results:

No discoloration was visually observed in the perspiration extract. A slightly sweet odor was detected from the sample material, i.e., perspiration extract. An increase in the pH of the perspiration extract from 5.5 to 6.0 was observed. The following values were obtained:

| Concentrated test | Growth |
|---|---|
| material/%-vol. | inhibition/% |
| 33.3 | 18 |
| 22.2 | 13 |
| 14.8 | 10 |
| 9.0 | 9 |

According to DIN EN ISO 10993-5, growth inhibition of greater than 30% compared to a solvent control is regarded as a cytotoxic effect. Using the described test methodology, the perspiration extract of the sample showed growth inhibition of 18% in the cytotoxicity test. Dose-dependent growth inhibition of the L929 cells was observed for the test material, but the significance threshold of 30% was not exceeded. Based on the assessment criteria, the sample showed no biological activity. It was therefore concluded that under the stated conditions, no cytotoxic substances were released from the test specimen that could lead to irritation upon skin contact.

### D. Emulsions

To allow efficient application of the usually lipophilic active substances from a diluted emulsion to the negatively charged finishing layer according to the invention, an emulsifier having corresponding positive charges was used. This resulted in active substance emulsions with oil phase droplets containing active substance, which due to the positive surface charge that is present are easily incorporated into the matrix of the finishing layer according to the invention which is provided with negative charge sites. After the finishing layer is loaded with the emulsion, the active substance is able to diffuse from the emulsified oil droplets into the lipophilic regions of the polymer matrix. The active substance may be subsequently delivered from the oil phase of the adsorbed emulsion particles and/or the lipophilic phases of the polymer matrix.

Phosphatidylcholine lecithins, for example 1-palmityl-2-oleyl-sn-glycero-3-phosphatidylcholine (palmityl oleyl phosphatidylcholine (POPC)), are suitable for use according to the invention. Due to the amine groups, the positive charge at the surface of the emulsified oil droplets interacts with the negative charge sites of the polymer finishes according to the invention.

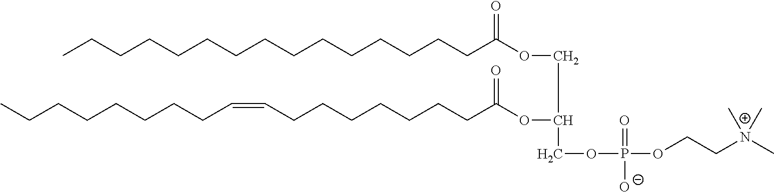 [SMILES: CCCCCCCC/C=C\CCCCCCCC(=O)OC(COC(=O)CCCCCCCCCCCCCCC)COP(=O)([O-])OCC[N+](C)(C)C]

Lecithin-containing oil/water microemulsions are known. Reference is made to DE 198 59 427 A1, for example. As a rule, a macroemulsion is converted to a microemulsion or miniemulsion by high-pressure homogenization.

In addition, behenyl trimethylammonium methosulfate, for example, is suitable for microemulsions:

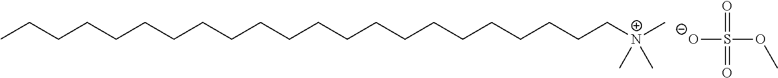

As a co-emulsifier, behenyltrimethyl ammonium methosulfate in combination with cetyl alcohol

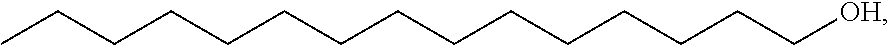 [SMILES: CCCCCCCCCCCCCCCO]

containing 25 wt.-% BTMS and 75 wt.-% cetyl alcohol, is available as Varisoft BTMS flakes (Evonik Goldschmidt GmbH). In the present case, however, cetyl alcohol has the tendency to settle out during loading of the finishing layer according to the invention.

Ethyl-N-alpha-lauroyl-L-arginate HCl (referred to below as lauroyl arginate) has proven to be a particularly suitable emulsifier for an emulsion according to the invention:

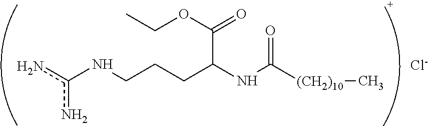

The synthesis of lauroyl arginate (CAS No. 60372-77-2) is described in WO 01/94292 A1, and the substance is available from Laboratorios Miret SA, Barcelona, Spain. Lauroyl arginate is a white, hygroscopic solid which has dispersibility of up to 247 g/kg in water. The melting point is 50° C. to 58° C.

Lauroyl arginate is used as an antimicrobial preservative, whose antimicrobial properties are based on its characteristics as a cationic surfactant. Lauroyl arginate is effective against a broad spectrum of gram-negative and gram-positive bacteria, as well as yeasts and molds. It suppresses the growth of bacterial colonies, but does not cause cell lysis. It has been recommended for authorization for use in cosmetic products (Scientific Committee on Consumer Products (SCCP), Opinion on ethyl lauroyl arginate HCl, SSCP/1106/07, Apr. 15, 2008) and in foods (Food Standards Australia New Zealand, Application A1015, Ethyl lauroyl arginate as a food additive—Assessment Report, May 6, 2009).

The use of lauroyl arginate in combination with sorbic acid, potassium sorbate, or sodium sorbate is known from WO 02/087328 A2. The antiviral activity of lauroyl arginate is postulated in WO 2008/014824 A1. Other similar antimicrobially active surfactants are known from WO 2007/014580 A 1. The disclosures of the above-cited documents are hereby considered part of the present description.

It is not known from the prior art to use lauroyl arginate as an emulsifier. The use of lauroyl arginate for this purpose has the advantage that on the one hand, no additional preservatives are needed for a corresponding emulsion. On the other hand, at the same time such an emulsion is not harmful to health.

In addition, the antimicrobial properties of the emulsion are transferred to the textile, as the result of which the textile itself acquires bacteriostatic properties. This is due to the low effective concentrations of lauroyl arginate in the μg/mL range against various microorganisms, and the relatively high usage concentration of this cationic emulsifier of up to 0.8% in the emulsion. Particularly high effectiveness of the emulsion-loaded fabric is achieved by the previously described use of lauroyl arginate in combination with sorbates or benzoates, for example, resulting in a synergistic effect of these active substances.

Evening primrose oil is one example of a carrier medium or active substance with which a finishing layer according to the invention may be loaded. The seeds of the common evening primrose (Oenothera biennis) contain approximately 7-10 wt.-% linolenic acid. The corresponding oil of the evening primrose has, for example, a pain-relieving effect for premenstrual syndrome, and has a healing effect for skin diseases such as psoriasis and neurodermatitis. Evening primrose oil consists of 71 wt.-% linoleic acid, 10 wt.-% gamma-linolenic acid, 7 wt.-% oleic acid, 2 wt.-% stearic acid, 7 wt.-% palmitic acid, and 3 wt.-% other substances. The active ingredient is gamma-linolenic acid.

Alternatively, other lipophilic media based on paraffin or paraffin oil, for example, may be used as carrier medium for active substances having lipophilic activity. Of course, these media should preferably be tolerated by the skin. Hydrophilic active substances, in turn, may be loaded within water-in-oil-in-water emulsions or liposomes on finishing layers according to the invention.

WO 2007/050580 A2 and U.S. Pat. No. 5,474,783 disclose a number of active substances that are suitable for transdermal absorption. The disclosed content of these documents forms an integral part of the present description.

In addition to the actual active substances, auxiliary substances may be provided which, for example, enhance the absorption of the active substance into the skin (so-called penetration enhancers), or which contain osmophore or chromophore groups, for example, which as fragrances or dyes appeal to the sensory demands of consumers.

In addition, suitable as further auxiliary substances or additives are all substances which, for example, prevent freezing of the emulsion or which generally ensure stability of the emulsion over a wide temperature range and period of time, in that they increase the chemical, physical, and biological stability of the emulsion. The emulsion may also contain bitter substances which discourage uncontrolled ingestion by children.

TABLE
Exemplary embodiments for emulsions

| Emulsion No. | E-018 | E-019 | E-020 | E-021 | E-022 |
|---|---|---|---|---|---|
| Refined evening primrose oil | 25.5 | 25.5 | 25.5 | 25.5 | 25.5 |
| (active substance), Carl Roth GmbH + | | | | | |
| Co. KG, Prod. No. 3691.2 [wt.-%] | | | | | |
| 97% lecithin, Carl Roth GmbH + | 2.0 | — | — | — | 1.5 |
| Co. KG, Prod. No. 9812.1 [wt.-%] | | | | | |
| Lipoid S40 (lecithin emulsion), | — | 2.0 | — | — | — |
| Lipoid GmbH, Prod. No. 740046 | | | | | |
| [wt.-%] | | | | | |
| Phosal 50 PG (contains 40% PG), | — | — | 2.0 | — | — |
| Phospholipid GmbH, Prod. | | | | | |
| No. 368214 [wt.-%] | | | | | |
| Varisoft BTMS flakes (25% BTMS, | — | — | — | 2.0*) | 2.0*) |
| 75% cetyl alcohol), Evonik | | | | | |
| Goldschmidt GmbH, Prod. No. | | | | | |
| 204109 [wt.-%] | | | | | |
| ≧99.5% propylene glycol (GC), | 6.5 | 6.5 | 6.5 | 6.5 | 6.5 |
| Fluka [wt.-%] | | | | | |
| Deionized water [ wt.-%] | 65.5 | 65.5 | 65.5 | 65.5 | 65.5 |

*)corresponds to 0.5 wt.-% cationic emulsifier

### Emulsion E-018

Preparation: 50.0 g evening primrose oil was placed in a 250-mL Erlenmeyer flask. 4.0 g lecithin was then added, with stirring. The mixture was stirred on a magnetic stirrer, and after the lecithin was uniformly swelled, 13.0 g propylene glycol and 133.0 g water were slowly added dropwise, with intensive stirring, and stirring was continued for 3 h (poor miscibility). Determination of homogeneity: 1 g emulsion solution was diluted 1:20 with 19 g water. Viscosity: 1.04 mPa·s. (The following were used for all viscosity measurements: viscosimeter from CBC Materials Co., Ltd., Tokyo, Japan. Model: Viscomate VM-10A-L, measuring sensor: PR-10-L.) Particle size distribution (PCS measurement): peak 1: d(H)=1350 nm−70.2 vol-%; peak 2: d(H)=5510 nm−27.0 vol-%; peak 3: d(H)=141 nm−2.8 vol-%.

### Homogenization:

The emulsion solution was passed through a high-pressure homogenizer (APV Products, DK-2620 Alberstlund, Model No. APV-2000) five times at 600 bar, resulting in 200 g of a low-viscosity whitish-yellow emulsion. Determination of homogeneity: 1 g homogenized emulsion solution was diluted 1:20 with 19 g water. Viscosity: 0.99 mPa·s. Particle size distribution (PCS measurement): peak 1: d(H)=568 nm−92.0 vol-%; peak 2: d(H)=4870 nm−8.0 vol-%; peak 3: No signal was present.

### Emulsion E-019

### Preparation:

50.0 g evening primrose oil was placed in a 250-mL Erlenmeyer flask. 4.0 g Lipoid S 40 (Lipoid GmbH) was then added, with stirring. The mixture was stirred on a magnetic stirrer, and after the lecithin was uniformly swelled, 13.0 g propylene glycol and 133.0 g water were slowly added dropwise, with intensive stirring, and stirring was continued for 2 h (good miscibility). 200 g of a low-viscosity whitish-yellow emulsion was obtained. Determination of homogeneity: 1 g emulsion solution was diluted 1:20 with 19 g water. Viscosity: 1.03 mPa·s. Particle size distribution (PCS measurement): peak 1: d(H)=110 nm−13.3 vol-%; peak 2: d(H)=207 nm−15.1 vol-%; peak 3: d(H)=875 nm−71.6 vol-%.

### Homogenization:

This emulsion solution was passed through the high-pressure homogenizer five times at 600 bar, resulting in 200 g of a low-viscosity whitish-yellow emulsion. 1 g homogenized emulsion solution was diluted 1:20 with 19 g water. Viscosity: 0.99 mPa·s. Particle size distribution (PCS measurement): peak 1: d(H)=335 nm−100 vol-%; peak 2: d(H): No signal was present.

### Surface Charge:

A surface charge of 6.65 μmol negative charge/g emulsion was determined by CAS. However, a positive surface charge would be necessary. The reason is that in the production of the lecithin (isolation from soybeans or eggs), the choline group is sometimes split off, so that the negatively charged phosphate group remains. This results in a reduction of the surface charge of the emulsion particles, and thus may possibly result in a neutral or even weakly negative surface charge of the emulsified particles.

### Emulsion E-020

Preparation: 50.0 g evening primrose oil was placed in a 250-mL Erlenmeyer flask. 6.7 g Phosal 50 PG (Phospholipid GmbH) was then added, with stirring. 10.3 g propylene glycol and 133.0 g water were slowly added dropwise to the mixture, with intensive stirring on the magnetic stirrer, and stirring was continued for 2 h (contains oil droplets). Determination of homogeneity: 1 g emulsion solution was diluted 1:20 with 19 g water. Viscosity: 0.98 mPa·s. Particle size distribution (PCS measurement): peak 1: d(H)=1150 nm−82.6 vol-%; peak 2: d(H)=215 nm−12.8 vol-%; peak 3: d(H)=5500 nm−4.6 vol-%.

### Homogenization:

The emulsion solution was passed through the high-pressure homogenizer five times at 600 bar, resulting in 200 g of a low-viscosity whitish-yellow emulsion containing oil droplets. Determination of homogeneity: 1 g homogenized emulsion solution was diluted 1:20 with 19 g water. Viscosity: 0.99 mPa·s. Particle size distribution (PCS measurement): peak 1: d(H)=679 nm−100 vol-%; peak 2: d(H): No signal was present.

### Surface Charge:

A positive electrical potential was achievable by CAS (titration with 0.01 N HCl). Thus, in principle it was demonstrated that the emulsified droplets have a positive surface charge. However, the long-term stability of the emulsion was unsatisfactory.

### Emulsion E-021

### Preparation:

25.0 g evening primrose oil was placed in a 250-mL Erlenmeyer flask. 2.0 g Varisoft BTMS flakes were then added, with stirring. The mixture was heated to 75° C. in an oil bath, with continued stirring for 2 h, resulting in a viscous white mixture. 66.5 g water was placed in a second 250-mL Erlenmeyer flask, and 6.5 g propylene glycol was added. The mixture was heated to 60° C., and then added dropwise to the hot oil phase (27 g), with intensive stirring. The emulsion solution was allowed to cool to room temperature, with stirring, resulting in 100 g of a highly viscous whitish-yellow emulsion, pH=4.2. Determination of homogeneity: 1 g emulsion solution was diluted 1:100 with 99 g water. Viscosity: 1.14 mPa·s. Particle size distribution (PCS measurement): peak 1: d(H)=618 nm−100 vol-%; peak 2: d(H): No signal was present.

### Surface Charge:

The CAS measurements (titration with 0.001 N poly(vinylsulfonic acid sodium salt)) resulted in a surface charge of the emulsified droplets of 1.03 μmol positive charge/g emulsion.

Use of behenyl trimethylammonium methosulfate instead of lecithin as cationic emulsifier demonstrated that a highly viscous yet stable emulsion may be produced. The particle size distribution showed a monodisperse emulsion droplet size having a hydrodynamic diameter of 618 nm.

### Emulsion E-022

### Preparation:

25.0 g evening primrose oil was placed in a 250-mL Erlenmeyer flask. 2.0 g Varisoft BTMS flakes were then added, with stirring. The mixture was heated to 75° C. in an oil bath, with continued stirring for 2 h. 1.5 g lecithin was then added, with continued stirring for 10 min, resulting in a viscous whitish-yellow mixture. 65.0 g water was placed in a second 250-mL Erlenmeyer flask, and 6.5 g propylene glycol was added. The mixture was heated to 60° C., and then added dropwise to the hot oil phase (28.5 g), with intensive stirring. The emulsion solution was allowed to cool to room temperature, with stirring, resulting in 100 g of a highly viscous whitish-yellow emulsion, pH=5.2. Determination of homogeneity: 1 g emulsion solution was diluted 1:100 with 99 g water. Viscosity: 1.18 mPa·s. Particle size distribution (PCS measurement): peak 1: d(H)=755 nm−100 vol-%; peak 2: d(H): No signal was present.

### Surface Charge:

The CAS measurements (titration with 0.001 N poly(vinylsulfonic acid sodium salt)) resulted in a surface charge of the emulsified droplets of 0.29 μmol positive charge/g emulsion.

As expected, the addition of lecithin reduced the surface charge of the emulsion droplets compared to E-021. The particle size distribution, the same as for E-021, showed a monodisperse emulsion droplet size having a hydrodynamic diameter of 755 nm. This means that the lecithin as well as the behenyl trimethylammonium methosulfate were incorporated into the same emulsion droplets.

### Emulsion E-023

The lecithin emulsion was combined with a calcium chloride solution to test whether phosphate groups of two lecithin molecules together with CaCl₂ form sparingly soluble calcium phosphate. The chloride ions that are released are subsequently available to the trialkylammonium groups of the lecithin as counterions. Due to the associated shielding of the strongly anionic phosphate groups, the lecithin emulsion should acquire a cationic surface charge. For a lecithin molar mass of M=approximately 760 g/mol, 4.0 g lecithin corresponds to 5.3 mmol. That is, one-half as much calcium chloride needs to be added in order to shield all of the phosphate groups of the lecithin used. An excess of salt, which would destabilize the emulsion, is likewise avoided.

### Preparation:

50.0 g evening primrose oil was placed in a 250-mL Erlenmeyer flask. 4.0 g lecithin was then added, with stirring. The mixture was stirred on a magnetic stirrer, and after the lecithin was uniformly swelled, 13.0 g propylene glycol and 133.0 g calcium chloride solution (0.3 g CaCl₂ in 132.7 g water) were slowly added dropwise, with intensive stirring, and stirring was continued for 4 h. A low-viscosity, whitish-yellow two-phase mixture formed. Upon standing, a whitish-yellow suspension phase on the top and a clear, aqueous phase on the bottom immediately formed.

### Homogenization:

The emulsion solution was passed through the high-pressure homogenizer (APV Products, DK-2620 Alberstlund, Model No. APV-2000) 15 times at 600 bar, resulting in a low-viscosity whitish-yellow, unstable emulsion. Summary: The addition of electrolyte for E-018 and E-023 caused an unstable emulsion in the region of the charge zero point, which ruptured.

### Emulsion E-026

The addition of electrolyte for E-018 and E-023 caused an unstable emulsion in the region of the charge zero point, which ruptured. To avoid this, the lecithin was reloaded in the nonaqueous system in order to satisfactory produce the emulsion using the emulsifier, which was then cationic. In the process, the charge zero point of the emulsion solution must not be passed through, since the emulsion is not produced until afterward, using the lecithin which is already cationic. For this purpose, the lecithin was reloaded in the nonaqueous system, using an aluminum chloride cosolvent solution and a co-emulsifier suitable for stabilizing the emulsion, in order to produce the emulsion using the cationic lecithin.

For a lecithin molar mass of M=approximately 760 g/mol, 4.0 g lecithin corresponds to 5.3 mmol. That is, one-third as much aluminum chloride needs to be added in order to shield all of the phosphate groups of the lecithin used. An excess of salt, which would destabilize the emulsion, is likewise avoided.

### Preparation of Lecithin in the Oil Phase:

400 g evening primrose oil was placed in a 250-mL Erlenmeyer flask. 32 g lecithin was then added, with stirring. The mixture was stirred on a magnetic stirrer until the lecithin was uniformly swelled, resulting in 432 g of a yellow lecithin in the oil phase.

Preparation of E-026—A with 0 Equivalents Aluminum Chloride in Ethylene Glycol (Blank Value):

54 g lecithin in the oil phase was placed in a 1-L Erlenmeyer flask, and 16.7 g cosolvent/co-emulsifier solution (12.7 g ethylene glycol and 4 g Marlipal 013/30) was slowly added dropwise, with intensive stirring. Marlipal® 013/30 is an alcohol ethoxylate, available from Sasol, containing a C₁₃ chain and 3 ethylene oxide groups.

In turn, 129 g water (including 400 μL 37% formaldehyde solution as a preservative) was slowly added dropwise, with intensive stirring, with continued stirring overnight. 200 g of a whitish-yellow mixture was obtained (no phase separation). This emulsion solution was passed through the high-pressure homogenizer five times at 600 bar, resulting in 150 g of a low-viscosity white emulsion; pH=6.3; Conductivity=455 μS/cm. 1 g homogenized emulsion solution was diluted 1:40 with 39 g water, resulting in a viscosity of 1.06 mPa·S. Particle size distribution: peak 1: d(H)=153 nm−100 vol-% (PCS measurement); peak 2: d(H): No signal was present.

Preparation of E-026-B with 0.5 Equivalents Aluminum Chloride in Ethylene Glycol:

54 g lecithin in the oil phase was placed in a 250-mL Erlenmeyer flask, and 16.8 g aluminum chloride cosolvent/co-emulsifier solution (0.12 g aluminum chloride in 12.7 g ethylene glycol and 4 g Marlipal 013/30) was slowly added dropwise, with intensive stirring. In turn, 129 g water (including 400 μL 37% formaldehyde solution as a preservative) was slowly added dropwise, with intensive stirring, and stirring was continued for 4 h. 200 g of a whitish-yellow mixture was obtained (no phase separation). This emulsion solution was passed through the high-pressure homogenizer five times at 600 bar, resulting in 150 g of a low-viscosity white emulsion; pH=3.0 (aluminum chloride reacts acidically in the aqueous phase); Conductivity=1234 μS/cm. 1 g homogenized emulsion solution was diluted 1:40 with 39 g water; Viscosity: 0.98 mPa·s; Particle size distribution: peak 1: d(H)=527 nm−100 vol-%; (PCS measurement); peak 2: d(H): No signal was present.

Preparation of E-026-C with 0.75 Equivalents Aluminum Chloride in Ethylene Glycol:

54 g lecithin in the oil phase was placed in a 250-mL Erlenmeyer flask, and 16.9 g aluminum chloride cosolvent/co-emulsifier solution (0.18 g aluminum chloride in 12.7 g ethylene glycol and 4 g Marlipal 013/30) was slowly added dropwise, with intensive stirring. In turn, 129 g water (including 400 μL 37% formaldehyde solution as a preservative) was slowly added dropwise, with intensive stirring, and stirring was continued for 4 h. 200 g of a whitish-yellow mixture was obtained (phase separation overnight). This emulsion solution was passed through the high-pressure homogenizer five times at 600 bar, resulting in 150 g of a low-viscosity white emulsion; pH=2.6 (aluminum chloride reacts acidically in the aqueous phase); Conductivity=1807 μS/cm. 1 g homogenized emulsion solution was diluted 1:40 with 39 g water. Viscosity: 0.98 mPa·s; Particle size distribution: peak 1: d(H)=792 nm−100 vol-% (PCS measurement); peak 2: d(H): No signal was present.

Preparation of E-026-D with 1.0 Equivalents Aluminum Chloride in Ethylene Glycol:

54 g lecithin in the oil phase was placed in a 250-mL Erlenmeyer flask, and 16.9 g aluminum chloride cosolvent/co-emulsifier solution (0.23 g aluminum chloride in 12.7 g ethylene glycol and 4 g Marlipal 013/30) was slowly added dropwise, with intensive stirring. In turn, 129 g water (including 400 μL 37% formaldehyde solution as a preservative) was slowly added dropwise, with intensive stirring, and stirring was continued for 4 h. 200 g of a whitish-yellow mixture was obtained (no phase separation). This emulsion solution was passed through the high-pressure homogenizer five times at 600 bar, resulting in 150 g of a low-viscosity white emulsion; pH=2.6 (aluminum chloride reacts acidically in the aqueous phase); Conductivity=2160 μS/cm. 1 g homogenized emulsion solution was diluted 1:40 with 39 g water; Viscosity: 0.99 mPa·s; Particle size distribution: peak 1: d(H)=899 nm−93.8 vol-% (PCS measurement); peak 2: d(H)=5126 nm−6.2 vol-%; peak 3: d(H): No signal was present.

Preparation of E-026-E with 1.25 Equivalents Aluminum Chloride in Ethylene Glycol:

54 g lecithin in the oil phase was placed in a 250-mL Erlenmeyer flask, and 17.0 g aluminum chloride cosolvent/co-emulsifier solution (0.29 g aluminum chloride in 12.7 g ethylene glycol and 4 g Marlipal 013/30) was slowly added dropwise, with intensive stirring. In turn, 129 g water (including 400 μL 37% formaldehyde solution as a preservative) was slowly added dropwise, with intensive stirring, and stirring was continued for 4 h. 200 g of a whitish-yellow mixture was obtained (no phase separation). This emulsion solution was passed through the high-pressure homogenizer five times at 600 bar, resulting in 150 g of a low-viscosity white emulsion; pH=2.6 (aluminum chloride reacts acidically in the aqueous phase); Conductivity=2420 μS/cm. 1 g homogenized emulsion solution was diluted 1:40 with 39 g water; Viscosity: 0.99 mPa·s; Particle size distribution: peak 1: d(H)=1055 nm−100 vol-% (PCS measurement); peak 2: d(H): No signal was present.

Preparation of E-026-F with 1.5 Equivalents Aluminum Chloride in Ethylene Glycol:

54 g lecithin in the oil phase was placed in a 250-mL Erlenmeyer flask, and 17.1 g aluminum chloride cosolvent/co-emulsifier solution (0.35 g aluminum chloride in 12.7 g ethylene glycol and 4 g Marlipal 013/30) was slowly added dropwise, with intensive stirring. In turn, 129 g water (including 400 μL 37% formaldehyde solution as a preservative) was slowly added dropwise, with intensive stirring, and stirring was continued overnight. 200 g of a whitish-yellow mixture was obtained (no phase separation). This emulsion solution was passed through the high-pressure homogenizer five times at 600 bar, resulting in 150 g of a low-viscosity white emulsion; pH=2.7 (aluminum chloride reacts acidically in the aqueous phase); Conductivity=2860 μS/cm. 1 g homogenized emulsion solution was diluted 1:40 with 39 g water; Viscosity: 0.97 mPa·s; Particle size distribution: peak 1: d(H)=835 nm−97.0 vol-% (PCS measurement); peak 2: d(H)=5351 nm−3.0 vol-%; peak 3: d(H): No signal was present.

Preparation of E-026-G with 2.0 Equivalents Aluminum Chloride in Ethylene Glycol:

54 g lecithin in the oil phase was placed in a 250-mL Erlenmeyer flask, and 17.2 g aluminum chloride cosolvent/co-emulsifier solution (0.47 g aluminum chloride in 12.7 g ethylene glycol and 4 g Marlipal 013/30) was slowly added dropwise, with intensive stirring. In turn, 129 g water (including 400 μL 37% formaldehyde solution as a preservative) was slowly added dropwise, with intensive stirring, and stirring was continued for 4 h. 200 g of a whitish-yellow mixture was obtained (no phase separation). This emulsion solution was passed through the high-pressure homogenizer five times at 600 bar, resulting in 150 g of a low-viscosity white emulsion; pH=2.8 (aluminum chloride reacts acidically in the aqueous phase); Conductivity=3330 μS/cm. 1 g homogenized emulsion solution was diluted 1:40 with 39 g water; Viscosity: 0.97 mPa·s; Particle size distribution: peak 1: d(H)=0.854 nm−100 vol-% (PCS measurement); peak 2: d(H): No signal was present.

pH titration using the charge analyzing system (CAS): 0.1N HCl titration solution was used for the CAS pH titration. For preparation, i.e., cleaning, of the CAS PTFE measuring cell, before each measurement the measuring cell was rinsed in succession with deionized water, 2-propanol, deionized water, and acetone (spray bottles), and then blown with pressurized air. The PTFE measuring cell was also occasionally cleaned with 65% nitric acid. 1.0 g emulsion solution was weighed into the CAS PTFE measuring cell and filled with 9.0 g water (1:10 dilution). The measuring flask was then inserted into the measuring cell, and the pH titration on the CAS was started.

TABLE
Surface charge

| | | Isoelectric | pH | Conductivity |
|---|---|---|---|---|
| | Starting | point | (1:10 | (1:10 |
| Charge titration | potential *) | (pH for CAS) | dilution) | dilution) |
| E-026-A (0 | −321 mV | — | 5.5 | 76 μS/cm |
| equivalent) | | | | |
| E-026-B (0.5 | −122 mV | 4.0 | 3.8 | 227 μS/cm |
| equivalent) | | | | |
| E-026-C (0.75 | −29 mV | 4.8 | 3.6 | 345 μS/cm |
| equivalent) | | | | |
| E-026-D (1.0 | +38 mV | 5.0 | 3.5 | 412 μS/cm |
| equivalent) | | | | |
| E-026-E (1.25 | +55 mV | 6.8 | 3.5 | 462 μS/cm |
| equivalent) | | | | |
| E-026-F (1.5 | +70 mV | 6.4 | 3.6 | 545 μS/cm |
| equivalent) | | | | |
| E-026-G (2.0 | +99 mV | 6.5 | 3.5 | 590 μS/cm |
| equivalents) | | | | |

*) The measured starting potentials varied slightly, depending on the measurement.

### Summary for E-026-Emulsions:

Due to the Marlipal 013/30 co-emulsifier, it was possible to prepare very good, stable emulsions using different equivalents of aluminum chloride. The measuring results demonstrate very well that, as expected, the emulsions with less than 1.0 equivalent AlCl₃ have a negative surface charge. The lecithin emulsions with 1.0 or more equivalents of AlCl₃ have a cationic surface potential. It was thus possible to successfully produce a series of reloaded lecithin emulsions. In addition, CAS pH titrations were carried out for all of these emulsions. The measured starting potentials were consistent with the previously measured starting potentials. Furthermore, these measurements show very well how the pH values of the isoelectric points of the emulsions migrate toward the neutral direction with increasing aluminum chloride content. For 1.25 equivalents of AlCl₃ (E-026-E), the maximum pH occurs at pH=6.8, and for higher AlCl₃ equivalents, decreases slightly to pH=6.5 (E-026-F and E-026-G). Thus, it is apparent that the isoelectric point cannot be further shifted into the basic pH range. In addition, the pH values and conductivities were measured for all emulsions. This did not apply for the undiluted emulsions or the emulsions diluted 1:10 with deionized water. Accordingly, the maximum conductivity of the 1:10 diluted samples was 590 μS/cm for E-026-G (2.0 equivalents AlCl₃). In addition, CAS pH titrations were also carried out with different dilutions of samples. Thus, E-026-G with 2.0 equivalents AlCl₃ consistently remained cationic when undiluted, diluted 1:10, and diluted 1:50.

Emulsion E-027—Cationic Emulsion with LAE/Maltodextrin

Based on formulation E-021, but using 66% N-alpha-lauroyl-L-arginate ethyl ester monohydrochloride (LAE) with 34% maltodextrin as cationic emulsifier. The corresponding solution is available under the trade name Mirenat-D from Vedeq SA. At the same time, LAE stabilizes the emulsion against microbial degradation.

### Preparation:

50.0 g evening primrose oil was placed in a 250-mL Erlenmeyer flask. 6.0 g Mirenat-D (corresponding to 2.0 wt.-% cationic emulsifier), insoluble in oil, was then added with stirring. The mixture was stirred on a magnetic stirrer, and 144.0 g water was slowly added dropwise, with intensive stirring, and stirring was continued for 2 h (good miscibility). 200 g of a low-viscosity whitish-yellow emulsion was obtained; pH=3.4. Homogenization: This emulsion solution was passed through the high-pressure homogenizer five times at 600 bar, resulting in 150 g of a low-viscosity whitish-yellow emulsion. 1 g homogenized emulsion solution was diluted 1:20 with 19 g water; Viscosity: 0.98 mPa·s; Particle size distribution: peak 1: d(H)=228 nm−100 vol-% (PCS measurement); peak 2: d(H): No signal was present.

### Summary:

The use of N-α-lauroyl-L-arginate ethyl ester monohydrochloride (LAE) in maltodextrin instead of behenyl trimethylammonium methosulfate as cationic emulsifier showed that it was possible to produce a low-viscosity, whitish-yellow stable emulsion. The particle size distribution showed a monodisperse emulsion droplet size having a hydrodynamic diameter of 228 nm. The CAS pH titrations showed that the flow potential of the emulsion is always greater than +343 mV over the entire pH range of 3.0 to 10.0.

Emulsion E-028—Cationic Emulsion with LAE/Maltodextrin and Lecithin

Based on formulation E-022/E-027, but with lecithin as additional emulsifier.

### Preparation:

50.0 g evening primrose oil was placed in a 250-mL Erlenmeyer flask. 4.0 g lecithin (Carl Roth GmbH+Co. KG) was then added, with stirring. The mixture was stirred on a magnetic stirrer until the lecithin was uniformly swelled. 6.0 g Mirenat-D, insoluble in oil, was then added. 140.0 g water was slowly added dropwise, with intensive stirring, and stirring was continued for 2 h (good miscibility). 200 g of a low-viscosity whitish-yellow emulsion was obtained; pH=3.9. Homogenization: This emulsion solution was passed through the high-pressure homogenizer five times at 600 bar, resulting in 150 g of a low-viscosity whitish-yellow emulsion. 1 g homogenized emulsion solution was diluted 1:20 with 19 g water; Viscosity: 1.00 mPa·s; Particle size distribution: peak 1: d(H)=95 nm−19.0 vol-% (PCS measurement); peak 2: d(H)=346 nm−81.0 vol-%; peak 3: d(H): No signal was present.

### Summary:

The addition of lecithin as additional emulsifier proceeded without problems. A low-viscosity, whitish-yellow stable emulsion was obtained. The particle size distribution showed emulsion droplets having hydrodynamic diameters of 95 nm (19.0 vol-%) and 346 nm (81.0 vol-%). The CAS pH titrations showed that the flow potential of the emulsion is always greater than +229 mV over the entire pH range of 3.0 to 10.0.

Emulsion E-029—Cationic Emulsion with LAE/Glycerin

Based on formulation E-027, but with N-α-lauroyl-L-arginate ethyl ester monohydrochloride (LAE) in glycerin as cationic emulsifier. This solution, available as Aminat-G from Vedeq SA, contains 20% N-α-lauroyl-L-arginate ethyl ester monohydrochloride (LAE) in 80% glycerin.

### Preparation:

50.0 g evening primrose oil was placed in a 250-mL Erlenmeyer flask. 20.0 g Aminat-G (corresponding to 2.0 wt.-% cationic emulsifier), which did not mix well with the oil, was then mixed in with stirring. The mixture was stirred on a magnetic stirrer, and 130.0 g water was slowly added dropwise, with intensive stirring, and stirring was continued for 2 h (good miscibility). 200 g of a low-viscosity whitish-yellow emulsion was obtained; pH=4.0. Homogenization: This emulsion solution was passed through the high-pressure homogenizer five times at 600 bar, resulting in 150 g of a low-viscosity whitish-yellow emulsion. 1 g homogenized emulsion solution was diluted 1:20 with 19 g water; Viscosity: 0.99 mPa·s; Particle size distribution: peak 1: d(H)=248 nm−100 vol-% (PCS measurement); peak 2: d(H): No signal was present. The emulsion thickened after several days and formed clumps. However, the emulsion could be reliquefied by shaking.

### Summary:

The use of LAE in glycerin instead of LAE in maltodextrin as cationic emulsifier showed that it was likewise possible to produce a low-viscosity whitish-yellow emulsion. However, the emulsion thickened after several days and formed clumps, but could be reliquefied by shaking. The particle size distribution showed a monodisperse emulsion droplet size having a hydrodynamic diameter of 248 nm. The CAS pH titrations showed that the flow potential of the emulsion is always greater than +386 mV over the entire pH range of 3.0 to 10.0.

Emulsion E-030 —Cationic Emulsion with LAE/Glycerin and Lecithin

Based on formulation E-028/029, but with lecithin as additional emulsifier.

### Preparation:

50.0 g evening primrose oil was placed in a 250-mL Erlenmeyer flask. 4.0 g lecithin (Carl Roth GmbH+Co. KG) was then added, with stirring. The mixture was stirred on a magnetic stirrer until the lecithin was uniformly swelled. 20.0 g Aminat-G, which did not mix well with the oil, was then mixed in. 140.0 g water was slowly added dropwise, with intensive stirring, and stirring was continued for 2 h (good miscibility). 200 g of a low-viscosity whitish-yellow emulsion was obtained; pH=4.3. Homogenization: This emulsion solution was passed through the high-pressure homogenizer five times at 600 bar, resulting in 150 g of a low-viscosity whitish-yellow emulsion. 1 g homogenized emulsion solution was diluted 1:20 with 19 g water; Viscosity: 0.95 mPa·s; Particle size distribution: peak 1: d(H)=233 nm−100 vol-% (PCS measurement); peak 2: d(H): No signal was present.

### Summary:

The addition of lecithin as additional emulsifier proceeded without problems. A low-viscosity, whitish-yellow stable emulsion was obtained. The particle size distribution showed a monodisperse emulsion droplet size having a hydrodynamic diameter of 233 nm. The CAS pH titrations showed that the flow potential of the emulsion is always greater than +306 mV over the entire pH range of 3.0 to 10.0.

Emulsion E-031—Kilo Batch of Cationic Emulsion with LAE/Glycerin, Lecithin, and β-Carotene as Pseudo-Active Substance

Based on formulation E-030, but with β-carotene as pseudo-active substance and DL-alpha-tocopherol acetate (antioxidant) for oxidative stabilization of the evening primrose oil. The concentration of LAE as cationic emulsifier was reduced to 0.4%, since this is the maximum allowable concentration of LAE in cosmetic products (does not apply to products used as a spray). Likewise, the lecithin content was reduced to 1.6%.

### Preparation:

150.0 g evening primrose oil was placed in a 1-L Erlenmeyer flask. 9.6 g lecithin (Carl Roth GmbH+Co. KG), 3 mg DL-alpha-tocopherol acetate (97.0%, HPLC, Fluka), and 322 mg β-carotene (97.0%, UV, Fluka) were then added, with stirring. The mixture was stirred overnight, in the sealed Erlenmeyer flask flushed with nitrogen, on a magnetic stirrer until the entire mixture was dissolved. 428.1 g water was placed in a second 1-L Erlenmeyer flask, and 12.0 g Aminat-G (corresponding to 0.4% cationic emulsifier and 1.6% glycerin) was added. The mixture was then added dropwise, with intensive stirring, into the 160 g of the oil phase, and stirring was continued for 2 h. 600 g of a low-viscosity carrot red emulsion was obtained; pH=5.2. Homogenization: This emulsion solution was passed through the high-pressure homogenizer five times at 600 bar, resulting in 500 g of a low-viscosity orange-whitish emulsion. 1 g homogenized emulsion solution was diluted 1:20 with 19 g water; Viscosity: 0.98 mPa·s; Particle size distribution: peak 1: d(H)=82 nm−4.9 vol-% (PCS measurement); peak 2: d(H)=631 nm−95.1 vol-%; peak 3: d(H): No signal was present. An isoelectric point of pH=6.6 was identified in the CAS pH titration using 0.01 N sodium hydroxide

### Summary:

The production of the emulsion proceeded successfully; it was possible to produce a low-viscosity orange-whitish emulsion using β-carotene as pseudo-active substance. The particle size distribution showed emulsion droplets having hydrodynamic diameters of 82 nm (4.9 vol-%) and 631 nm (95.1 vol-%). The CAS pH titrations showed that in the acidic pH range, i.e., below pH=6.0, the flow potential of the emulsion was greater than +200 mV. However, the emulsion had an isoelectric point at pH=6.6, which indicates that 0.4% N-α-lauroyl-L-arginate ethyl ester monohydrochloride (LAE) as cationic emulsifier is not sufficient if the lecithin emulsion is to be cationic over the entire pH range.

Emulsion E-032—Kilo Batch of Cationic Emulsion with LAE/Glycerin, Lecithin, and Carotene as Pseudo-Active Substance

Based on formulation E-031, but with 0.8% instead of 0.4% LAE as cationic emulsifier. 0.8% is the maximum allowable concentration of LAE in soaps and antidandruff shampoos (does not apply to products used as a spray).

### Preparation:

Same as for E-031, but with provision of 416.1 g water and 24.0 g Aminat-G, resulting in 600 g of a low-viscosity carrot red emulsion; pH=4.6. Homogenization: Same as for E-031.500 g of a low-viscosity orange-whitish emulsion was obtained. 1 g homogenized emulsion solution was diluted 1:20 with 19 g water; Viscosity: 0.96 mPa·s; Particle size distribution: peak 1: d(H)=126 nm−8.3 vol-% (PCS measurement); peak 2: d(H)=827 nm−80.6 vol-%; peak 3: d(H)=5208 nm−11.0 vol-%.

A charge titration in a 1:10 dilution using the charge analyzing system (CAS) with anionic polyelectrolyte solution (0.001 N poly(vinylsulfonic acid sodium salt) resulted in a measured surface charge of 12.05 μmol positive charge/g emulsion (+96.5 mC/g emulsion).

### Summary:

The production of the emulsion using 0.8% LAE proceeded successfully. It was possible to produce a low-viscosity orange-whitish emulsion using β-carotene as pseudo-active substance. The particle size distribution showed emulsion droplets having hydrodynamic diameters of 126 nm (8.3 vol-%), 827 nm (80.6 vol-%), and 5208 nm (11.0 vol-%). According to the charge titration, the surface charge of the cationic emulsion was 12.05 μmol positive charge/g emulsion. The CAS pH titrations showed that the flow potential of the emulsion was always greater than +238 mV over the entire pH range of 3.0 to 10.0. Consequently, the emulsion has no isoelectric point in this pH range. 0.8% N-α-lauroyl-L-arginate ethyl ester monohydrochloride (LAE) as cationic emulsifier was therefore sufficient to make the lecithin emulsion cationic over the stated pH range. As a result, the loading of textiles may also take place in slightly alkaline tap water containing lime without having to slightly acidify the water in each case with citric acid, for example.

Emulsion E-033—1.2 kg Batch of Cationic, Active Substance-Free Emulsion with LAE/Glycerin and Lecithin

The preparation was analogous to E-032, but without the use of β-carotene as pseudo-active substance. This cationic emulsion was used to load the finished textiles for the absorption and desorption tests.

The production of the active substance-free emulsion proceeded successfully; it was possible to produce a low-viscosity whitish-yellow emulsion. The particle size distribution showed a monodisperse emulsion droplet size having a hydrodynamic diameter of 303 nm. According to charge titration, the surface charge of the cationic emulsion was 12.12 μmol positive charge/g emulsion. The pH titrations showed that the flow potential of the emulsion was always greater than +198 mV over the entire pH range of 3.0 to 10.0.

### E. Application of the Active Substance Emulsion to the Finishing Layer; Absorption

### Test 1

0.2 g of emulsion E-021 was diluted in 6 mL water, and the entire amount was sprayed onto 4.4 g of finished fabric sample A-007 (containing mPEG 1000 methacrylate). After it was completely dry, the sample was extracted and methylated. The GC analysis showed 423.6 μg palmitic acid methyl ester/g sample, which corresponds to 6 mg evening primrose oil/g sample.

### Test 2 (S-004)

Absorption test: 39.2 g water was placed in a 100-mL Schott flask, and 0.8 g cationic BTMS emulsion E-021 was added. A 4.0 g fabric sample A-009 was then placed in the flask and shaken, then allowed to stand for 16 h at room temperature. The fabric sample was then dried for 1 h in a drying oven at 50° C. Leaching was visually observable.

Quantification: 50 mg of fabric sample S-004 was weighed three times each into 2-mL sample vials, using tweezers. In each case, 1000 μL INT-STD-01 (standard toluene solution containing 1 mg/mL nonadecanoic acid methyl ester) for extraction and 100 μL 5% methanolic meta-catalyst solution were pipetted in, using a micropipette. The 2-mL sample vials were sealed, extracted for 5 min on a mechanical shaker, and allowed to stand at room temperature for at least 30 min. To remove the catalyst (protection of the GC column), 50±5 mg Amberlyst 15 was added in each case to the mixtures and shaken briefly. The mixtures were then centrifuged, the supernatant was transferred into GC vials, and the content determinations were carried out on the GC/MS. The GC analysis showed 267 μg/cm² evening primrose oil, which corresponded to 35 mg evening primrose oil/g sample.

### Test 3

0.4 g of E-021 emulsion was diluted in 40 mL water. 4.0 g of finished fabric sample A-009 (containing HEMA) was immersed and shaken. The active substance emulsion leached out from the fabric immediately.

### Test 4 (S-005, S-006)

Absorption tests for E-033 (LAE emulsion) on A-084 (finishing with P-044, containing 40% mPEG 350 methacrylate and 20% 2-ethylhexyl acrylate), and on A-088 (finishing with P-067, containing 20% mPEG 350 methacrylate and 40% 2-ethylhexyl acrylate) over a period of 30 min for determination of loading capacity (absorption capability). In dermatological practice, the quantities of skin care products applied to the skin are typically 2 mg/cm², with an active substance content of 1-3% and a contact time of normally 16 hours. This was taken into account in the subsequent absorption tests.

Fabric samples: A-084: Anionic PA fabric containing poly(2-acrylamido-2-methylpropane sodium sulfonate-stat-2-ethylhexyl acrylate-stat-N-(butoxymethyl)acrylamide) with P-044 after one machine wash at 60° C. for 50-55 min. A-088: Anionic PA fabric containing poly(2-acrylamido-2-methylpropane sodium sulfonate-stat-2-ethylhexyl acrylate-stat-N-(butoxymethyl)acrylamide) with P-067 after one machine wash at 60° C. for 50-55 min. PA fabric: 100% PA fabric (tricot), Fussenegger Textilveredelung GmbH, prefixed charmeuse, weight=135 g/m², width=140 cm, color=0109.

For the sample preparation, the finished PA fabric sample's, after undergoing a machine wash at 60° C. for 50-55 min, were cut to a size (approximately 20×20 cm) having a weight of 5.0 g in each case. In each case 71.0 g water was placed in 150-mL metal pressure cylinders, and 4.0 g cationic LAE emulsion E-033 was added. 5.0 g of fabric sample A-084 (S-005) or 5.0 g of fabric sample A-088 (S-006) was placed in each of the metal pressure cylinders, which were immediately inserted into a Polymat operated at 50 rpm at 25° C. (cycle: 12 s rotation, 3 pause, 12 s counterrotation, etc.). After 30 min, each of the fabric samples together with the liquor was centrifuged separately at 2800 rpm for exactly 1 min at room temperature, in a spin dryer which had been previously cleaned with spray cleaner, sponge, washcloth, and water, and was then air-dried at room temperature. The blank value was prepared in the same manner.

Gravimetric analysis resulted in the following loading values using evening primrose oil (% of the theoretical maximum value of 1 g): S-005, 30 min: 90%; S-006, 30 min: 91%; blank value: 8%.

The specific disclosed embodiments should not be construed as limiting the scope of the present invention. Based on the preceding description and the drawings, a person skilled in the art is aware of various possible variations and modifications, besides the disclosed examples, which are likewise intended to fall under the scope of protection of the claims.

1. An emulsion for loading a textile product with at least one low-molecular compound, the at least one low-molecular compound being contained in a dispersed phase of the emulsion, wherein a surface of particles of the dispersed phase has a positive charge.

2. The emulsion according to claim 1, wherein the emulsion contains lecithin and/or quaternary ammonium compounds containing one or two long-chain lipophilic radicals as a surface-active compound.

3. The emulsion according to claim 1, wherein at least 90 vol-% of the particles of the dispersed phase of the emulsion have a hydrodynamic diameter of less than 1000 nm.

4. The emulsion according to claim 1, wherein the emulsion is an oil-in-water emulsion, and the at least one low-molecular compound is present in the lipophilic dispersed phase.

5. The emulsion according to claim 1, wherein the emulsion is a water-in-oil-in-water emulsion, and the at least one low-molecular compound is present in the aqueous dispersed phase within the lipophilic dispersed phase.

6. The emulsion according to claim 1, further comprising liposomes, wherein the at least one low-molecular compound is present in the aqueous phase within the liposomes.

7. The emulsion according to claim 1, wherein the surface charge of the particles of the dispersed phase of the emulsion is at least 15 mC/g emulsion.

8-9. (canceled)

10. A method for loading a textile product with a low-molecular compound, comprising:
a) providing a textile product with a finishing layer whose accessible surface has a negative charge; and
b) contacting the textile product with an emulsion, the at least one low-molecular compound being contained in a dispersed phase of the emulsion, and a surface of particles of the dispersed phase having a positive charge.

11. The method according to claim 10, wherein step b) is repeated.

12. (canceled)

13. A polymer compound, comprising an acrylic acid copolymer composed of acrylic acid derivatives and/or methacrylic acid derivatives, containing: a) at least one acrylic acid derivative and/or methacrylic acid derivative substituted with a sulfonic acid group; b) at least one hydrophilically substituted acrylic acid derivative and/or methacrylic acid derivative; c) at least one lipophilically substituted acrylic acid derivative and/or methacrylic acid derivative; and d) at least one acrylic acid derivative and/or methacrylic acid derivative which acts as a crosslinking agent.

14. The polymer compound according to claim 13, wherein the acrylic acid copolymer contains 2-acryloyl-2-methylpropanesulfonic acid.

15. The polymer compound according to claim 13, wherein the acrylic acid copolymer contains ethyl triglycol methacrylate, 2-hydroxyethyl methacrylate, and/or mPEG methacrylate as a hydrophilic monomer.

16. The polymer compound according to claim 13, wherein the acrylic acid copolymer contains 2-ethylhexyl acrylate as a lipophilic monomer.

17. The polymer compound according to claim 13, wherein the at least one crosslinking monomer of the acrylic acid copolymer is selected from the group consisting of N-(butoxymethyl)acrylamide, N-(methylol)acrylamide, glycidyl methacrylate, p-EMKO-TDI-o-HEMA, and EMKO-2-(N-(tert-butyl){[(3-isocyanato-1,5,5-trimethylcyclohexyl)methyl]amino}carbonylamino)ethyl methacrylate.

18. The polymer compound according to claim 13, wherein the polymer compound contains polyethersulfones, polyurethanes, polyester urethanes, polyether urethanes, polyamides, or mixtures thereof.

19. A finishing formulation for finishing a textile product or for coating a wound dressing, comprising a polymer compound according to claim 13.

20. A finishing layer on a textile product or a wound dressing, comprising a polymer compound according to claim 13.

21. A textile product, comprising a finish, the finish comprising a polymer compound according to claim 13.

22. A wound dressing comprising a finish containing a polymer compound according to claim 13.

23. A method for loading a textile product with a low-molecular compound, comprising:
a) providing a textile product with a finishing layer whose accessible surface has a negative charge; and
b) contacting the textile product with a solution, the at least one low-molecular compound being cationic and being dissolved in the solution.
